(19) United States
(12) Patent Application Publication
(10) Pub. No.: US 2022/0127620 A1
(43) Pub. Date: Apr. 28, 2022

(54) MICROBIAL PRODUCTION OF COMPOUNDS

(71) Applicants: Andrew P. KLEIN, Emeryville, CA (US); Amyris, Inc., Emeryville, CA (US)

(72) Inventors: Christopher D. REEVES, Emeryville, CA (US); Christopher J. PADDON, Emeryville, CA (US); Victor F. HOLMES, Emeryville, CA (US); Andrew P. KLEIN, Emeryville, CA (US)

(21) Appl. No.: 17/438,810

(22) PCT Filed: Mar. 13, 2020
(86) PCT No.: PCT/US2020/022741

§ 371 Date: Sep. 13, 2021

Related U.S. Application Data
(60) Provisional application No. 62/819,457, filed on Mar. 15, 2019.

(51) Int. Cl. C12N 15/52; C12N 1/18; C12N 9/10; C12N 9/88; C12P 7/42

(52) U.S. Cl. CPC C12N 15/52; C12N 1/18; C12N 9/1029; C12Y 203/01206; C12N 2500/32; C12Y 404/01026; C12P 7/42; C12N 2500/34; C12N 9/88

(57) ABSTRACT

Provide are modified host cells that are engineered to decrese expression of a product to undetectable levels in the presence of an exogenous agent, and increase expression of the product in the presence of another exogenous agent. The modified yeast strainshost cells do not express detectable levels of a precursor or substrate used to make the product. The product can be a cannabinoid or precursor thereof, and the substrate can be hexanoate. Also provided are methods for making a product using the modified host cells. The modified host cell can be a yeast strain, such as S. cerevisiae.

### CROSS REFERENCES TO RELATED APPLICATIONS

The present application is a US National Phase Application Under Section 371 of PCT/US2020/022741 filed Mar. 13, 2020, which claims priority to U.S. Provisional Pat. Appl. No. 62/819,457, filed on Mar. 15, 2019, which applications are incorporated herein by reference intheir entireties.

### SEQUENCE LISTING

The instant application contains a Sequence Listing which has been submitted electronically in ASCII format and is hereby incorporated by reference in its entirety. Said ASCII copy, created on Sep. 1, 2021, is named 101928-1263196-001010US_SL.txt and is 94,711 bytes in size.

### BACKGROUND OF THE INVENTION

When organisms are used to produce biomolecules, it is usually beneficial to have an externally-controlled genetic switch to toggle between a high-growth, low-production mode (appropriate for biomass generation and ease of handling) and a low-growth, high-production mode (appropriate for profitable manufacture). For most fermentatively-produced biomolecules, a single switch mechanism is sufficient, even though it allows as much as 10-20% production in the low-production mode. For some biomolecules, such as cannabinoids, regulatory requirements create a need for extremely low or non-detectable production in the low-production state.

There are many examples of strong single-mechanism switches found in nature, including the galactose regulation system of yeast and the arabinose regulation system in bacteria. Most are based on normal physiological responses where genes are activated when the organism senses a threat or resource. There are also several systems that respond to molecules not usually found in an organisms' environment—tetracycline, IPTG, indigo—that have been used in biotechnological applications.

### BRIEF SUMMARY OF THE INVENTION

In one aspect, a modified, engineered or recombinant host cell is provided, the host cell comprising a heterologous genetic pathway that produces a heterologous product and that is regulated by an exogenous agent, wherein the host cell does not produce a precursor required to make the product. In some embodiments, the exogenous agent comprises a regulator of gene expression.

In some embodiments, the exogenous agent decreases production of the heterologous product. In some embodiments, the exogenous agent that decreases production of the heterologous product is glucose and expression of one or more enzymes encoded by the heterologous genetic pathway are under control of a glucose repressed promoter.

In some embodiments, the exogenous agent increases production of the heterologous product. In some embodiments, the exogenous agent that increases production of the heterologous product is galactose and expression of one or more enzymes encoded by the heterologous genetic pathway are under control of a GAL promoter.

In some embodiments, the heterologous genetic pathway comprises a galactose-responsive promoter, a maltose-responsive promoter, or a combination of both.

In some embodiments, the heterologous product is a cannabinoid or cannabinoid precursor. In some embodiments, the cannabinoid or cannabinoid precursor is cannabidiolic acid (CBDA), cannabidiol (CBD), cannabigerolic acid (CBGA), or cannabigerol (CBG).

In some embodiments, the genetic pathway encodes at least two enzymes selected from the group consisting of hexanoyl-CoA synthase (HCS), tetraketide synthase (TKS) and olivetolic acid cyclase (OAC).

In some embodiments, the precursor required to make the product is hexanoate.

In some embodiments, the heterologous genetic pathway comprises a nucleic acid construct comprising at least 3 protein coding regions.

In some embodiments, the host cell is a yeast cell or yeast strain. In some embodiments, the yeast cell is S. cerevisiae.

In another aspect, a mixture is provided, the mixture comprising a host cell described herein and a culture media. In some embodiments, the culture media comprises an exogenous agent that decreases production of the heterologous product. In some embodiments, the exogenous agent that decreases production of the heterologous product is glucose, maltose, or lysine.

In some embodiments, the culture media comprises (i) an exogenous agent that increases production of the heterologous product, and (ii) a precursor required to make the heterologous product. In some embodiments, the exogenous agent that increases production of the heterologous product is galactose. In some embodiments, the precursor required to make the heterologous product is hexanoate.

In another aspect, a method for decreasing the expression of a heterologous product is provided, the method comprising culturing a host cell described herein in a media comprising the exogenous agent, wherein the exogenous agent decreases the expression of the heterologous product. In some embodiments, the exogenous agent that decreases expression of the heterologous product is glucose, maltose, or lysine. In some embodiments, culturing the host cell strain in the media comprising the exogenous agent results in less than 0.001 mg/L of heterologous product

In another aspect, a method for increasing the expression of a heterologous product is described, the method comprising culturing a host cell described herein in a media comprising the exogenous agent, wherein the exogenous agent increases expression of the heterologous product. In some embodiments, the exogenous agent that increases expression of the heterologous product is galactose.

In some embodiments, the method further comprises culturing the host cell with the precursor required to make the heterologous product. In some embodiments, the precursor required to make the heterologous product is hexanoate.

In some of the embodiments described herein, the heterologous product is a cannabinoid or cannabinoid precursor. In some embodiments, the cannabinoid or cannabinoid precursor is CBDA, CBD, CBGA, or CBG.

In another aspect, a host cell is provided, the host cell comprising a heterologous genetic pathway that produces a cannabinoid and is regulated by an exogenous agent. In some embodiments, the host cell does not comprise a precursor required to make the cannabinoid, or does not comprise an amount of precursor required to make the cannabinoid above a predetermined level (e.g., greater than 10 mg/L). In some embodiments, the host cell does not comprise hexanoate at a level sufficient to make the cannabinoid in an amount over 10 mg/L. In some embodiments, the cannabinoid is CBDA, CBD, CBGA, or CBG.

In some embodiments, the exogenous agent downregulates expression of the heterologous genetic pathway. In some embodiments, the exogenous agent that downregulates expression of the heterologous genetic pathway is glucose. In some embodiments, the expression of one or more enzymes encoded by the heterologous genetic pathway are under control of a glucose repressed promoter.

In some embodiments, the exogenous agent upregulates expression of the heterologous genetic pathway. In some embodiments, the exogenous agent that upregulates expression of the heterologous genetic pathway is galactose. In some embodiments, the expression of one or more enzymes encoded by the heterologous genetic pathway are under control of a GAL promoter.

In some embodiments, the genetic pathway encodes at least two enzymes selected from the group consisting of hexanoyl-CoA synthase (HCS), tetraketide synthase (TKS) and olivetolic acid cyclase (OAC).

In some of the aspects or embodiments described herein, the host cell can be a yeast cell or yeast strain. In some of the aspects or embodiments described herein, the yeast cell is S. cerevisiae.

In another aspect, a method for decreasing expression of a cannabinoid is provided, the method comprising culturing a host cell described herein in a media comprising an exogenous agent, wherein the exogenous agent decreases the expression of the cannabinoid or a precursor thereof. In some embodiments, the exogenous agent that decreases the expression of the cannabinoid or a precursor thereof is glucose, maltose, or lysine. In some embodiments, culturing the host cell in the media comprising the exogenous agent results in less than 0.001 mg/L of cannabinoid or a precursor thereof

In another aspect, a method for increasing expression of a cannabinoid is provided, the method comprising culturing a host cell described herein in a media comprising the exogenous agent, wherein the exogenous agent increases the expression of the cannabinoid or a precursor thereof. In some embodiments, the exogenous agent that increases the expression of the cannabinoid or a precursor thereof that is galactose. In some embodiments, the method further comprises culturing the host cell in a media comprising hexanoate.

In some of the aspects or embodiments described herein, the cannabinoid or cannabinoid precursor is CBDA, CBD, CBGA, or CBG.

### BRIEF DESCRIPTION OF THE DRAWINGS

FIG. 1 shows expression of the cannabinoid precursors olivetol and olivetolic acid by the modified host cells described herein, as described in Example 1.

FIG. 2 and FIG. 3 show genetic maps of the heterologous nucleic acids transformed into the modified host cells as described in Example 1.

FIG. 4 shows part the cannabinoid synthetic pathway referred to herein.

FIG. 5 is a schematic showing the structure and function of a maltose regulated transcriptional switch as used herein.

FIG. 6 shows the biochemical pathways for the synthesis of cannabigerol (CBG) and cannabidiol (CBD) from sugar derived geranyl pyrophosphate (GPP) and from hexanoic acid.

FIG. 7 shows the general layout of CBDA synthase (CBDAS) surface-display constructs arranged from the N to the C terminus.

FIG. 8, FIG. 9, and FIG. 10 are pairs of graphs showing normalized biomass (upper graph each) and normalized CBDA titers (lower graph each) under conditions of no hexanoic acid or 2 mM hexanoic acid where each is also measured under conditions of 4% maltose, 2% maltose and 2% sucrose, and 4% sucrose for the strains Y61508 (FIG. 8); Y66316 (FIGS. 9); and Y66085 (FIG. 10).

### DEFINITIONS

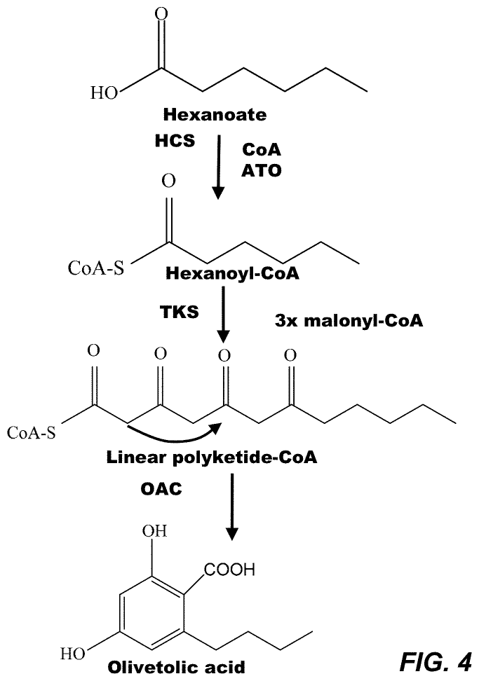

A “genetic pathway” as used herein refers to a set of at least two different coding sequences, where the coding sequences encode enzymes that catalyze different parts of a synthetic pathway to form a desired product. In a genetic pathway a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway. An example of a cannabinoid synthetic pathway is shown in FIG. 4.

As used herein, the term “endogenous” refers to a substance or process that can occur naturally in a host cell. In contrast, the term “exogenous” refers a substance or compound that originated outside an organism or cell. The exogenous substance or compound can retain its normal function or activity when introduced into an organism or host cell described herein.

The terms “modified,” “recombinant” and “engineered,” when used to modify a host cell described herein, refer to host cells or organisms that do not exist in nature, or express compounds, nucleic acids or proteins at levels that are not expressed by naturally occurring cells or organisms.

As used herein, the term “genetically modified” denotes a host cell that comprises a heterologous nucleotide sequence. The genetically modified host cells described herein typically do not exist in nature.

The term “heterologous compound” refers to the production of a compound by a cell that does not normally produce the compound, or to the production of a compound at a level at which it is not normally produced by the cell.

As used herein, the term “heterologous” refers to what is not normally found in nature. The term “heterologous compound” refers to the production of a compound by a cell that does not normally produce the compound, or to the production of a compound at a level not normally produced by the cell. For example a cannabinoid can be a heterologous compound.

As used herein, the phrase “heterologous enzyme” refers to an enzyme that is not normally found in a given cell in nature. The term encompasses an enzyme that is: (a) exogenous to a given cell (i.e., encoded by a nucleotide sequence that is not naturally present in the host cell or not naturally present in a given context in the host cell); and (b) naturally found in the host cell (e.g., the enzyme is encoded by a nucleotide sequence that is endogenous to the cell) but that is produced in an unnatural amount (e.g., greater or lesser than that naturally found) in the host cell.

A “heterologous genetic pathway” as used herein refers to a genetic pathway that does not normally or naturally exist in an organism or cell.

As used herein, the phrase “operably linked” refers to a functional linkage between nucleic acid sequences such that the linked promoter and/or regulatory region functionally controls expression of the coding sequence.

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “productivity” refers to production of a compound by a host cell, expressed as the amount of non-catabolic compound produced (by weight) per amount of fermentation broth in which the host cell is cultured (by volume) over time (per hour).

As used herein, the term “promoter” refers to a synthetic or naturally-derived nucleic acid that is capable of activating, increasing or enhancing expression of a DNA coding sequence, or inactivating, decreasing, or inhibiting expression of a DNA coding sequence. A promoter may comprise one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible or repressible.

The term “yield” refers to production of a compound by a host cell, expressed as the amount of compound produced per amount of carbon source consumed by the host cell, by weight.

The term “about” when modifying a numerical value or range herein includes normal variation encountered in the field, and includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

### DETAILED DESCRIPTION OF THE INVENTION

Provided herein are recombinant or modified host cells that are useful for producing a heterologous product, and methods of using the host cells. The recombinant or modified host cells comprise a heterologous genetic pathway that can be differentially regulated by one or more exogenous agents. The recombinant host cells provide the advantage of decreasing expression of the heterologous product to below exceedingly low, and preferably undetectable levels under one set of conditions, while allowing robust expression of the heterologous product under a second set of conditions. In some embodiments, the host cell is engineered to express heterologous enzymes in the cannabinoid pathway. In some embodiments, the host cell is a yeast cell.

### Modified Host Cells Comprising a Heterologous Genetic Pathway

In one aspect, provided herein are host cells comprising a heterologous genetic pathway that produces a heterologous product. In some embodiments, the heterologous genetic pathway comprises a genetic regulatory element, such as a nucleic acid sequence, that is regulated by an exogenous agent. In some embodiments, the exogenous agent acts to regulate expression of the heterologous genetic pathway. Thus, in some embodiments, the exogenous agent can be a regulator of gene expression.

In some embodiments, the exogenous agent can be used as a carbon source by the host cell. For example, the same exogenous agent can both regulate expression of the heterologous genetic pathway and provide a carbon source for growth of the host cell. In some embodiments, the exogenous agent is glucose. In some embodiments, the exogenous agent is galactose. In some embodiments, the exogenous agent is maltose.

In some embodiments, the genetic regulatory element is a nucleic acid sequence, such as a promoter. In some embodiments, the genetic regulatory element is a glucose-responsive promoter or a promoter that is repressed by glucose. In some embodiments, glucose negatively regulates expression of the heterologous genetic pathway, thereby decreasing production of the heterologous product. Exemplary glucose repressed promoters include pMAL11, pMAL12, pMAL13, pMAL21, pMAL22, pMAL31, pMAL32, pMAL33, pCAT8, pHXT2, pHXT4, pMTH1, and pSUC2.

TABLE 1
Exemplary Glucose Repressed Promoter Sequences

| Promoter | Sequence |
|---|---|
| pMAL11 | SEQ ID NO: 21 |
| pMAL12 | SEQ ID NO: 22 |
| pMAL13 | SEQ ID NO: 25 |
| pMAL21 | SEQ ID NO: |
| pMAL22 | SEQ ID NO: 26 |
| pMAL31 | SEQ ID NO: 23 |
| pMAL32 | SEQ ID NO: 24 |
| pMAL33 | SEQ ID NO: 27 |
| pCAT8 | SEQ ID NO: 28 |
| pHXT2 | SEQ ID NO: 29 |
| pHXT4 | SEQ ID NO: 30 |
| pMTH1 | SEQ ID NO: 31 |
| pSUC2 | SEQ ID NO: 32 |

In some embodiments, the genetic regulatory element is a galactose-responsive promoter. In some embodiments, galactose positively regulates expression of the heterologous genetic pathway, thereby increasing production of the heterologous product. In some embodiments, the galactose-responsive promoter is a GAL1 promoter. In some embodiments, the galactose-responsive promoter is a GAL10 promoter. In some embodiments, the galactose-responsive promoter is a GAL2, GAL3, or GALT promoter. In some embodiments, heterologous genetic pathway comprises the galactose-responsive regulatory elements described in Westfall et al. (PNAS (2012) vol. 109: E111-118). In some embodiments, the host cell lacks the gal1 gene and is unable to metabolize galactose, but galactose can still induce galactose-regulated genes.

TABLE 2
Exemplary GAL Promoter Sequences

| Promoter | Sequence |
|---|---|
| pGAL1 | SEQ ID NO: 13 |
| pGAL10 | SEQ ID NO: 14 |
| pGAL2 | SEQ ID NO: 15 |
| pGAL3 | SEQ ID NO: 16 |
| pGAL7 | SEQ ID NO: 17 |
| pGAL4 | SEQ ID NO: 18 |

In some embodiments, the galactose regulation system used to control expression of heterologous genes is re-configured such that it is no longer induced by the presence of galactose. Instead, the genes will be expressed unless repressors, which may be lysine in some strains or maltose in other strains, are present in the media.

In some embodiments, the genetic regulatory element is a maltose-responsive promoter. In some embodiments, maltose negatively regulates expression of the heterologous genetic pathway, thereby increasing production of the heterologous product. In some embodiments, the maltose maltose-responsive promoter is selected from the group consisting of pMAL1, pMAL2, pMAL11, pMAL12, pMAL31 and pMAL32. The maltose genetic regulatory element can be designed to both activate expression of some genes and repress expression of others, depending on whether maltose is present or absent in the medium. Maltose regulation of gene expression and maltose-responsive promoters are described in U.S. Patent Publication 2016/0177341, which is hereby incorporated by reference. Genetic regulation of maltose metabolism is described in Novak et al., “Maltose Transport and Metabolism in S. cerevisiae,” Food Technol. Biotechnol. 42 (3) 213-218 (2004).

TABLE 3
Exemplary MAL Promoter Sequences

| Promoter | Sequence |
|---|---|
| pMAL1 | SEQ ID NO: 19 |
| pMAL2 | SEQ ID NO: 20 |
| pMAL11 | SEQ ID NO: 21 |
| pMAL12 | SEQ ID NO: 22 |
| pMAL31 | SEQ ID NO: 23 |
| pMAL32 | SEQ ID NO: 24 |

In some embodiments, the heterologous genetic pathway is regulated by a combination of the maltose and galactose regulons.

In some embodiments, the heterologous genetic pathway is regulated by lysine. The regulation of LYS genes is described, for example, by Feller et al., Eur. J. Biochem. 261, 163-170 (1999).

In some embodiments, the recombinant host cell does not comprise, or expresses a very low level of (for example, an undetectable amount), a precursor required to make the heterologous product. In some embodiments, the precursor is a substrate of an enzyme in the heterologous genetic pathway.

### Cannabinoid Pathway

In another aspect, the host cell comprises a heterologous genetic pathway that produces a cannabinoid or a precursor of a cannabinoid. In some embodiments, the precursor is a substrate in the cannabinoid pathway. In some embodiments, the precursor is a substrate for hexanoyl-CoA synthase (HCS), tetraketide synthase (TKS), or olivetolic acid cyclase (OAC). In some embodiments, the precursor, substrate or intermediate in the cannabinoid pathway is hexanoate, olivetol, or olivetolic acid. In some embodiments, the precursor is hexanoate. In some embodiments, the host cell does not comprise the precursor, substrate or intermediate in an amount sufficient to produce the cannabinoid or a precursor of the cannabinoid. In some embodiments, the host cell does not comprise hexanoate at a level or in an amount sufficient to produce the cannabinoid in an amount over 10 mg/L. In some embodiments, the heterologous genetic pathway encodes at least two enzymes selected from the group consisting of hexanoyl-CoA synthase (HCS), tetraketide synthase (TKS) and olivetolic acid cyclase (OAC). The cannabinoid pathway is described in Keasling et al. (WO 2018/200888).

In some embodiments, the host cell is a yeast strain. In some embodiments, the yeast strain is a Y27600, Y27602, Y27603, or Y27604 strain.

### Yeast Strains

In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the strain is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorphs (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the strain is Saccharomyces cerevisiae. In some embodiments, the host is a strain of Saccharomyces cerevisiae selected from the group consisting of Baker's yeast, CEN.PK, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is selected from the group consisting of PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

In some embodiments, the strain is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

In some embodiments, the yeast strain is a Y27598, Y27599, Y27600, Y27601 Y27602, Y27603, Y27604 or Y25618 strain. Exemplary yeast strains are shown in Table 4 below.

TABLE 4
Yeast Strains

| | | Mega- | Stitch | Stitch | | | Genes | | | |
|---|---|---|---|---|---|---|---|---|---|---|
| Strain | Parent | stitches | A | B | locus | SNAP | expressed | HCS | TDS | OAC |
| Y27599 | Y27036 | 101227 | 89523 | 78270 | GAS4 | CAIB | 2xTKS | 0 | 2 | 0 |
| Y27601 | Y27039 | 101226 | 85240 | 89531 | GAS2 | HC | 2x TKS | 0 | 4 | 0 |
| | | 101227 | 89523 | 78270 | GAS4 | CAIB | 2xTKS | | | |
| Y27598 | Y27036 | 96695 | 85217 | 78270 | GAS4 | CAIB | HCS, | 1 | 1 | 0 |
| | | | | | | | TKS | | | |
| Y27600 | Y21791 | 101225 | 85240 | 85234 | GAS2 | HC | HCS, | 1 | 3 | 0 |
| | | | | | | | TKS | | | |
| | | 101227 | 89523 | 78270 | GAS4 | CAIB | 2x TKS | | | |
| Y27602 | Y27039 | 101226 | 85240 | 89531 | GAS2 | HC | 2x TKS | 1 | 3 | 0 |
| | | 96695 | 85217 | 78270 | GAS4 | CAIB | HCS, | | | |
| | | | | | | | TKS | | | |
| Y25618 | Y27039 | 96692 | 85221 | 85231 | GAS2 | HC | HCS, 2x | 1 | 2 | 1 |
| | | | | | | | TKS, | | | |
| | | | | | | | OAC | | | |
| Y27603 | Y27039 | 101225 | 85240 | 85234 | GAS2 | HC | TKS, | 2 | 2 | 2 |
| | | | | | | | HCS | | | |
| | | 101224 | 85217 | 89528 | GAS4 | CAIB | HCS, | | | |
| | | | | | | | TKS, 2x | | | |
| | | | | | | | OAC | | | |
| Y27604 | Y27039 | 101229 | 89524 | 85234 | GAS2 | HC | 2x | 2 | 2 | 4 |
| | | | | | | | OAC, | | | |
| | | | | | | | TKS, | | | |
| | | | | | | | HCS | | | |
| | | 101224 | 85217 | 89528 | GAS4 | CAIB | HCS, | | | |
| | | | | | | | TKS, 2x | | | |
| | | | | | | | OAC | | | |

### Mixtures

In another aspect, provided are mixtures of the host cells described herein and a culture media described herein. In some embodiments, the culture media comprises an exogenous agent described herein. In some embodiments, the culture media comprises an exogenous agent that decreases production of the heterologous product. In some embodiments, exogenous agent that decreases production of the heterologous product is glucose or maltose.

In some embodiments, the culture media comprises an exogenous agent that increases production of the heterologous product. In some embodiments, the exogenous agent that increases production of the heterologous product is galactose. In some embodiments, the culture media comprises a precursor or substrate required to make the heterologous product. In some embodiments, the precursor required to make the heterologous product is hexanoate. In some embodiments, the culture media comprises an exogenous agent that increases production of the heterologous product and a precursor or substrate required to make the heterologous product. In some embodiments, the exogenous agent that increases production of the heterologous product is galactose, and the precursor or substrate required to make the heterologous product is hexanoate.

### Methods of Making the Host Cells

In another aspect, provided are methods of making the modified host cells described herein. In some embodiments, the methods comprise transforming a host cell with the heterologous nucleic acid constructs described herein encoding the proteins expressed by a heterologous genetic pathway described herein. Methods for transforming host cells are described in “Laboratory Methods in Enzymology: DNA”, Edited by Jon Lorsch, Volume 529, (2013); and U.S. Pat. No. 9,200,270 to Hsieh, Chung-Ming, et al., and references cited therein.

### Methods for Producing a Heterologous Product

In another aspect, methods are provided for producing a heterologous product described herein. In some embodiments, the method decreases expression of a heterologous product. In some embodiments, the method comprises culturing a host cell comprising a heterologous genetic pathway described herein in a media comprising an exogenous agent, wherein the exogenous agent decreases the expression of the heterologous product. In some embodiments, the exogenous agent is glucose or maltose. In some embodiments, the method results in less than 0.001 mg/L of heterologous product. In some embodiments, the heterologous product is a cannabinoid or a precursor thereof.

In some embodiments, the method is for decreasing expression of a cannabinoid product or precursor thereof. In some embodiments, the method comprises culturing a host cell comprising a heterologous cannabinoid pathway described herein in a media comprising an exogenous agent, wherein the exogenous agent decreases the expression of the cannabinoid or a precursor thereof. In some embodiments, the exogenous agent is glucose or maltose. In some embodiments, the method results in the production of less than 0.001 mg/L of cannabinoid or a precursor thereof.

In some embodiments, the method increases the expression of a heterologous product. In some embodiments, the method comprises culturing a host cell comprising a heterologous genetic pathway described herein in a media comprising the exogenous agent, wherein the exogenous agent increases expression of the heterologous product. In some embodiments, the exogenous agent is galactose. In some embodiments, the method further comprises culturing the host cell with the precursor or substrate required to make the heterologous product.

In some embodiments, the method increases the expression of a cannabinoid product or precursor thereof. In some embodiments, the method comprises culturing a host cell comprising a heterologous cannabinoid pathway described herein in a media comprising an exogenous agent, wherein the exogenous agent increases the expression of the cannabinoid or a precursor thereof. In some embodiments, the exogenous agent is galactose. In some embodiments, the method further comprises culturing the host cell with a precursor or substrate required to make the heterologous cannabinoid product or precursor thereof. In some embodiments, the precursor required to make the heterologous cannabinoid product or precursor thereof is hexanoate. In some embodiments, the combination of the exogenous agent and the precursor or substrate required to make the heterologous cannabinoid product or precursor thereof produces a higher yield of cannabinoid than the exogenous agent alone.

In some embodiments, the cannabinoid or a precursor thereof is cannabidiolic acid (CBDA), CBD, cannabigerolic acid (CBGA), or CBG.

### Nucleic Acids

Due to the inherent degeneracy of the genetic code, other polynucleotides which encode substantially the same or functionally equivalent polypeptides can also be used to clone and express the polynucleotides encoding the protein components of the heterologous genetic pathway described herein.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8).

Those of skill in the art will recognize that, due to the degenerate nature of the genetic code, a variety of DNA molecules differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25: 365-89).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used comparing a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in a host cell, for example, a yeast.

In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorphs, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous ADA genes, proteins, or enzymes, techniques may include, but are not limited to, cloning a gene by PCR using primers based on a published sequence of an ADA gene/enzyme or by degenerate PCR using degenerate primers designed to amplify a conserved region among ADA genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above mentioned databases in accordance with the teachings herein.

In some embodiments, the nucleic acid sequences encode proteins or polypeptides having at least 80%, 85%, 90%, 91%, 92%, 93%, 94%, 95%, 96%, 97%, 98%, 99% or 100% sequence identity to the amino acid sequence of a protein or enzyme encoded by a heterologous genetic pathway described herein. In some embodiments, the nucleic acid sequences encode proteins or polypeptides having at least 80%, 85%, 90%, 91%, 92%, 93%, 94%, 95%, 96%, 97%, 98%, 99% or 100% sequence identity to the amino acid sequence of HCS, TKS, or OAC.

### Culture and Fermentation Methods

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing heterologous products provided herein may be performed in a suitable culture medium (e.g., with or without pantothenate supplementation) in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag GmbH & Co. KDaA, Weinheim, Germany.

In some embodiments, the culture medium is any culture medium in which a genetically modified microorganism capable of producing a heterologous product can subsist, i.e., maintain growth and viability. In some embodiments, the culture medium is an aqueous medium comprising assimilable carbon, nitrogen and phosphate sources. Such a medium can also include appropriate salts, minerals, metals and other nutrients. In some embodiments, the carbon source and each of the essential cell nutrients, are added incrementally or continuously to the fermentation media, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable media for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose, in the culture medium should promote cell growth, but not be so high as to repress growth of the microorganism used. Typically, cultures are run with a carbon source, such as glucose, being added at levels to achieve the desired level of growth and biomass. Production of heterologous products may also occur in these culture conditions, but at undetectable levels (with detection limits being about <0.1 g/1). In other embodiments, the concentration of a carbon source, such as glucose, in the culture medium is greater than about 1 g/L, preferably greater than about 2 g/L, and more preferably greater than about 5 g/L. In addition, the concentration of a carbon source, such as glucose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, preferably greater than about 2.0 g/L and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 ml/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of compounds of interest. For example, prior to inoculation of the culture medium with an inoculum, the culture medium can be brought to and maintained at a temperature in the range of from about 20.degree. C. to about 45.degree. C., preferably to a temperature in the range of from about 25.degree. C. to about 40.degree. C., and more preferably in the range of from about 28.degree. C. to about 32.degree. C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose is used as a carbon source the glucose is preferably fed to the fermentor and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

### EXAMPLES

### Example 1

### Host Cells Engineered with the Cannabinoid Synthetic Pathway

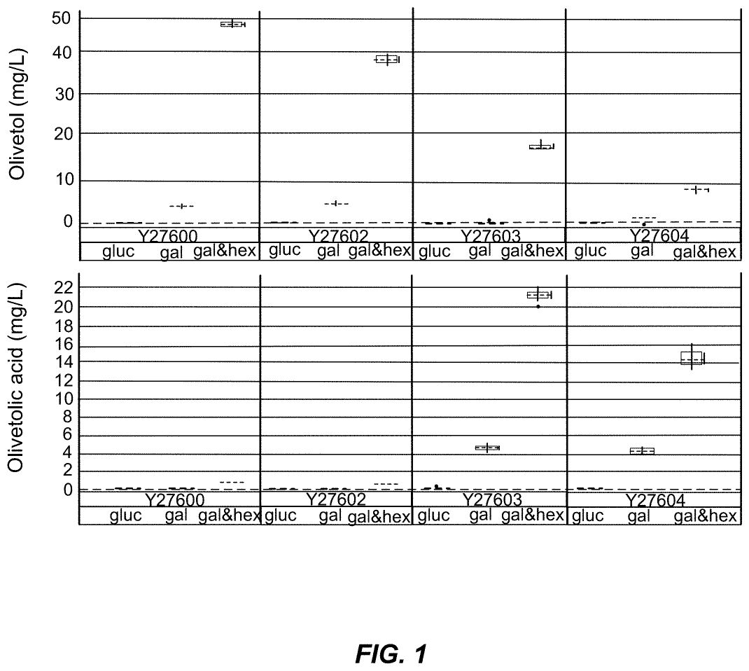

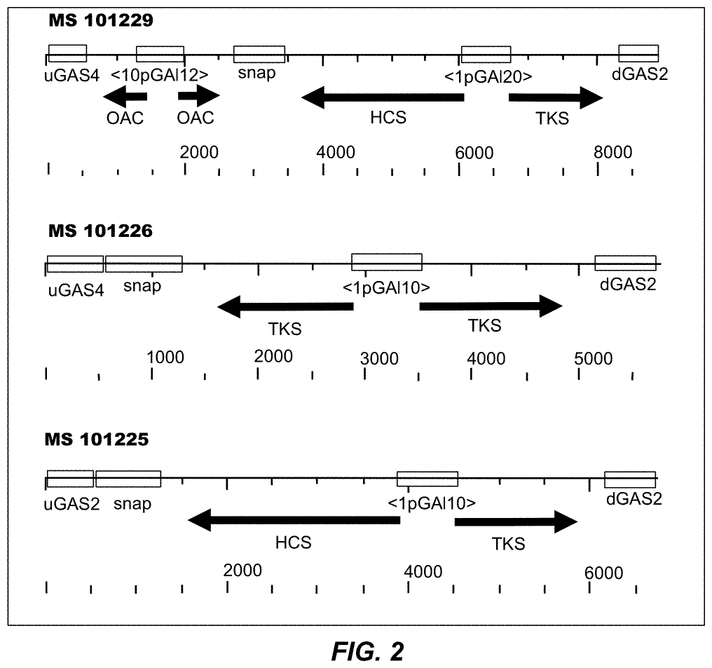

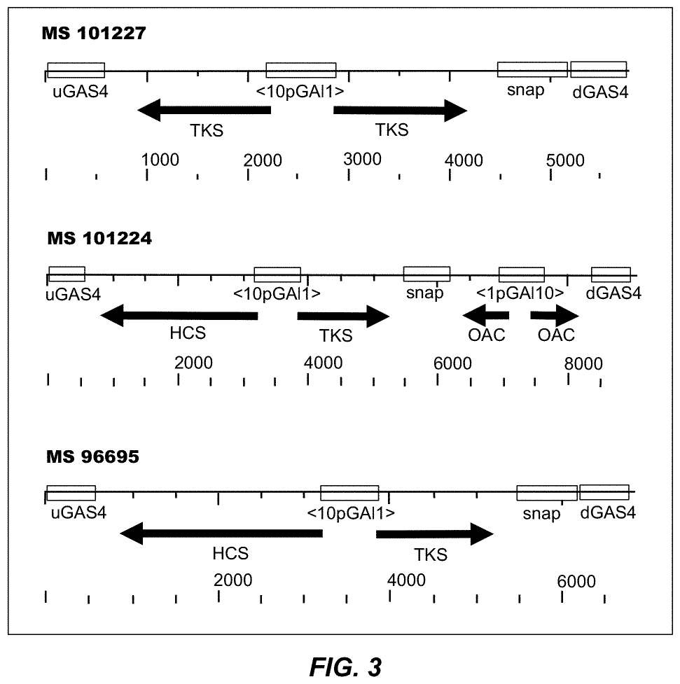

Yeast were engineered to express part the cannabinoid synthetic pathway. As shown in FIG. 4, the enzymes hexanoyl-CoA synthase (HCS), tetraketide synthase (TKS) and olivetolic acid cyclase (OAC) synthesize olivetolic acid starting from hexanoate as a substrate. HCS uses hexanoate as a substrate to form hexanoyl-CoA, which in turn is used as a substrate by TKS to for malonyl-CoA, which in turn is used as a substrate by OAC to form olivetolic acid. Coding sequences for each of HCS, TKS, and OAC, each under the control of a GAL promoter, were inserted into S. cerevisiae yeast cells. Accordingly, synthesis of each of these enzymes was induced only if the yeast was grown in the presence of galactose. As shown in Table 4, FIG. 2 and FIG. 3, several constructs (and resulting yeast strains) were made, some of which only expressed a subset of HCS, TKS, and OAC whereas other constructs and yeast strains contained at least one copy of each of HCS, TKS, and OAC under control of the GAL promoter. Table 4, FIG. 2 and FIG. 3 are useful for understanding which strains were tested for the data shown in FIG. 1.

In the case of the cannabinoid pathway, hexanoate can be fed to provide the hexanoyl-coenzyme A substrate required for production of the polyketide precursor to cannabinoids (see FIG. 4). Wild type yeast produces very low levels of hexanoate, so if it is not fed, cannabinoid production is greatly reduced. FIG. 1 shows the level of the cannabinoid precursors olivetol and olivetolic acid produced by various yeast strains engineered for switchable expression of the pathway genes (HCS, TKS, and OAC) and grown under three conditions. In the first two conditions, no hexanoate was fed to the strains and the carbon source was either glucose (gluc; turns off pathway expression) or galactose (gal; turns on pathway expression). In the third (right-most) condition, galactose was the carbon source, which activates the pathway genes and hexanoate was fed to the yeast. As can be seen, when galactose was the carbon source and hexanote was fed to the yeast, significant amounts of the cannabinoid precursors were produced. On the other hand when glucose was the carbon source, thereby turning off expression of the cannabinoid pathway, and hexanoate was not fed, cannabinoid production was below the limit of detection of the assay (<0.001 mg/L). This example demonstrates the use of two orthogonal switching systems (galactose-induced pathway expression, and hexanoate addition) to ensure the complete turn-off of production of olivetol and olivetolic acid. Similar orthogonal switching systems in which a precursor of a pathway must be supplied exogenously in combination with a genetic switch (e.g., an induced promoter or alternatively a repressed promoter) can be used to control other heterologous pathways introduced into yeast.

### Example 2

### Yeast Transformation Methods

Each DNA construct was integrated into Saccharomyces cerevisiae (CEN.PK113-7D) using standard molecular biology techniques in an optimized lithium acetate (LiAc) transformation. Briefly, cells were grown overnight in yeast extract peptone dextrose (YPD) media at 30° C. with shaking (200 rpm), diluted to an OD600 of 0.1 in 100 mL YPD, and grown to an OD₆₀₀ of 0.6-0.8. For each transformation, 5 mL of culture was harvested by centrifugation, washed in 5 mL of sterile water, spun down again, resuspended in 1 mL of 100 mM LiAc, and transferred to a microcentrifuge tube. Cells were spun down (13,000×g) for 30 seconds, the supernatant was removed, and the cells were resuspended in a transformation mix consisting of 240 μL 50% PEG, 36 μL 1 M LiAc, 10 μL boiled salmon sperm DNA, and 74 μL of donor DNA. For transformations that required expression of the endonuclease F-Cph1, the donor DNA included a plasmid carrying the F-CphI gene expressed under the yeast TDH3 promoter for expression. This will cut the F-CphI endonuclease recognition site in the landing pad to facilitate integration of the target gene of interest. Following a heat shock at 42° C. for 40 minutes, cells were recovered overnight in YPD media before plating on selective media. DNA integration was confirmed by colony PCR with primers specific to the integrations.

### Example 2

### Generation of a Base Strain with a Genetic Switching System that is Suitable for Rapid Genetic Engineering for the Production of Non-Catabolic Compounds

To generate a strain that can be rapidly engineered to make an arbitrary natural compound, several engineering steps were performed on the original yeast isolate CEN.PK113-7D. First, a meganuclease protein was integrated into the chromosome to enable nuclease-based engineering in subsequent rounds of transformation. Second, seven chromosomal loci were engineered to gain nucleotide sequences that enable high-efficiency integration of future DNA constructs using validated nucleases. Third, a maltose-responsive genetic switch was added to control the expression of genes driven by GAL promoters (pGALx). The resulting strain Y46850 serves as a chassis into which designs for natural compound biosynthesis may be rapidly prototyped.

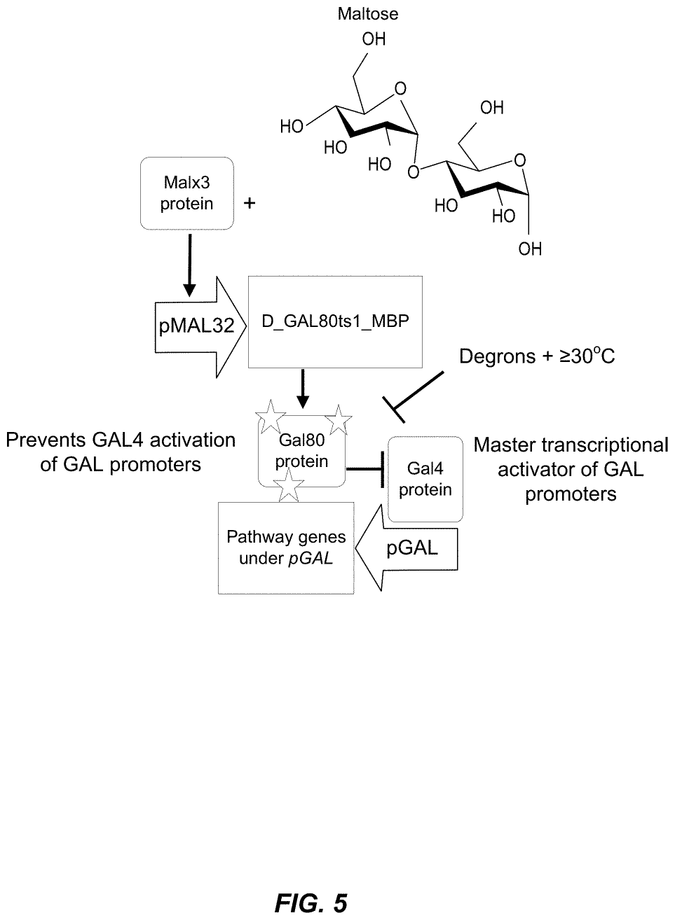

The invention and uses of the maltose-responsive genetic switch were previously described in WO2016210350; US201615738555; and US201615738918, each of which are incorporated herein by reference in their entireties. In brief, the genetic switch enables a heterologous, non-catabolic pathway to switch between On and Off states in response to maltose and temperature (FIG. 5). When the strain is grown in the presence of maltose and at temperatures <28° C., the expression of all pGALx-driven genes will be Off, allowing cellular resources to instead go toward the generation of biomass, i.e. growth. Conversely, when the strain is grown in the absence of maltose and at temperatures >30° C., the expression of all pGALx-driven genes will be On, enabling high-yield conversion of fed sucrose into a non-catabolic product.

The maltose switch is a GAL80 based switch, wherein a maltose-responsive promoter drives expression of GAL80 (pMALx>GAL80). A challenge of GAL80 based switches is that mutations that reactivate Ga180p activity in fermentations will shut down biosynthetic production, an event favored by natural selection. Two major approaches were developed to reduce GAL80 reactivation. First, a UBR1-targeted degron (D) was fused to a temperature sensitive GAL80 (GAL80ts1) to speed up Ga180 protein degradation when maltose is depleted and the temperature is >30° C. Second, the GAL80 protein was further destabilized by fusing a maltose binding protein (MBP) based degron onto the C-terminus. When maltose is present, the GAL80p-MBP mutant fusion protein is stable; however, when maltose is depleted, the GAL80 protein is quickly degraded. Another benefit of using the MBP mutant is that strains with D_GAL80ts1 MBP showed significantly lower “leakiness” of GAL gene expression during growth in OFF-state conditions.

### Example 3

### Generation of a Strain Capable of Producing Cannabigerolic Acid (CBGA)

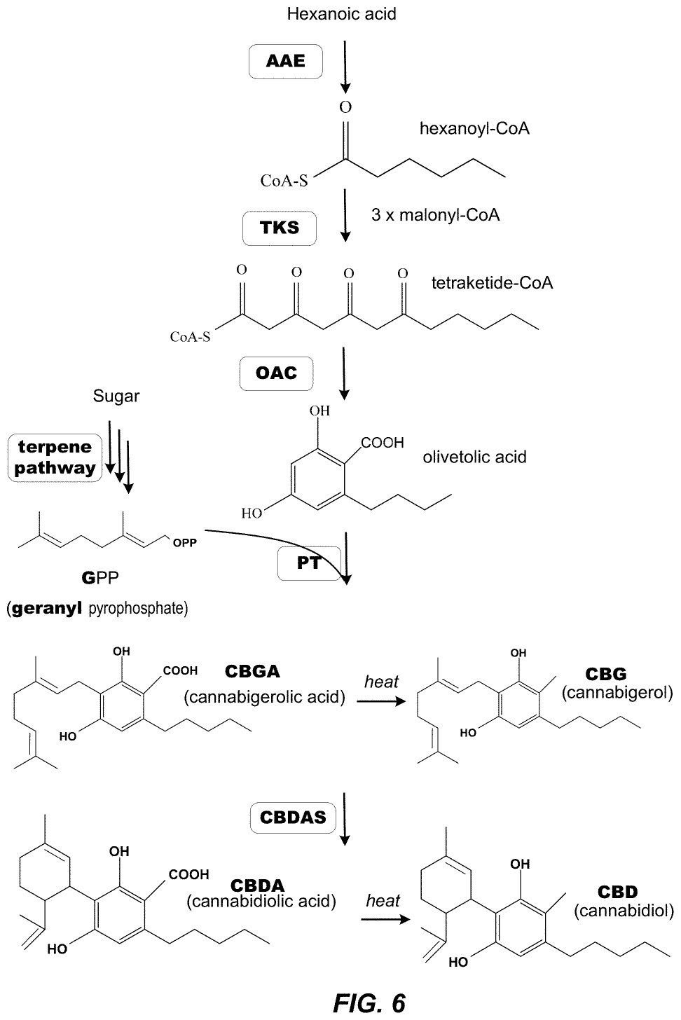

A set of genes capable of producing the cannabinoid CBGA was engineered into strain Y46850 in three steps (Table 5 and FIG. 6). First, constructs were integrated into chromosomal loci to express three heterologous genes from Cannibis sativa AAE, TKS, and OAC, together with the Zymomonas mobilis PDC gene and two endogenous S. cerevisiae ACS1 and ALD6 genes were, all using pGALx promoters. Second, constructs were integrated into chromosomal loci to express seven endogenous genes of the S. cerevisiae mevalonate pathway (ERG10, ERG13, catalytic domain of HMG1, ERG12, ERGS, MVD1, and IDI1). Third, constructs were integrated into chromosomal loci to express Streptomyces aculeolatus GPPS and Cannabis sativa CBGa synthase (CBGAS) gene. The CBGAS gene required extensive N-terminal engineering to enable its expression in a catalytically active form and that did not inhibit the growth of yeast. This engineering is described elsewhere (forthcoming patent application on DPL1-PT4 engineering and TM78-hop chimeragenesis). The resulting strain Y61508 is capable of producing CBGA when fed a mixture of sucrose and hexanoic acid, as described in the Yeast culturing conditions section below.

Notably, genes involved in the production of hexanoic acid have not been engineered into this strain. Endogenous yeast metabolism produces a negligible amount of hexanoic acid or hexanoyl-CoA, which means the strains are dependent on the exogenous supply of hexanoic acid to produce cannabinoids (FIG. 6).

TABLE 5

| Enzyme | SEQ ID NOs | Promoter |
|---|---|---|
| Sc.ACS1 | | pGAL10 |
| Sc.ALD6 | | pGAL1 |
| Zm.PDC | | pGAL7 |
| Cs.AAE | | 2 × pGAL10 |
| Cs.TKS | 11 | 2 × pGAL10 |
| Cs.OAC | 12 | 4 × pGAL1 |
| Sc.ERG10 | | pGAL2 |
| Sc.ERG13 | | pGAL1 |
| Sc.HMG1-truncated | | pGAL10 |
| Sc.ERG12 | | pGAL2 |
| Sc.ERG8 | | pGAL1 |
| Sc.MVD1 | | pGAL10 |
| Sc.IDI1 | | pGAL7 |
| Sa.GPPS | | pGAL10 |
| Sc.DPL1-Cs.PT4 fusion | | pGAL1 |
| CWP2_CBDAS_12aalink4_SAG1 | | pGAL1 |

### Example 4

### Generation of a Strain Capable of Producing Cannabidiolic Acid (CBDA)

Cannabidiolic acid synthase (CBDAS) is an oxidative cyclase that creates a carbon-carbon bond to fold the geranyl moiety of CBGA into a 6-member ring. CBDAS belongs to the Berberine-Bridge Enzyme family that employs a bicovalently bound flavin mononucleotide in the active site to utilize molecular oxygen, and each reaction cycle also produces a molecule of hydrogen peroxide (H202). CBDAS in Cannabis sativa has disulfide bonds, is glycosylated, and is natively secreted into the apoplastic space of trichomes, which is thought to have evolved to prevent auto-toxicity via H202 generation. A further challenge to functionally expressing CBDAS in yeast is its narrow pH range of ≈4.5-5.

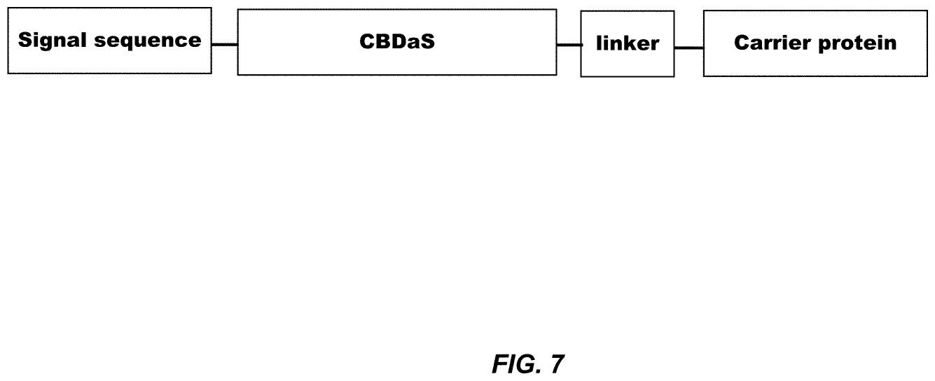

Yeast surface display is a classic molecular biology technique where a protein of interest is hosted on the exterior surface of yeast cells, allowing the protein to interact directly with the media. Surface display fulfills the requirements for CBDAS activity as surface proteins are glycosylated (emanating from the Golgi), and the pH of fermentation media is low. Surface display is preferable to secretion, as pumping protein into the broth could lead to foaming issues. To design a protein construct for CBDAS surface display, we selected the yeast cell wall mannoprotein CWP2 to supply the signal sequence and SAG1 to serve as a carrier protein (FIG. 7 and Table 5). This construct was integrated into a nearly isogenic sibling of strain Y61508 to generate strain Y66085.

### Example 5

### Yeast Culturing Conditions

For routine strain characterization in a 96-well-plate format, yeast colonies were picked into a 1.1-mL-per-well capacity 96-well ‘PreCulture plate’ filled with 360 μL per well of Pre-Culture media. Pre-Culture media consists of Bird Seed Media (BSM, originally described by van Hoek et al., (2000), Biotechnology and Bioengineering, vol. 68, pp. 517-523) at pH 5.05 with 14 g/L sucrose, 7 g/L maltose, 3.75g/L ammonium sulfate, and 1 g/L lysine. Cells were cultured at 28° C. in a high capacity microtiter plate incubator shaking at 1000 rpm and 80% humidity for 3 days until the cultures reached carbon exhaustion.

The growth-saturated cultures were sub-cultured by taking 14.4 μL from the saturated cultures and diluting into into a 2.2-mL-per-well capacity 96-well ‘Production plate’ filled with 360 μL per well of Production media. Production media consists of BSM at pH 5.05 with 40 g/L sucrose, 3.75 g/L ammonium sulfate, and 2 mM hexanoic acid. Cells in the production media were cultured at 30° C. in a high capacity microtiter plate shaker at 1000 rpm and 80% humidity for an additional 3 days prior to extraction and analysis.

### Example 6

### Analytical Methods for Cannabinoid Extraction and Titer Determination

At the conclusion of the incubation of the Production plate, methanol is added to each well such that the final concentration is 67% (v/v) methanol. An impermeable seal is added, and the plate is shaken at 1000 rpm for 30 seconds to lyse the cells and extract cannabinoids. The plate is centrifuged for 30 seconds at 200×g to pellet cell debris. 300 μL of the clarified sample is moved to an empty 1.1-mL-capacity 96-well plate and sealed with a foil seal. The sample plate is stored at −20C until analysis

Cannabidiolic acid (CBDA) and cannabigerolic acid (CBGA) were separated using a Thermo Vanquish Series UPLC-UV system with an Accupore Polar Premium 2.6 μm C18 column (100×2.1 mm). The mobile phase was a gradient of 5 mM Ammonium Formate with 0.1% formic acid aqueous solution and 0.1% formic acid in acetonitrile at a flow rate of 1.2 ml/min. Calibration curves were prepared by weight in the extraction solvent using neat standards.

### Example 7

### Validation of Two Orthogonal Switching Systems

For some biomolecules, such as cannabinoids, regulatory requirements create a need for extremely low or non-detectable production during the growth phase required to propagate the strain. To this end, the geneticly encoded maltose-responsive switch was combined with the dependency on exogenously supplied hexanoic acid for cannabinoid biosynthesis.

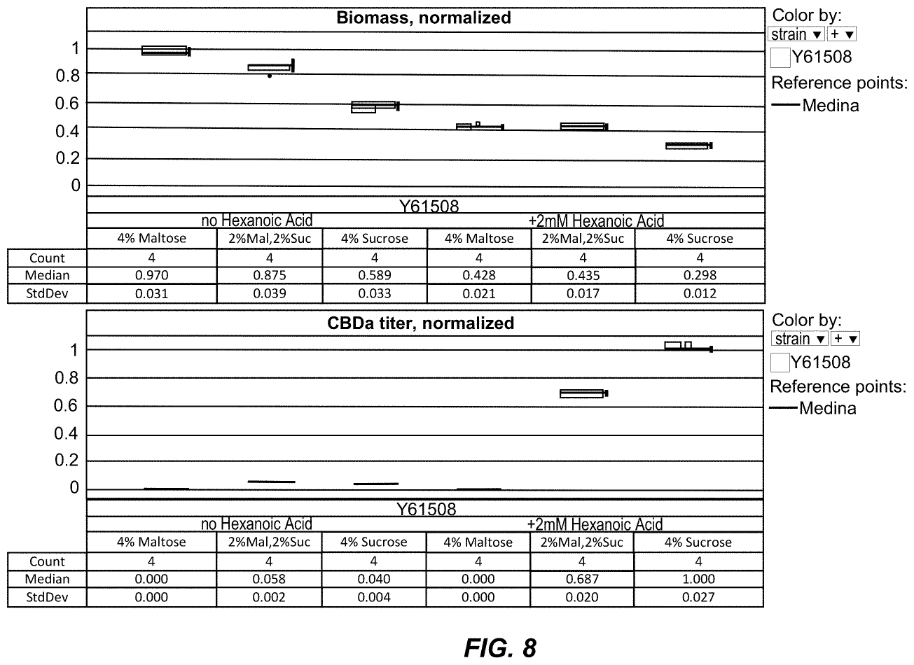

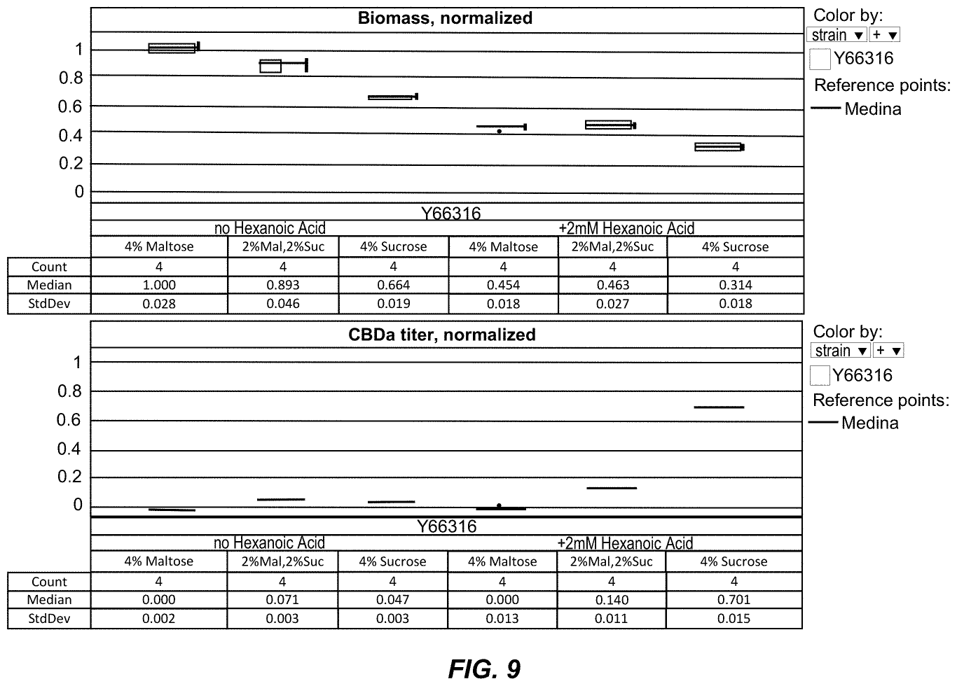

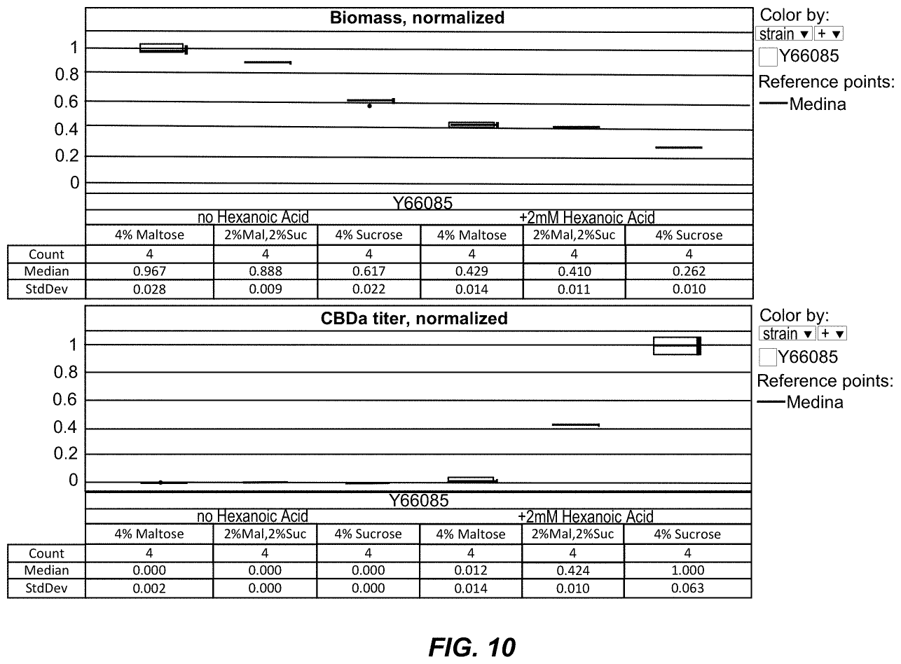

When strain Y61508 was grown in the absence of maltose and the presence of hexanoic acid, the highest CBGA titer and lowest biomass accumulation was observed (FIG. 8), which is consistent with the channeling of cellular resources into this non-catabolic pathway. As the sucrose was replaced by maltose, the CBGA titer decreases and the biomass accumulation increases. When exogenous hexanoic acid is no longer supplied, the CBGA titer also decreases and the biomass accumulation increases. Highly similar results were observed for the distinct CBGA-producing strain Y66316 (FIG. 9).

Importantly, the highest biomass accumulation and lowest CBGA titer was observed when these strains were grown in the presence of 4% maltose and without the exogenous supply of hexanoic acid. In this condition, cannabinoid production was below the limit of detection of the assay (<0.001 mg/L). This example demonstrates the use of two orthogonal switching systems to ensure the complete turn-off of cannabinoid production and channeling of cellular resources instead to biomass accumulation, i.e. growth.

To extend this finding, we tested the CBDA-production strain Y66085 in the same conditions. Once again, the absence of maltose and the exogenous supply of hexanoic acid allowed the cells to switch fully into cannabinoid production at the expense of growth (FIG. 10). By substituting the sucrose with maltose, or by removing the exogenous supply of hexanoic acid, the CBDA titer decreased and biomass increased. This example demonstrates the use of two orthogonal switching systems extends to multiple strains engineered to produce different cannabinoids.

It is understood that the examples and embodiments described herein are for illustrative purposes only and that various modifications or changes in light thereof will be suggested to persons skilled in the art and are to be included within the spirit and purview of this application and scope of the appended claims. All publications, including genbank accession numbers, patents, and patent applications cited herein are hereby incorporated by reference in their entirety for all purposes.

INFORMAL SEQUENCE LISTING
MS101225 (SEQ ID NO: 1):
GACGGCACGGCCACGCGTTTAAACCGCCTACGCCATCATTAAAG
ACCTGGTCAACTATAAAATAATACAATCAATACTTGCTTGAACG
CTTGATTTTACTGATATTCTATCCAAAAGCAAGTAGACCAGAAA
CTCTCAAGATGTTGCAAATACCGTTCGATGTTTTTGGTTTAGAT
TGTTTTAATGTTGATGCTTTTTTACTTATTTTTGGAAGCGTCTT
TTTAATTTAGTTTTATATTATAGGTATATGAATGTGTTTATGCC
AATAAGGGTTTTTTTGTACAGTTATGTGATTATAAACAGTCTTT
TGTCTAGTTTTTTTCACCAGTATCGGCCTCTATTTATAAAAAAC
GGAGCAGCTTTCGGTGTCAGTAATTCTGAAAAAATTTGTGTCAC
TCTGATTGTAAATGAATTAATTTAGCTAGATAGTTGCGAGCCCC
AACGAGAAGATTGTCAGACAAAGACAACATTCAACAACCTACAT
CCGTTACTATTCGTTAACTCGAGGTACTTGAAACTTTTCAGTTA
AGTCGCTCGTCCAACGCCGGCGGACCTGCGAGTAAGCAACTCTG
GCGCTGGCATGGCATAACCGGCGACGGCAATGCGCAAGATGGGA
TGCTATGGGCAGAGAGCCGTACTTTACTGCTTATGGCACTACAG
CAACAGATGGTTACCCCACTAAGCCTGAAGCGAATCGCCATCAA
TTCTGCGCAGTGGCGAGGAGATAAAAGCGCGGAAGTCATTCATC
AACTGGCGACGCTACTCAAAGCAGGGTTAACGCTTTCTGAAGGG
CTGGCTCTGCTGGCGGAACAGCATCCCAGTAAGCAATGGCAAGC
GTTGCTGCAATCGCTGGCGCACGATCTCGAACAGGGCATTGCTT
TTTCCAATGCCTTATTACCCTGGTCAGAGGTATTTCCGCCGCTC
TATCAGGCGATGATCCGCACGGGTGAACTGACCGGTAAGCTGGA
TGAATGCTGCTTTGAACTGGCGCGTCAGCAAAAAGCCCAGCGTC
AGTTGACCGACAAAGTGAAATCAGCGTTACGTTATCCCATCATC
ATTTTAGCGATGGCAATCATGGTGGTTGTGGCAATGCTGCATTT
TGTTCTGCCGGAGTTTGCCGCTATCTATAAGACCTTCAACACCC
CACTACCGGCACTAACGCAGGGGATCATGACGCTGGCAGACTTT
AGTGGCGAATGGAGCTGGCTGCTGGTGTTGTTCGGCTTTCTGCT
GGCGATAGCCAATAAGTTGCTGAACGGCCGGCCAAGCACGCGGG
GATCAGTAGGACAAAGGGTTCTCGTAGAGTCCCCGGAAAAAAAA
AAGGACAAAAAGTTTCAAGACGGCAATCTCTTTTTACTGCATCT
CGTCAGTTGGCAACTTGCCAAGAACTTCGCAAATGACTTTGACA
TATGATAAGACGTCAACTGCCCCACGTACAATAACAAAATGGTA
GTCATATTATGTCAAGAATAGGTATCCAAAACGCAGCGGTTGAA
AGCATATCAAGAATTGTGTCCCTGTGTTTCAAAGTTTGTGGATA
ATCGAAATCTCTTACATTGAAAACATTATCATACAATCATTTAT
TAAGTAGTTGAAGCATGTATGAACTATAAAAGTGTTACTACTCG
TTATTATTGTGTACTTTGTGATGCTAAAGTTATGAGTAGAAAAA
AATGAGAAGTTGTTCTGAACAAAGTAAAAAAAAGAAGTATACTT
ATTCAAAATGGGAGAATTGTTGACGCAAAACTCTACGCATGATC
TTGTTGGTGGCAGTTCTAGGCAAAGAAGACAAAGGGACGACTCT
AGTAACCTTAAACAATGGATTCAACTTCTTTTGCAAACCCAAGT
TGAAGGACAATCTCAATTGGTTCAAGTCGATAGTAGTATCGTTA
GAATCCTTCAAGACGAAGAAAATAACCAATTGTTCTGGACCACC
ACCTAATGGTGGAACACCGATAGCAGTGGTTTCGAAAACTCTGT
CATCGACTTCGTTACAAACTCTTTCAATCTCAATGGAAGAGATT
TTGATACCACCGATGTTCATGGTGTCATCAGCACGACCGTGAGC
ATGGTAGTAACCGTTGGAAGTTAATTCAAAGATGTCACCGTGTC
TTCTCAAAACTTCACCGTTCAAAGTTGGCATACCTTTGAAGTAG
ACATCGTGGTGGTTACCGTTCAATAAAGTCTTAGAAGCACCGAA
CATAACTGGACCCAAAGCCAATTCACCAATACCTGGCTTGTTCT
TTGGCATTGGGTAACCGTTCTTATCCAAAATGTACAAAGTACAA
CCCATACATTGGGAAGAAAAGGAGGACAAGGATTGGGCTTGTAA
GAAAGAACCAGCAGAGAAAGCACCACCGATTTCGGTACCACCAC
ACATTTCGATAACAGGTTTATAGTTGGCTCTACCCATCAACCAC
AAGTATTCATCGACGTTAGAAGCTTCACCAGAGGAAGAAAAGCA
ACGGATGGTAGACCAGTCATAACCGGAAACGCAGTTGGTGGATT
TCCAAGATCTAACAATAGATGGAACAACACCTAACATAGTAACC
TTAGCGTCTTGGACGAACTTGGCGAAACCAGAAACCAATGGGGA
ACCATTATACAAAGCGATAGAAGCACCGTTCAATAAAGAGGCGT
AAACCAACCATGGACCCATCATCCAACCTAAATTAGTTGGCCAA
ACAATGACGTCACCTTTACGAATATCCAAGTGAGACCAACCGTC
GGCAGCAGCCTTCAATGGAGTAGCTTGGGTCCATGGAATGGCCT
TTGGTTCACCAGTGGTACCGGAAGAGAATAAAATGTTGGTGTAG
GCATCAACTGGTTGTTCACGAGCGGTGAATTCACAGTTCTTGAA
TTCCTTAGCACGTTCCAAGAAATAATCCCAGGAAATGTCACCGT
CACGCAATTCGGCACCGATGTTGGAACCGGAACATGGAATGACA
ATAGCCATTGGAGACTTAGCTTCAACGACTCTAGAATACAATGG
AATTCTCTTCTTACCACGGATGATGTGGTCTTGAGTGAAGATGG
CCTTAGCCTTAGACAATCTCAATCTAGTAGAGATTTCTGGAGCG
GAGAAAGAATCAGCGATGGAAACGACGACGTAACCAGCCAAGAC
AATGGCTAAGTAGATGACGACAGCGTCAACGTGCATTGGCATAT
CGATGGCGATAGCACAACCTTTTTCCAAACCCATTTCTTCCAAG
GCATAACCAACCAACCAAACTCTCTTTCTCAATTGGTCCAAAGT
CAACTTGTTCAATGGCAAATCGTCGTTACCCTCATCACGCCAAA
CGATCATAGTATCATTCAACTTTTTGTTAGAGTTAACATTCAAG
CAGTTCTTAGCAGAGTTCAAGTAACCACCTGGCAACCATTCGGA
ACCACCTGGGTTGTTAATATCGTCTCTACGTAAGATACATTCTG
GATCTTTAGAAAAGGAGATCTTCATTTCATCCATTAAAACAGTT
CTCCAGTAAACTTCTGGGTTTCTGACGGAGAACTCTTGGAAGTG
AGAAAAAGAAGAAATTGGATCCTTGTATTTAACACCCAAGAATT
CCTTACCTCTTTTCTCCAACAAAGCACCCAAGTTGGTAGACTTG
ACCTTTTCAGGGTCTGGAATCCAAGCTGGTGGGGCTGGACCAAA
GTCCTTGTAACAACCATAGAATAACATTTGGTGCAAGGAAAATG
GCAAGTCTGGGGATAAGATATGGTTGGCAATGTTAATCCAAGTT
TGTGGGGTAGCAGCACCGTAATTACAAACAATTTCAGCTAATCT
ACCATGCAAAGTTTCGGCGACCTCAGAGGTAATACCCAAAGCGA
TGAAATCGGAAGCAACAACAGAATCCAAAGATTTGTAGTTCTTA
CCCATTATAGTTTTTTCTCCTTGACGTTAAAGTATAGAGGTATA
TTAACAATTTTTTGTTGATACTTTTATGACATTTGAATAAGAAG
TAATACAAACCGAAAATGTTGAAAGTATTAGTTAAAGTGGTTAT
GCAGCTTTTGCATTTATATATCTGTTAATAGATCAAAAATCATC
GCTTCGCTGATTAATTACCCCAGAAATAAGGCTAAAAAACTAAT
CGCATTATTATCCTATGGTTGTTAATTTGATTCGTTGATTTGAA
GGTTTGTGGGGCCAGGTTACTGCCAATTTTTCCTCTTCATAACC
ATAAAAGCTAGTATTGTAGAATCTTTATTGTTCGGAGCAGTGCG
GCGCGAGGCACATCTGCGTTTCAGGAACGCGACCGGTGAAGACC
AGGACGCACGGAGGAGAGTCTTCCGTCGGAGGGCTGTCGCCCGC
TCGGCGGCTTCTAATCCGTACTTCAATATAGCAATGAGCAGTTA
AGCGTATTACTGAAAGTTCCAAAGAGAAGGTTTTTTTAGGCTAA
GATAATGGGGCTCTTTACATTTCCACAACATATAAGTAAGATTA
GATATGGATATGTATATGGTGGTATTGCCATGTAATATGATTAT
TAAACTTCTTTGCGTCCATCCAAAAAAAAAGTAAGAATTTTTGA
AAATTCAATATAAATGAACCACTTAAGAGCTGAAGGTCCAGCTT
CCGTTTTGGCCATTGGTACCGCTAACCCAGAAAACATCTTGTTG
CAAGACGAATTTCCAGACTACTACTTCAGAGTCACTAAGTCCGA
ACACATGACCCAATTGAAGGAAAAGTTCAGAAAGATTTGTGATA
AGTCTATGATCAGAAAAAGAAACTGTTTCTTGAACGAAGAACAC
TTGAAACAAAACCCTAGATTAGTTGAACATGAAATGCAAACTTT
AGATGCCAGACAAGATATGTTGGTCGTCGAAGTCCCAAAGTTGG
GTAAGGACGCTTGTGCCAAGGCTATCAAGGAATGGGGTCAACCA
AAGTCTAAGATTACTCATTTGATCTTCACTTCCGCCTCTACCAC
CGATATGCCAGGTGCTGATTACCATTGTGCTAAGTTGTTGGGTT
TATCCCCATCTGTTAAAAGAGTTATGATGTACCAATTGGGTTGT
TATGGTGGTGGTACTGTTTTGAGAATTGCCAAAGACATCGCTGA
AAACAATAAGGGTGCTAGAGTTTTGGCTGTTTGTTGTGATATTA
TGGCTTGTTTGTTCAGAGGTCCATCCGAGTCTGATTTAGAGTTG
TTAGTTGGTCAAGCTATTTTCGGTGACGGTGCTGCTGCTGTTAT
TGTTGGTGCTGAACCAGACGAATCTGTTGGTGAACGTCCAATCT
TTGAATTGGTCTCTACCGGTCAAACCATCTTGCCAAACTCTGAA
GGTACCATTGGTGGTCACATCAGAGAAGCTGGTTTGATCTTCGA
TTTGCATAAAGATGTTCCTATGTTGATTTCTAATAACATCGAAA
AGTGCTTAATCGAAGCTTTCACTCCAATCGGTATCTCTGATTGG
AATTCCATTTTCTGGATTACCCATCCAGGTGGTAAGGCCATCTT
GGATAAGGTTGAAGAAAAGTTGCATTTAAAGTCTGATAAGTTCG
TTGACTCTCGTCACGTTTTGTCTGAACATGGTAACATGTCTTCT
TCCACTGTTTTGTTTGTTATGGATGAATTGAGAAAAAGATCCTT
GGAAGAAGGTAAGTCTACTACTGGTGATGGTTTTGAATGGGGTG
TCTTGTTCGGTTTTGGTCCAGGTTTGACCGTTGAAAGAGTTGTC
GTTAGATCCGTTCCAATCAAGTACTAATTTGCCAGCTTACTATC
CTTCTTGAAAATATGCACTCTATATCTTTTAGTTCTTAATTGCA
ACACATAGATTTGCTGTATAACGAATTTTATGCTATTTTTTAAA
TTTGGAGTTCAGTGATAAAAGTGTCACAGCGAATTTCCTCACAT
GTAGGGACCGAATTGTTTACAAGTTCTCTGTACCACCATGGAGA
CATCAAAGATTGAAAATCTATGGAAAGATATGGACGGTAGCAAC
AAGAATATAGCACGAGCCGCGAAGTTCATTTCGTTACTTTTGAT
ATCGCTCACAACTATTGCGAAGCGCTTCAGTGAAAAAATCATAA
GGAAAAGTTGTAAATATTATTGGTAGTATTCGTTTGGTAAAGTA
GAGGGGGTAATTTTTCCCCTTTATTTTGTTCATACATTCTTAAA
TTGCTTTGCCTCTCCTTTTGGAAAGCTAGGTCCGCCGGCGTTGG
ACGAGCGAAAATTCATTTAATATTCAATGAAGTTATAAATTGAT
AGTTTAAATAAAGTCAGTCTTTTTCCTCATGTTTAGAATTGTAT
TAATGTACGCCGTTTACGTTGGAGTGTAAATGTGTCTATTCCAG
AACGAAATCTAAATGAGCAGTACAGGCAGTTCAGATGGTACTGA
AGCGGTACTAAAGATGCATGAATTGAACAGATGTGGTAGTTATG
TATATGAGGAATATGAGTTGTCACATTAAAAATATAATAGCTAT
GATCCCATTATTATATTCGTGACAGTTCGTAACGTTTTAATTGG
CTTATGTTTTTGAGAAATGGGTGAATTTTAAGATAATTGTTGGG
ATTCCATTATTGATAAAGGCTATAATATTAGGTATACAGAATAT
ACTGGAAGTTCTCCTCGAGGATATAGGAATCCTCAAAATGGAAT
CTATATTTCTATTTACTAATATCACGATTATTCTTCATTCCGTT
TTATATGTTTCATTATCCTATTACATTATCAATCCTTGCATTTC
AGCTTCCTCTAACTTCGATGACAGCTGGCGGTTTAAACGCGTGG
CCGTGCCGTC
MS101227 (SEQ ID NO: 2):
GACGGCACGGCCACGCGTTTAAACCGCCAGAGTATGTCAACTGG
CGCAGTAGATACATGTTTTTCTCTTCCACGTCGAATTTTGTTAT
ATACATAGCATAATCGAGTTGTATGCACCCTTTTTGTTTATCTC
GTTAGTAACTCGGGGTAGGAATAAGACATCCACAAAGGTGACAG
AACAAAATCATCCTAGCCTTGTTCATAATCTACCTCTATATAGC
CGCTAAAAAATTAGTAGTATTTTGACTCTTTAAGAGCACATTTA
TTATCAGGCTGCTTTTACATACTTCTTTTGTTTAAAACATTTAA
AGACGATCACTGCCCTTCCAAAGGACAAATATATATACACAAAC
ACTAGGCCAAAAGTTCACTTATAATAATTTAGTGGTAATTATGT
TGGGTAAAGAAATTGCCAATAGTCTTTTTTTTTCCGTATTGTAA
GGTGAGACTGAGGTAGCGGCACAAAAAAACGACACATAATAGGA
TACTGAGTAAAGCAGTATTAAAATAAAAAGATATATTTTACCTC
GAACGCTACAAATAAAGCAGAAAAGAACAAAATCGTGAGCCGCT
CGTCCAACGCCGGCGGACCTAGCTTTCCAAAAGGAGAGGCAAAG
CAATTTAAGAATGTATGAACAAAATAAAGGGGAAAAATTACCCC
CTCTACTTTACCAAACGAATACTACCAATAATATTTACAACTTT
TCCTTATGATTTTTTCACTGAAGCGCTTCGCAATAGTTGTGAGC
GATATCAAAAGTAACGAAATGAACTTCGCGGCTCGTGCTATATT
CTTGTTGCTACCGTCCATATCTTTCCATAGATTTTCAATCTTTG
ATGTCTCCATGGTGGTACAGAGAACTTGTAAACAATTCGGTCCC
TACATGTGAGGAAATTCGCTGTGACACTTTTATCACTGAACTCC
AAATTTAAAAAATAGCATAAAATTCGTTATACAGCAAATCTATG
TGTTGCAATTAAGAACTAAAAGATATAGAGTGCATATTTTCAAG
AAGGATAGTAAGCTGGCAAATTAGTACTTGATTGGAACGGATCT
AACGACAACTCTTTCAACGGTCAAACCTGGACCAAAACCGAACA
AGACACCCCATTCAAAACCATCACCAGTAGTAGACTTACCTTCT
TCCAAGGATCTTTTTCTCAATTCATCCATAACAAACAAAACAGT
GGAAGAAGACATGTTACCATGTTCAGACAAAACGTGACGAGAGT
CAACGAACTTATCAGACTTTAAATGCAACTTTTCTTCAACCTTA
TCCAAGATGGCCTTACCACCTGGATGGGTAATCCAGAAAATGGA
ATTCCAATCAGAGATACCGATTGGAGTGAAAGCTTCGATTAAGC
ACTTTTCGATGTTATTAGAAATCAACATAGGAACATCTTTATGC
AAATCGAAGATCAAACCAGCTTCTCTGATGTGACCACCAATGGT
ACCTTCAGAGTTTGGCAAGATGGTTTGACCGGTAGAGACCAATT
CAAAGATTGGACGTTCACCAACAGATTCGTCTGGTTCAGCACCA
ACAATAACAGCAGCAGCACCGTCACCGAAAATAGCTTGACCAAC
TAACAACTCTAAATCAGACTCGGATGGACCTCTGAACAAACAAG
CCATAATATCACAACAAACAGCCAAAACTCTAGCACCCTTATTG
TTTTCAGCGATGTCTTTGGCAATTCTCAAAACAGTACCACCACC
ATAACAACCCAATTGGTACATCATAACTCTTTTAACAGATGGGG
ATAAACCCAACAACTTAGCACAATGGTAATCAGCACCTGGCATA
TCGGTGGTAGAGGCGGAAGTGAAGATCAAATGAGTAATCTTAGA
CTTTGGTTGACCCCATTCCTTGATAGCCTTGGCACAAGCGTCCT
TACCCAACTTTGGGACTTCGACGACCAACATATCTTGTCTGGCA
TCTAAAGTTTGCATTTCATGTTCAACTAATCTAGGGTTTTGTTT
CAAGTGTTCTTCGTTCAAGAAACAGTTTCTTTTTCTGATCATAG
ACTTATCACAAATCTTTCTGAACTTTTCCTTCAATTGGGTCATG
TGTTCGGACTTAGTGACTCTGAAGTAGTAGTCTGGAAATTCGTC
TTGCAACAAGATGTTTTCTGGGTTAGCGGTACCAATGGCCAAAA
CGGAAGCTGGACCTTCAGCTCTTAAGTGGTTCATTTATATTGAA
TTTTCAAAAATTCTTACTTTTTTTTTGGATGGACGCAAAGAAGT
TTAATAATCATATTACATGGCAATACCACCATATACATATCCAT
ATCTAATCTTACTTATATGTTGTGGAAATGTAAAGAGCCCCATT
ATCTTAGCCTAAAAAAACCTTCTCTTTGGAACTTTCAGTAATAC
GCTTAACTGCTCATTGCTATATTGAAGTACGGATTAGAAGCCGC
CGAGCGGGCGACAGCCCTCCGACGGAAGACTCTCCTCCGTGCGT
CCTGGTCTTCACCGGTCGCGTTCCTGAAACGCAGATGTGCCTCG
CGCCGCACTGCTCCGAACAATAAAGATTCTACAATACTAGCTTT
TATGGTTATGAAGAGGAAAAATTGGCAGTAACCTGGCCCCACAA
ACCTTCAAATCAACGAATCAAATTAACAACCATAGGATAATAAT
GCGATTAGTTTTTTAGCCTTATTTCTGGGGTAATTAATCAGCGA
AGCGATGATTTTTGATCTATTAACAGATATATAAATGCAAAAGC
TGCATAACCACTTTAACTAATACTTTCAACATTTTCGGTTTGTA
TTACTTCTTATTCAAATGTCATAAAAGTATCAACAAAAAATTGT
TAATATACCTCTATACTTTAACGTCAAGGAGAAAAAACTATAAT
GAACCACTTAAGAGCTGAAGGTCCAGCTTCCGTTTTGGCCATTG
GTACCGCTAACCCAGAAAACATCTTGTTGCAAGACGAATTTCCA
GACTACTACTTCAGAGTCACTAAGTCCGAACACATGACCCAATT
GAAGGAAAAGTTCAGAAAGATTTGTGATAAGTCTATGATCAGAA
AAAGAAACTGTTTCTTGAACGAAGAACACTTGAAACAAAACCCT
AGATTAGTTGAACATGAAATGCAAACTTTAGATGCCAGACAAGA
TATGTTGGTCGTCGAAGTCCCAAAGTTGGGTAAGGACGCTTGTG
CCAAGGCTATCAAGGAATGGGGTCAACCAAAGTCTAAGATTACT
CATTTGATCTTCACTTCCGCCTCTACCACCGATATGCCAGGTGC
TGATTACCATTGTGCTAAGTTGTTGGGTTTATCCCCATCTGTTA
AAAGAGTTATGATGTACCAATTGGGTTGTTATGGTGGTGGTACT
GTTTTGAGAATTGCCAAAGACATCGCTGAAAACAATAAGGGTGC
TAGAGTTTTGGCTGTTTGTTGTGATATTATGGCTTGTTTGTTCA
GAGGTCCATCCGAGTCTGATTTAGAGTTGTTAGTTGGTCAAGCT
ATTTTCGGTGACGGTGCTGCTGCTGTTATTGTTGGTGCTGAACC
AGACGAATCTGTTGGTGAACGTCCAATCTTTGAATTGGTCTCTA
CCGGTCAAACCATCTTGCCAAACTCTGAAGGTACCATTGGTGGT
CACATCAGAGAAGCTGGTTTGATCTTCGATTTGCATAAAGATGT
TCCTATGTTGATTTCTAATAACATCGAAAAGTGCTTAATCGAAG
CTTTCACTCCAATCGGTATCTCTGATTGGAATTCCATTTTCTGG
ATTACCCATCCAGGTGGTAAGGCCATCTTGGATAAGGTTGAAGA
AAAGTTGCATTTAAAGTCTGATAAGTTCGTTGACTCTCGTCACG
TTTTGTCTGAACATGGTAACATGTCTTCTTCCACTGTTTTGTTT
GTTATGGATGAATTGAGAAAAAGATCCTTGGAAGAAGGTAAGTC
TACTACTGGTGATGGTTTTGAATGGGGTGTCTTGTTCGGTTTTG
GTCCAGGTTTGACCGTTGAAAGAGTTGTCGTTAGATCCGTTCCA
ATCAAGTACTAAGTATACTTCTTTTTTTTACTTTGTTCAGAACA
ACTTCTCATTTTTTTCTACTCATAACTTTAGCATCACAAAGTAC
ACAATAATAACGAGTAGTAACACTTTTATAGTTCATACATGCTT
CAACTACTTAATAAATGATTGTATGATAATGTTTTCAATGTAAG
AGATTTCGATTATCCACAAACTTTGAAACACAGGGACACAATTC
TTGATATGCTTTCAACCGCTGCGTTTTGGATACCTATTCTTGAC
ATAATATGACTACCATTTTGTTATTGTACGTGGGGCAGTTGACG
TCTTATCATATGTCAAAGTCATTTGCGAAGTTCTTGGCAAGTTG
CCAACTGACGAGATGCAGTAAAAAGAGATTGCCGTCTTGAAACT
TTTTGTCCTTTTTTTTTTCCGGGGACTCTACGAGAACCCTTTGT
CCTACTGATCCCCGCGTGCTTGGCCGGCCGTGATCATCTACCCA
TGCCGAAATTCGGGCCGTTGGCCGGATTGCGCGTTGTCTTCTCC
GGTATCGAAATCGCCGGACCGTTTGCCGGGCAAATGTTCGCAGA
ATGGGGCGCGGAAGTTATCTGGATCGAGAACGTCGCCTGGGCCG
ACACCATTCGCGTTCAACCGAACTACCCGCAACTCTCCCGCCGC
AATTTGCACGCGCTGTCGTTAAATATTTTCAAAGATGAAGGCCG
CGAAGCGTTTCTGAAATTAATGGAAACCACCGATATCTTCATCG
AAGCCAGTAAAGGTCCGGCCTTTGCCCGTCGTGGCATTACCGAT
GAAGTACTGTGGCAGCACAACCCGAAACTGGTTATCGCTCACCT
GTCCGGTTTTGGTCAGTACGGCACCGAGGAGTACACCAATCTTC
CGGCCTATAACACTATCGCCCAGGCCTTTAGTGGTTACCTGATT
CAGAACGGTGATGTTGACCAGCCAATGCCTGCCTTCCCGTATAC
CGCCGATTACTTTTCTGGCCTGACCGCCACCACGGCGGCGCTGG
CAGCACTGCATAAAGTGCGTGAAACCGGTAAAGGCGAAAGTATC
GACATCGCCATGTATGAAGTGATGCTGCGTATGGGCCAGTACTT
CATGATGGATTACTTCAACGGCGGCGAAATGTGCCCGCGCATGA
GCAAAGGTAAAGATCCCTACTACGCCGAGGTCCGCCGGCGTTGG
ACGAGCGACTTTAATGTCGTTCTCCCTTTTTAAAGAGTAAATAC
ATATTTAAAAAAGTGACTATGGCTATTGCTAAACGTGATAAAAA
TCAGAGCCTATAACACTCTCTGAAATAACGCTATGCAGGAATTT
CCAGTTAAGTTCTTCTTGGGGTGACTTCTTTACTCGGTATGATA
TGTGTTTTATATGCACAGTACGAGTCCATTAGGGTAAATTAGTG
GCCGAGAAACTTTTGCCGCCGAGCTTTTAAGTATCCTTTTGCCA
CTTCTTATTTAGATAAAGACCTGGCAGTAGTAGTCGTAGAAGAT
AAGATAGACAGAGAATGAATACTAATAAGATAGCACAAGACGAA
GTCCAAGATAAGGTTTTGCAAAGAGCAGAACTAGCACATTCTGT
ATGGAACTTAAGGTTCAACCTCAGTAAAGTTGCCAAACGGATTC
GCATGGAAACAAAGGTATTTCCAGAGATAAAGATAAATGACGCG
CAATCACAGTTAGAGCGATCTAGGTGTAGAATATTTAGCCCTGA
CCTGGAGGAAGAACATGTGCCCTTGATTCAAGGCGGCGGTTTAA
ACGCGTGGCCGTGCCGTC
MS101226 (SEQ ID NO: 3):
GACGGCACGGCCACGCGTTTAAACCGCCTACGCCATCATTAAAG
ACCTGGTCAACTATAAAATAATACAATCAATACTTGCTTGAACG
CTTGATTTTACTGATATTCTATCCAAAAGCAAGTAGACCAGAAA
CTCTCAAGATGTTGCAAATACCGTTCGATGTTTTTGGTTTAGAT
TGTTTTAATGTTGATGCTTTTTTACTTATTTTTGGAAGCGTCTT
TTTAATTTAGTTTTATATTATAGGTATATGAATGTGTTTATGCC
AATAAGGGTTTTTTTGTACAGTTATGTGATTATAAACAGTCTTT
TGTCTAGTTTTTTTCACCAGTATCGGCCTCTATTTATAAAAAAC
GGAGCAGCTTTCGGTGTCAGTAATTCTGAAAAAATTTGTGTCAC
TCTGATTGTAAATGAATTAATTTAGCTAGATAGTTGCGAGCCCC
AACGAGAAGATTGTCAGACAAAGACAACATTCAACAACCTACAT
CCGTTACTATTCGTTAACTCGAGGTACTTGAAACTTTTCAGTTA
AGTCGCTCGTCCAACGCCGGCGGACCTGCGAGTAAGCAACTCTG
GCGCTGGCATGGCATAACCGGCGACGGCAATGCGCAAGATGGGA
TGCTATGGGCAGAGAGCCGTACTTTACTGCTTATGGCACTACAG
CAACAGATGGTTACCCCACTAAGCCTGAAGCGAATCGCCATCAA
TTCTGCGCAGTGGCGAGGAGATAAAAGCGCGGAAGTCATTCATC
AACTGGCGACGCTACTCAAAGCAGGGTTAACGCTTTCTGAAGGG
CTGGCTCTGCTGGCGGAACAGCATCCCAGTAAGCAATGGCAAGC
GTTGCTGCAATCGCTGGCGCACGATCTCGAACAGGGCATTGCTT
TTTCCAATGCCTTATTACCCTGGTCAGAGGTATTTCCGCCGCTC
TATCAGGCGATGATCCGCACGGGTGAACTGACCGGTAAGCTGGA
TGAATGCTGCTTTGAACTGGCGCGTCAGCAAAAAGCCCAGCGTC
AGTTGACCGACAAAGTGAAATCAGCGTTACGTTATCCCATCATC
ATTTTAGCGATGGCAATCATGGTGGTTGTGGCAATGCTGCATTT
TGTTCTGCCGGAGTTTGCCGCTATCTATAAGACCTTCAACACCC
CACTACCGGCACTAACGCAGGGGATCATGACGCTGGCAGACTTT
AGTGGCGAATGGAGCTGGCTGCTGGTGTTGTTCGGCTTTCTGCT
GGCGATAGCCAATAAGTTGCTGAACGGCCGGCCAAGCACGCGGG
GATCAGTAGGACAAAGGGTTCTCGTAGAGTCCCCGGAAAAAAAA
AAGGACAAAAAGTTTCAAGACGGCAATCTCTTTTTACTGCATCT
CGTCAGTTGGCAACTTGCCAAGAACTTCGCAAATGACTTTGACA
TATGATAAGACGTCAACTGCCCCACGTACAATAACAAAATGGTA
GTCATATTATGTCAAGAATAGGTATCCAAAACGCAGCGGTTGAA
AGCATATCAAGAATTGTGTCCCTGTGTTTCAAAGTTTGTGGATA
ATCGAAATCTCTTACATTGAAAACATTATCATACAATCATTTAT
TAAGTAGTTGAAGCATGTATGAACTATAAAAGTGTTACTACTCG
TTATTATTGTGTACTTTGTGATGCTAAAGTTATGAGTAGAAAAA
AATGAGAAGTTGTTCTGAACAAAGTAAAAAAAAGAAGTATACTT
AGTACTTGATTGGAACGGATCTAACGACAACTCTTTCAACGGTC
AAACCTGGACCAAAACCGAACAAGACACCCCATTCAAAACCATC
ACCAGTAGTAGACTTACCTTCTTCCAAGGATCTTTTTCTCAATT
CATCCATAACAAACAAAACAGTGGAAGAAGACATGTTACCATGT
TCAGACAAAACGTGACGAGAGTCAACGAACTTATCAGACTTTAA
ATGCAACTTTTCTTCAACCTTATCCAAGATGGCCTTACCACCTG
GATGGGTAATCCAGAAAATGGAATTCCAATCAGAGATACCGATT
GGAGTGAAAGCTTCGATTAAGCACTTTTCGATGTTATTAGAAAT
CAACATAGGAACATCTTTATGCAAATCGAAGATCAAACCAGCTT
CTCTGATGTGACCACCAATGGTACCTTCAGAGTTTGGCAAGATG
GTTTGACCGGTAGAGACCAATTCAAAGATTGGACGTTCACCAAC
AGATTCGTCTGGTTCAGCACCAACAATAACAGCAGCAGCACCGT
CACCGAAAATAGCTTGACCAACTAACAACTCTAAATCAGACTCG
GATGGACCTCTGAACAAACAAGCCATAATATCACAACAAACAGC
CAAAACTCTAGCACCCTTATTGTTTTCAGCGATGTCTTTGGCAA
TTCTCAAAACAGTACCACCACCATAACAACCCAATTGGTACATC
ATAACTCTTTTAACAGATGGGGATAAACCCAACAACTTAGCACA
ATGGTAATCAGCACCTGGCATATCGGTGGTAGAGGCGGAAGTGA
AGATCAAATGAGTAATCTTAGACTTTGGTTGACCCCATTCCTTG
ATAGCCTTGGCACAAGCGTCCTTACCCAACTTTGGGACTTCGAC
GACCAACATATCTTGTCTGGCATCTAAAGTTTGCATTTCATGTT
CAACTAATCTAGGGTTTTGTTTCAAGTGTTCTTCGTTCAAGAAA
CAGTTTCTTTTTCTGATCATAGACTTATCACAAATCTTTCTGAA
CTTTTCCTTCAATTGGGTCATGTGTTCGGACTTAGTGACTCTGA
AGTAGTAGTCTGGAAATTCGTCTTGCAACAAGATGTTTTCTGGG
TTAGCGGTACCAATGGCCAAAACGGAAGCTGGACCTTCAGCTCT
TAAGTGGTTCATTATAGTTTTTTCTCCTTGACGTTAAAGTATAG
AGGTATATTAACAATTTTTTGTTGATACTTTTATGACATTTGAA
TAAGAAGTAATACAAACCGAAAATGTTGAAAGTATTAGTTAAAG
TGGTTATGCAGCTTTTGCATTTATATATCTGTTAATAGATCAAA
AATCATCGCTTCGCTGATTAATTACCCCAGAAATAAGGCTAAAA
AACTAATCGCATTATTATCCTATGGTTGTTAATTTGATTCGTTG
ATTTGAAGGTTTGTGGGGCCAGGTTACTGCCAATTTTTCCTCTT
CATAACCATAAAAGCTAGTATTGTAGAATCTTTATTGTTCGGAG
CAGTGCGGCGCGAGGCACATCTGCGTTTCAGGAACGCGACCGGT
GAAGACCAGGACGCACGGAGGAGAGTCTTCCGTCGGAGGGCTGT
CGCCCGCTCGGCGGCTTCTAATCCGTACTTCAATATAGCAATGA
GCAGTTAAGCGTATTACTGAAAGTTCCAAAGAGAAGGTTTTTTT
AGGCTAAGATAATGGGGCTCTTTACATTTCCACAACATATAAGT
AAGATTAGATATGGATATGTATATGGTGGTATTGCCATGTAATA
TGATTATTAAACTTCTTTGCGTCCATCCAAAAAAAAAGTAAGAA
TTTTTGAAAATTCAATATAAATGAACCACTTAAGAGCTGAAGGT
CCAGCTTCCGTTTTGGCCATTGGTACCGCTAACCCAGAAAACAT
CTTGTTGCAAGACGAATTTCCAGACTACTACTTCAGAGTCACTA
AGTCCGAACACATGACCCAATTGAAGGAAAAGTTCAGAAAGATT
TGTGATAAGTCTATGATCAGAAAAAGAAACTGTTTCTTGAACGA
AGAACACTTGAAACAAAACCCTAGATTAGTTGAACATGAAATGC
AAACTTTAGATGCCAGACAAGATATGTTGGTCGTCGAAGTCCCA
AAGTTGGGTAAGGACGCTTGTGCCAAGGCTATCAAGGAATGGGG
TCAACCAAAGTCTAAGATTACTCATTTGATCTTCACTTCCGCCT
CTACCACCGATATGCCAGGTGCTGATTACCATTGTGCTAAGTTG
TTGGGTTTATCCCCATCTGTTAAAAGAGTTATGATGTACCAATT
GGGTTGTTATGGTGGTGGTACTGTTTTGAGAATTGCCAAAGACA
TCGCTGAAAACAATAAGGGTGCTAGAGTTTTGGCTGTTTGTTGT
GATATTATGGCTTGTTTGTTCAGAGGTCCATCCGAGTCTGATTT
AGAGTTGTTAGTTGGTCAAGCTATTTTCGGTGACGGTGCTGCTG
CTGTTATTGTTGGTGCTGAACCAGACGAATCTGTTGGTGAACGT
CCAATCTTTGAATTGGTCTCTACCGGTCAAACCATCTTGCCAAA
CTCTGAAGGTACCATTGGTGGTCACATCAGAGAAGCTGGTTTGA
TCTTCGATTTGCATAAAGATGTTCCTATGTTGATTTCTAATAAC
ATCGAAAAGTGCTTAATCGAAGCTTTCACTCCAATCGGTATCTC
TGATTGGAATTCCATTTTCTGGATTACCCATCCAGGTGGTAAGG
CCATCTTGGATAAGGTTGAAGAAAAGTTGCATTTAAAGTCTGAT
AAGTTCGTTGACTCTCGTCACGTTTTGTCTGAACATGGTAACAT
GTCTTCTTCCACTGTTTTGTTTGTTATGGATGAATTGAGAAAAA
GATCCTTGGAAGAAGGTAAGTCTACTACTGGTGATGGTTTTGAA
TGGGGTGTCTTGTTCGGTTTTGGTCCAGGTTTGACCGTTGAAAG
AGTTGTCGTTAGATCCGTTCCAATCAAGTACTAATTTGCCAGCT
TACTATCCTTCTTGAAAATATGCACTCTATATCTTTTAGTTCTT
AATTGCAACACATAGATTTGCTGTATAACGAATTTTATGCTATT
TTTTAAATTTGGAGTTCAGTGATAAAAGTGTCACAGCGAATTTC
CTCACATGTAGGGACCGAATTGTTTACAAGTTCTCTGTACCACC
ATGGAGACATCAAAGATTGAAAATCTATGGAAAGATATGGACGG
TAGCAACAAGAATATAGCACGAGCCGCGAAGTTCATTTCGTTAC
TTTTGATATCGCTCACAACTATTGCGAAGCGCTTCAGTGAAAAA
ATCATAAGGAAAAGTTGTAAATATTATTGGTAGTATTCGTTTGG
TAAAGTAGAGGGGGTAATTTTTCCCCTTTATTTTGTTCATACAT
TCTTAAATTGCTTTGCCTCTCCTTTTGGAAAGCTAGGTCCGCCG
GCGTTGGACGAGCGAAAATTCATTTAATATTCAATGAAGTTATA
AATTGATAGTTTAAATAAAGTCAGTCTTTTTCCTCATGTTTAGA
ATTGTATTAATGTACGCCGTTTACGTTGGAGTGTAAATGTGTCT
ATTCCAGAACGAAATCTAAATGAGCAGTACAGGCAGTTCAGATG
GTACTGAAGCGGTACTAAAGATGCATGAATTGAACAGATGTGGT
AGTTATGTATATGAGGAATATGAGTTGTCACATTAAAAATATAA
TAGCTATGATCCCATTATTATATTCGTGACAGTTCGTAACGTTT
TAATTGGCTTATGTTTTTGAGAAATGGGTGAATTTTAAGATAAT
TGTTGGGATTCCATTATTGATAAAGGCTATAATATTAGGTATAC
AGAATATACTGGAAGTTCTCCTCGAGGATATAGGAATCCTCAAA
ATGGAATCTATATTTCTATTTACTAATATCACGATTATTCTTCA
TTCCGTTTTATATGTTTCATTATCCTATTACATTATCAATCCTT
GCATTTCAGCTTCCTCTAACTTCGATGACAGCTGGCGGTTTAAA
CGCGTGGCCGTGCCGTC
MS96695 (SEQ ID NO: 4):
GACGGCACGGCCACGCGTTTAAACCGCCAGAGTATGTCAACTGG
CGCAGTAGATACATGTTTTTCTCTTCCACGTCGAATTTTGTTAT
ATACATAGCATAATCGAGTTGTATGCACCCTTTTTGTTTATCTC
GTTAGTAACTCGGGGTAGGAATAAGACATCCACAAAGGTGACAG
AACAAAATCATCCTAGCCTTGTTCATAATCTACCTCTATATAGC
CGCTAAAAAATTAGTAGTATTTTGACTCTTTAAGAGCACATTTA
TTATCAGGCTGCTTTTACATACTTCTTTTGTTTAAAACATTTAA
AGACGATCACTGCCCTTCCAAAGGACAAATATATATACACAAAC
ACTAGGCCAAAAGTTCACTTATAATAATTTAGTGGTAATTATGT
TGGGTAAAGAAATTGCCAATAGTCTTTTTTTTTCCGTATTGTAA
GGTGAGACTGAGGTAGCGGCACAAAAAAACGACACATAATAGGA
TACTGAGTAAAGCAGTATTAAAATAAAAAGATATATTTTACCTC
GAACGCTACAAATAAAGCAGAAAAGAACAAAATCGTGAGCCGCT
CGTCCAACGCCGGCGGACCTAGCTTTCCAAAAGGAGAGGCAAAG
CAATTTAAGAATGTATGAACAAAATAAAGGGGAAAAATTACCCC
CTCTACTTTACCAAACGAATACTACCAATAATATTTACAACTTT
TCCTTATGATTTTTTCACTGAAGCGCTTCGCAATAGTTGTGAGC
GATATCAAAAGTAACGAAATGAACTTCGCGGCTCGTGCTATATT
CTTGTTGCTACCGTCCATATCTTTCCATAGATTTTCAATCTTTG
ATGTCTCCATGGTGGTACAGAGAACTTGTAAACAATTCGGTCCC
TACATGTGAGGAAATTCGCTGTGACACTTTTATCACTGAACTCC
AAATTTAAAAAATAGCATAAAATTCGTTATACAGCAAATCTATG
TGTTGCAATTAAGAACTAAAAGATATAGAGTGCATATTTTCAAG
AAGGATAGTAAGCTGGCAAATTATTCAAAATGGGAGAATTGTTG
ACGCAAAACTCTACGCATGATCTTGTTGGTGGCAGTTCTAGGCA
AAGAAGACAAAGGGACGACTCTAGTAACCTTAAACAATGGATTC
AACTTCTTTTGCAAACCCAAGTTGAAGGACAATCTCAATTGGTT
CAAGTCGATAGTAGTATCGTTAGAATCCTTCAAGACGAAGAAAA
TAACCAATTGTTCTGGACCACCACCTAATGGTGGAACACCGATA
GCAGTGGTTTCGAAAACTCTGTCATCGACTTCGTTACAAACTCT
TTCAATCTCAATGGAAGAGATTTTGATACCACCGATGTTCATGG
TGTCATCAGCACGACCGTGAGCATGGTAGTAACCGTTGGAAGTT
AATTCAAAGATGTCACCGTGTCTTCTCAAAACTTCACCGTTCAA
AGTTGGCATACCTTTGAAGTAGACATCGTGGTGGTTACCGTTCA
ATAAAGTCTTAGAAGCACCGAACATAACTGGACCCAAAGCCAAT
TCACCAATACCTGGCTTGTTCTTTGGCATTGGGTAACCGTTCTT
ATCCAAAATGTACAAAGTACAACCCATACATTGGGAAGAAAAGG
AGGACAAGGATTGGGCTTGTAAGAAAGAACCAGCAGAGAAAGCA
CCACCGATTTCGGTACCACCACACATTTCGATAACAGGTTTATA
GTTGGCTCTACCCATCAACCACAAGTATTCATCGACGTTAGAAG
CTTCACCAGAGGAAGAAAAGCAACGGATGGTAGACCAGTCATAA
CCGGAAACGCAGTTGGTGGATTTCCAAGATCTAACAATAGATGG
AACAACACCTAACATAGTAACCTTAGCGTCTTGGACGAACTTGG
CGAAACCAGAAACCAATGGGGAACCATTATACAAAGCGATAGAA
GCACCGTTCAATAAAGAGGCGTAAACCAACCATGGACCCATCAT
CCAACCTAAATTAGTTGGCCAAACAATGACGTCACCTTTACGAA
TATCCAAGTGAGACCAACCGTCGGCAGCAGCCTTCAATGGAGTA
GCTTGGGTCCATGGAATGGCCTTTGGTTCACCAGTGGTACCGGA
AGAGAATAAAATGTTGGTGTAGGCATCAACTGGTTGTTCACGAG
CGGTGAATTCACAGTTCTTGAATTCCTTAGCACGTTCCAAGAAA
TAATCCCAGGAAATGTCACCGTCACGCAATTCGGCACCGATGTT
GGAACCGGAACATGGAATGACAATAGCCATTGGAGACTTAGCTT
CAACGACTCTAGAATACAATGGAATTCTCTTCTTACCACGGATG
ATGTGGTCTTGAGTGAAGATGGCCTTAGCCTTAGACAATCTCAA
TCTAGTAGAGATTTCTGGAGCGGAGAAAGAATCAGCGATGGAAA
CGACGACGTAACCAGCCAAGACAATGGCTAAGTAGATGACGACA
GCGTCAACGTGCATTGGCATATCGATGGCGATAGCACAACCTTT
TTCCAAACCCATTTCTTCCAAGGCATAACCAACCAACCAAACTC
TCTTTCTCAATTGGTCCAAAGTCAACTTGTTCAATGGCAAATCG
TCGTTACCCTCATCACGCCAAACGATCATAGTATCATTCAACTT
TTTGTTAGAGTTAACATTCAAGCAGTTCTTAGCAGAGTTCAAGT
AACCACCTGGCAACCATTCGGAACCACCTGGGTTGTTAATATCG
TCTCTACGTAAGATACATTCTGGATCTTTAGAAAAGGAGATCTT
CATTTCATCCATTAAAACAGTTCTCCAGTAAACTTCTGGGTTTC
TGACGGAGAACTCTTGGAAGTGAGAAAAAGAAGAAATTGGATCC
TTGTATTTAACACCCAAGAATTCCTTACCTCTTTTCTCCAACAA
AGCACCCAAGTTGGTAGACTTGACCTTTTCAGGGTCTGGAATCC
AAGCTGGTGGGGCTGGACCAAAGTCCTTGTAACAACCATAGAAT
AACATTTGGTGCAAGGAAAATGGCAAGTCTGGGGATAAGATATG
GTTGGCAATGTTAATCCAAGTTTGTGGGGTAGCAGCACCGTAAT
TACAAACAATTTCAGCTAATCTACCATGCAAAGTTTCGGCGACC
TCAGAGGTAATACCCAAAGCGATGAAATCGGAAGCAACAACAGA
ATCCAAAGATTTGTAGTTCTTACCCATTTATATTGAATTTTCAA
AAATTCTTACTTTTTTTTTGGATGGACGCAAAGAAGTTTAATAA
TCATATTACATGGCAATACCACCATATACATATCCATATCTAAT
CTTACTTATATGTTGTGGAAATGTAAAGAGCCCCATTATCTTAG
CCTAAAAAAACCTTCTCTTTGGAACTTTCAGTAATACGCTTAAC
TGCTCATTGCTATATTGAAGTACGGATTAGAAGCCGCCGAGCGG
GCGACAGCCCTCCGACGGAAGACTCTCCTCCGTGCGTCCTGGTC
TTCACCGGTCGCGTTCCTGAAACGCAGATGTGCCTCGCGCCGCA
CTGCTCCGAACAATAAAGATTCTACAATACTAGCTTTTATGGTT
ATGAAGAGGAAAAATTGGCAGTAACCTGGCCCCACAAACCTTCA
AATCAACGAATCAAATTAACAACCATAGGATAATAATGCGATTA
GTTTTTTAGCCTTATTTCTGGGGTAATTAATCAGCGAAGCGATG
ATTTTTGATCTATTAACAGATATATAAATGCAAAAGCTGCATAA
CCACTTTAACTAATACTTTCAACATTTTCGGTTTGTATTACTTC
TTATTCAAATGTCATAAAAGTATCAACAAAAAATTGTTAATATA
CCTCTATACTTTAACGTCAAGGAGAAAAAACTATAATGAACCAC
TTAAGAGCTGAAGGTCCAGCTTCCGTTTTGGCCATTGGTACCGC
TAACCCAGAAAACATCTTGTTGCAAGACGAATTTCCAGACTACT
ACTTCAGAGTCACTAAGTCCGAACACATGACCCAATTGAAGGAA
AAGTTCAGAAAGATTTGTGATAAGTCTATGATCAGAAAAAGAAA
CTGTTTCTTGAACGAAGAACACTTGAAACAAAACCCTAGATTAG
TTGAACATGAAATGCAAACTTTAGATGCCAGACAAGATATGTTG
GTCGTCGAAGTCCCAAAGTTGGGTAAGGACGCTTGTGCCAAGGC
TATCAAGGAATGGGGTCAACCAAAGTCTAAGATTACTCATTTGA
TCTTCACTTCCGCCTCTACCACCGATATGCCAGGTGCTGATTAC
CATTGTGCTAAGTTGTTGGGTTTATCCCCATCTGTTAAAAGAGT
TATGATGTACCAATTGGGTTGTTATGGTGGTGGTACTGTTTTGA
GAATTGCCAAAGACATCGCTGAAAACAATAAGGGTGCTAGAGTT
TTGGCTGTTTGTTGTGATATTATGGCTTGTTTGTTCAGAGGTCC
ATCCGAGTCTGATTTAGAGTTGTTAGTTGGTCAAGCTATTTTCG
GTGACGGTGCTGCTGCTGTTATTGTTGGTGCTGAACCAGACGAA
TCTGTTGGTGAACGTCCAATCTTTGAATTGGTCTCTACCGGTCA
AACCATCTTGCCAAACTCTGAAGGTACCATTGGTGGTCACATCA
GAGAAGCTGGTTTGATCTTCGATTTGCATAAAGATGTTCCTATG
TTGATTTCTAATAACATCGAAAAGTGCTTAATCGAAGCTTTCAC
TCCAATCGGTATCTCTGATTGGAATTCCATTTTCTGGATTACCC
ATCCAGGTGGTAAGGCCATCTTGGATAAGGTTGAAGAAAAGTTG
CATTTAAAGTCTGATAAGTTCGTTGACTCTCGTCACGTTTTGTC
TGAACATGGTAACATGTCTTCTTCCACTGTTTTGTTTGTTATGG
ATGAATTGAGAAAAAGATCCTTGGAAGAAGGTAAGTCTACTACT
GGTGATGGTTTTGAATGGGGTGTCTTGTTCGGTTTTGGTCCAGG
TTTGACCGTTGAAAGAGTTGTCGTTAGATCCGTTCCAATCAAGT
ACTAAGTATACTTCTTTTTTTTACTTTGTTCAGAACAACTTCTC
ATTTTTTTCTACTCATAACTTTAGCATCACAAAGTACACAATAA
TAACGAGTAGTAACACTTTTATAGTTCATACATGCTTCAACTAC
TTAATAAATGATTGTATGATAATGTTTTCAATGTAAGAGATTTC
GATTATCCACAAACTTTGAAACACAGGGACACAATTCTTGATAT
GCTTTCAACCGCTGCGTTTTGGATACCTATTCTTGACATAATAT
GACTACCATTTTGTTATTGTACGTGGGGCAGTTGACGTCTTATC
ATATGTCAAAGTCATTTGCGAAGTTCTTGGCAAGTTGCCAACTG
ACGAGATGCAGTAAAAAGAGATTGCCGTCTTGAAACTTTTTGTC
CTTTTTTTTTTCCGGGGACTCTACGAGAACCCTTTGTCCTACTG
ATCCCCGCGTGCTTGGCCGGCCGTGATCATCTACCCATGCCGAA
ATTCGGGCCGTTGGCCGGATTGCGCGTTGTCTTCTCCGGTATCG
AAATCGCCGGACCGTTTGCCGGGCAAATGTTCGCAGAATGGGGC
GCGGAAGTTATCTGGATCGAGAACGTCGCCTGGGCCGACACCAT
TCGCGTTCAACCGAACTACCCGCAACTCTCCCGCCGCAATTTGC
ACGCGCTGTCGTTAAATATTTTCAAAGATGAAGGCCGCGAAGCG
TTTCTGAAATTAATGGAAACCACCGATATCTTCATCGAAGCCAG
TAAAGGTCCGGCCTTTGCCCGTCGTGGCATTACCGATGAAGTAC
TGTGGCAGCACAACCCGAAACTGGTTATCGCTCACCTGTCCGGT
TTTGGTCAGTACGGCACCGAGGAGTACACCAATCTTCCGGCCTA
TAACACTATCGCCCAGGCCTTTAGTGGTTACCTGATTCAGAACG
GTGATGTTGACCAGCCAATGCCTGCCTTCCCGTATACCGCCGAT
TACTTTTCTGGCCTGACCGCCACCACGGCGGCGCTGGCAGCACT
GCATAAAGTGCGTGAAACCGGTAAAGGCGAAAGTATCGACATCG
CCATGTATGAAGTGATGCTGCGTATGGGCCAGTACTTCATGATG
GATTACTTCAACGGCGGCGAAATGTGCCCGCGCATGAGCAAAGG
TAAAGATCCCTACTACGCCGAGGTCCGCCGGCGTTGGACGAGCG
ACTTTAATGTCGTTCTCCCTTTTTAAAGAGTAAATACATATTTA
AAAAAGTGACTATGGCTATTGCTAAACGTGATAAAAATCAGAGC
CTATAACACTCTCTGAAATAACGCTATGCAGGAATTTCCAGTTA
AGTTCTTCTTGGGGTGACTTCTTTACTCGGTATGATATGTGTTT
TATATGCACAGTACGAGTCCATTAGGGTAAATTAGTGGCCGAGA
AACTTTTGCCGCCGAGCTTTTAAGTATCCTTTTGCCACTTCTTA
TTTAGATAAAGACCTGGCAGTAGTAGTCGTAGAAGATAAGATAG
ACAGAGAATGAATACTAATAAGATAGCACAAGACGAAGTCCAAG
ATAAGGTTTTGCAAAGAGCAGAACTAGCACATTCTGTATGGAAC
TTAAGGTTCAACCTCAGTAAAGTTGCCAAACGGATTCGCATGGA
AACAAAGGTATTTCCAGAGATAAAGATAAATGACGCGCAATCAC
AGTTAGAGCGATCTAGGTGTAGAATATTTAGCCCTGACCTGGAG
GAAGAACATGTGCCCTTGATTCAAGGCGGCGGTTTAAACGCGTG
GCCGTGCCGTC
MS101224 (SEQ ID NO: 5):
GACGGCACGGCCACGCGTTTAAACCGCCAGAGTATGTCAACTGGC
GCAGTAGATACATGTTTTTCTCTTCCACGTCGAATTTTGTTATA
TACATAGCATAATCGAGTTGTATGCACCCTTTTTGTTTATCTCG
TTAGTAACTCGGGGTAGGAATAAGACATCCACAAAGGTGACAGA
ACAAAATCATCCTAGCCTTGTTCATAATCTACCTCTATATAGCC
GCTAAAAAATTAGTAGTATTTTGACTCTTTAAGAGCACATTTAT
TATCAGGCTGCTTTTACATACTTCTTTTGTTTAAAACATTTAAA
GACGATCACTGCCCTTCCAAAGGACAAATATATATACACAAACA
CTAGGCCAAAAGTTCACTTATAATAATTTAGTGGTAATTATGTT
GGGTAAAGAAATTGCCAATAGTCTTTTTTTTTCCGTATTGTAAG
GTGAGACTGAGGTAGCGGCACAAAAAAACGACACATAATAGGAT
ACTGAGTAAAGCAGTATTAAAATAAAAAGATATATTTTACCTCG
AACGCTACAAATAAAGCAGAAAAGAACAAAATCGTGAGCCGCTC
GTCCAACGCCGGCGGACCTAGCTTTCCAAAAGGAGAGGCAAAGC
AATTTAAGAATGTATGAACAAAATAAAGGGGAAAAATTACCCCC
TCTACTTTACCAAACGAATACTACCAATAATATTTACAACTTTT
CCTTATGATTTTTTCACTGAAGCGCTTCGCAATAGTTGTGAGCG
ATATCAAAAGTAACGAAATGAACTTCGCGGCTCGTGCTATATTC
TTGTTGCTACCGTCCATATCTTTCCATAGATTTTCAATCTTTGA
TGTCTCCATGGTGGTACAGAGAACTTGTAAACAATTCGGTCCCT
ACATGTGAGGAAATTCGCTGTGACACTTTTATCACTGAACTCCA
AATTTAAAAAATAGCATAAAATTCGTTATACAGCAAATCTATGT
GTTGCAATTAAGAACTAAAAGATATAGAGTGCATATTTTCAAGA
AGGATAGTAAGCTGGCAAATTATTCAAAATGGGAGAATTGTTGA
CGCAAAACTCTACGCATGATCTTGTTGGTGGCAGTTCTAGGCAA
AGAAGACAAAGGGACGACTCTAGTAACCTTAAACAATGGATTCA
ACTTCTTTTGCAAACCCAAGTTGAAGGACAATCTCAATTGGTTC
AAGTCGATAGTAGTATCGTTAGAATCCTTCAAGACGAAGAAAAT
AACCAATTGTTCTGGACCACCACCTAATGGTGGAACACCGATAG
CAGTGGTTTCGAAAACTCTGTCATCGACTTCGTTACAAACTCTT
TCAATCTCAATGGAAGAGATTTTGATACCACCGATGTTCATGGT
GTCATCAGCACGACCGTGAGCATGGTAGTAACCGTTGGAAGTTA
ATTCAAAGATGTCACCGTGTCTTCTCAAAACTTCACCGTTCAAA
GTTGGCATACCTTTGAAGTAGACATCGTGGTGGTTACCGTTCAA
TAAAGTCTTAGAAGCACCGAACATAACTGGACCCAAAGCCAATT
CACCAATACCTGGCTTGTTCTTTGGCATTGGGTAACCGTTCTTA
TCCAAAATGTACAAAGTACAACCCATACATTGGGAAGAAAAGGA
GGACAAGGATTGGGCTTGTAAGAAAGAACCAGCAGAGAAAGCAC
CACCGATTTCGGTACCACCACACATTTCGATAACAGGTTTATAG
TTGGCTCTACCCATCAACCACAAGTATTCATCGACGTTAGAAGC
TTCACCAGAGGAAGAAAAGCAACGGATGGTAGACCAGTCATAAC
CGGAAACGCAGTTGGTGGATTTCCAAGATCTAACAATAGATGGA
ACAACACCTAACATAGTAACCTTAGCGTCTTGGACGAACTTGGC
GAAACCAGAAACCAATGGGGAACCATTATACAAAGCGATAGAAG
CACCGTTCAATAAAGAGGCGTAAACCAACCATGGACCCATCATC
CAACCTAAATTAGTTGGCCAAACAATGACGTCACCTTTACGAAT
ATCCAAGTGAGACCAACCGTCGGCAGCAGCCTTCAATGGAGTAG
CTTGGGTCCATGGAATGGCCTTTGGTTCACCAGTGGTACCGGAA
GAGAATAAAATGTTGGTGTAGGCATCAACTGGTTGTTCACGAGC
GGTGAATTCACAGTTCTTGAATTCCTTAGCACGTTCCAAGAAAT
AATCCCAGGAAATGTCACCGTCACGCAATTCGGCACCGATGTTG
GAACCGGAACATGGAATGACAATAGCCATTGGAGACTTAGCTTC
AACGACTCTAGAATACAATGGAATTCTCTTCTTACCACGGATGA
TGTGGTCTTGAGTGAAGATGGCCTTAGCCTTAGACAATCTCAAT
CTAGTAGAGATTTCTGGAGCGGAGAAAGAATCAGCGATGGAAAC
GACGACGTAACCAGCCAAGACAATGGCTAAGTAGATGACGACAG
CGTCAACGTGCATTGGCATATCGATGGCGATAGCACAACCTTTT
TCCAAACCCATTTCTTCCAAGGCATAACCAACCAACCAAACTCT
CTTTCTCAATTGGTCCAAAGTCAACTTGTTCAATGGCAAATCGT
CGTTACCCTCATCACGCCAAACGATCATAGTATCATTCAACTTT
TTGTTAGAGTTAACATTCAAGCAGTTCTTAGCAGAGTTCAAGTA
ACCACCTGGCAACCATTCGGAACCACCTGGGTTGTTAATATCGT
CTCTACGTAAGATACATTCTGGATCTTTAGAAAAGGAGATCTTC
ATTTCATCCATTAAAACAGTTCTCCAGTAAACTTCTGGGTTTCT
GACGGAGAACTCTTGGAAGTGAGAAAAAGAAGAAATTGGATCCT
TGTATTTAACACCCAAGAATTCCTTACCTCTTTTCTCCAACAAA
GCACCCAAGTTGGTAGACTTGACCTTTTCAGGGTCTGGAATCCA
AGCTGGTGGGGCTGGACCAAAGTCCTTGTAACAACCATAGAATA
ACATTTGGTGCAAGGAAAATGGCAAGTCTGGGGATAAGATATGG
TTGGCAATGTTAATCCAAGTTTGTGGGGTAGCAGCACCGTAATT
ACAAACAATTTCAGCTAATCTACCATGCAAAGTTTCGGCGACCT
CAGAGGTAATACCCAAAGCGATGAAATCGGAAGCAACAACAGAA
TCCAAAGATTTGTAGTTCTTACCCATTTATATTGAATTTTCAAA
AATTCTTACTTTTTTTTTGGATGGACGCAAAGAAGTTTAATAAT
CATATTACATGGCAATACCACCATATACATATCCATATCTAATC
TTACTTATATGTTGTGGAAATGTAAAGAGCCCCATTATCTTAGC
CTAAAAAAACCTTCTCTTTGGAACTTTCAGTAATACGCTTAACT
GCTCATTGCTATATTGAAGTACGGATTAGAAGCCGCCGAGCGGG
CGACAGCCCTCCGACGGAAGACTCTCCTCCGTGCGTCCTGGTCT
TCACCGGTCGCGTTCCTGAAACGCAGATGTGCCTCGCGCCGCAC
TGCTCCGAACAATAAAGATTCTACAATACTAGCTTTTATGGTTA
TGAAGAGGAAAAATTGGCAGTAACCTGGCCCCACAAACCTTCAA
ATCAACGAATCAAATTAACAACCATAGGATAATAATGCGATTAG
TTTTTTAGCCTTATTTCTGGGGTAATTAATCAGCGAAGCGATGA
TTTTTGATCTATTAACAGATATATAAATGCAAAAGCTGCATAAC
CACTTTAACTAATACTTTCAACATTTTCGGTTTGTATTACTTCT
TATTCAAATGTCATAAAAGTATCAACAAAAAATTGTTAATATAC
CTCTATACTTTAACGTCAAGGAGAAAAAACTATAATGAACCACT
TAAGAGCTGAAGGTCCAGCTTCCGTTTTGGCCATTGGTACCGCT
AACCCAGAAAACATCTTGTTGCAAGACGAATTTCCAGACTACTA
CTTCAGAGTCACTAAGTCCGAACACATGACCCAATTGAAGGAAA
AGTTCAGAAAGATTTGTGATAAGTCTATGATCAGAAAAAGAAAC
TGTTTCTTGAACGAAGAACACTTGAAACAAAACCCTAGATTAGT
TGAACATGAAATGCAAACTTTAGATGCCAGACAAGATATGTTGG
TCGTCGAAGTCCCAAAGTTGGGTAAGGACGCTTGTGCCAAGGCT
ATCAAGGAATGGGGTCAACCAAAGTCTAAGATTACTCATTTGAT
CTTCACTTCCGCCTCTACCACCGATATGCCAGGTGCTGATTACC
ATTGTGCTAAGTTGTTGGGTTTATCCCCATCTGTTAAAAGAGTT
ATGATGTACCAATTGGGTTGTTATGGTGGTGGTACTGTTTTGAG
AATTGCCAAAGACATCGCTGAAAACAATAAGGGTGCTAGAGTTT
TGGCTGTTTGTTGTGATATTATGGCTTGTTTGTTCAGAGGTCCA
TCCGAGTCTGATTTAGAGTTGTTAGTTGGTCAAGCTATTTTCGG
TGACGGTGCTGCTGCTGTTATTGTTGGTGCTGAACCAGACGAAT
CTGTTGGTGAACGTCCAATCTTTGAATTGGTCTCTACCGGTCAA
ACCATCTTGCCAAACTCTGAAGGTACCATTGGTGGTCACATCAG
AGAAGCTGGTTTGATCTTCGATTTGCATAAAGATGTTCCTATGT
TGATTTCTAATAACATCGAAAAGTGCTTAATCGAAGCTTTCACT
CCAATCGGTATCTCTGATTGGAATTCCATTTTCTGGATTACCCA
TCCAGGTGGTAAGGCCATCTTGGATAAGGTTGAAGAAAAGTTGC
ATTTAAAGTCTGATAAGTTCGTTGACTCTCGTCACGTTTTGTCT
GAACATGGTAACATGTCTTCTTCCACTGTTTTGTTTGTTATGGA
TGAATTGAGAAAAAGATCCTTGGAAGAAGGTAAGTCTACTACTG
GTGATGGTTTTGAATGGGGTGTCTTGTTCGGTTTTGGTCCAGGT
TTGACCGTTGAAAGAGTTGTCGTTAGATCCGTTCCAATCAAGTA
CTAAGTATACTTCTTTTTTTTACTTTGTTCAGAACAACTTCTCA
TTTTTTTCTACTCATAACTTTAGCATCACAAAGTACACAATAAT
AACGAGTAGTAACACTTTTATAGTTCATACATGCTTCAACTACT
TAATAAATGATTGTATGATAATGTTTTCAATGTAAGAGATTTCG
ATTATCCACAAACTTTGAAACACAGGGACACAATTCTTGATATG
CTTTCAACCGCTGCGTTTTGGATACCTATTCTTGACATAATATG
ACTACCATTTTGTTATTGTACGTGGGGCAGTTGACGTCTTATCA
TATGTCAAAGTCATTTGCGAAGTTCTTGGCAAGTTGCCAACTGA
CGAGATGCAGTAAAAAGAGATTGCCGTCTTGAAACTTTTTGTCC
TTTTTTTTTTCCGGGGACTCTACGAGAACCCTTTGTCCTACTGA
TCCCCGCGTGCTTGGCCGGCCGTGATCATCTACCCATGCCGAAA
TTCGGGCCGTTGGCCGGATTGCGCGTTGTCTTCTCCGGTATCGA
AATCGCCGGACCGTTTGCCGGGCAAATGTTCGCAGAATGGGGCG
CGGAAGTTATCTGGATCGAGAACGTCGCCTGGGCCGACACCATT
CGCGTTCAACCGAACTACCCGCAACTCTCCCGCCGCAATTTGCA
CGCGCTGTCGTTAAATATTTTCAAAGATGAAGGCCGCGAAGCGT
TTCTGAAATTAATGGAAACCACCGATATCTTCATCGAAGCCAGT
AAAGGTCCGGCCTTTGCCCGTCGTGGCATTACCGATGAAGTACT
GTGGCAGCACAACCCGAAACTGGTTATCGCTCACCTGTCCGGTT
TTGGTCAGTACGGCACCGAGGAGTACACCAATCTTCCGGCCTAT
AACACTATCGCCCAGGCCTTTAGTGGTTACCTGATTCAGAACGG
TGATGTTGACCAGCCAATGCCTGCCTTCCCGTATACCGCCGATT
ACTTTTCTGGCCTGACCGCCACCACGGCGGCGCTGGCAGCACTG
CATAAAGTGCGTGAAACCGGTAAAGGCGAAAGTATCGACATCGC
CATGTATGAAGTGATGCTGCGTATGGGCCAGTACTTCATGATGG
ATTACTTCAACGGCGGCGAAATGTGCCCGCGCATGAGCAAAGGT
AAAGATCCCTACTACGCCGACGGCCGGCCAAGCACGCGGGGATC
AGTAGGACAAAGGGTTCTCGTAGAGTCCCCGGAAAAAAAAAAGG
ACAAAAAGTTTCAAGACGGCAATCTCTTTTTACTGCATCTCGTC
AGTTGGCAACTTGCCAAGAACTTCGCAAATGACTTTGACATATG
ATAAGACGTCAACTGCCCCACGTACAATAACAAAATGGTAGTCA
TATTATGTCAAGAATAGGTATCCAAAACGCAGCGGTTGAAAGCA
TATCAAGAATTGTGTCCCTGTGTTTCAAAGTTTGTGGATAATCG
AAATCTCTTACATTGAAAACATTATCATACAATCATTTATTAAG
TAGTTGAAGCATGTATGAACTATAAAAGTGTTACTACTCGTTAT
TATTGTGTACTTTGTGATGCTAAAGTTATGAGTAGAAAAAAATG
AGAAGTTGTTCTGIACAAAGTAAAAAAAAGAAGTATACTTACTT
TCTAGGGGTGTAATCAAAGATCAACAACTTTTCCCAGAAAGATC
TGTAAACGTCACCGAAACCAACATGAGCTGGGTGAATAATGTAG
TCTTGGATAGTTTCAACAGATTCGAAGGTGACTTCAACAATATG
AGTGTAACCTTCTTCTTTGTTCTTTTGGGTGACGTCTTTACCCC
AGTAGACATCCTTCATAGCTGGAATAATGTTAACCAAGTTAACG
TAAGTTTTGAAGAATTCCTCTTTTTGGGCTTCGGTAATTTCGTC
TTTGAACTTTAAGACGATCAAGTGTTTAACAGCCATTATAGTTT
TTTCTCCTTGACGTTAAAGTATAGAGGTATATTAACAATTTTTT
GTTGATACTTTTATGACATTTGAATAAGAAGTAATACAAACCGA
AAATGTTGAAAGTATTAGTTAAAGTGGTTATGCAGCTTTTGCAT
TTATATATCTGTTAATAGATCAAAAATCATCGCTTCGCTGATTA
ATTACCCCAGAAATAAGGCTAAAAAACTAATCGCATTATTATCC
TATGGTTGTTAATTTGATTCGTTGATTTGAAGGTTTGTGGGGCC
AGGTTACTGCCAATTTTTCCTCTTCATAACCATAAAAGCTAGTA
TTGTAGAATCTTTATTGTTCGGAGCAGTGCGGCGCGAGGCACAT
CTGCGTTTCAGGAACGCGACCGGTGAAGACCAGGACGCACGGAG
GAGAGTCTTCCGTCGGAGGGCTGTCGCCCGCTCGGCGGCTTCTA
ATCCGTACTTCAATATAGCAATGAGCAGTTAAGCGTATTACTGA
AAGTTCCAAAGAGAAGGTTTTTTTAGGCTAAGATAATGGGGCTC
TTTACATTTCCACAACATATAAGTAAGATTAGATATGGATATGT
ATATGGTGGTATTGCCATGTAATATGATTATTAAACTTCTTTGC
GTCCATCCAAAAAAAAAGTAAGAATTTTTGAAAATTCAATATAA
ATGGCTGTTAAACACTTGATCGTCTTAAAGTTCAAAGACGAAAT
TACCGAAGCCCAAAAAGAGGAATTCTTCAAAACTTACGTTAACT
TGGTTAACATTATTCCAGCTATGAAGGATGTCTACTGGGGTAAA
GACGTCACCCAAAAGAACAAAGAAGAAGGTTACACTCATATTGT
TGAAGTCACCTTCGAATCTGTTGAAACTATCCAAGACTACATTA
TTCACCCAGCTCATGTTGGTTTCGGTGACGTTTACAGATCTTTC
TGGGAAAAGTTGTTGATCTTTGATTACACCCCTAGAAAGTAATT
TGCCAGCTTACTATCCTTCTTGAAAATATGCACTCTATATCTTT
TAGTTCTTAATTGCAACACATAGATTTGCTGTATAACGAATTTT
ATGCTATTTTTTAAATTTGGAGTTCAGTGATAAAAGTGTCACAG
CGAATTTCCTCACATGTAGGGACCGAATTGTTTACAAGTTCTCT
GTACCACCATGGAGACATCAAAGATTGAAAATCTATGGAAAGAT
ATGGACGGTAGCAACAAGAATATAGCACGAGCCGCGAAGTTCAT
TTCGTTACTTTTGATATCGCTCACAACTATTGCGAAGCGCTTCA
GTGAAAAAATCATAAGGAAAAGTTGTAAATATTATTGGTAGTAT
TCGTTTGGTAAAGTAGAGGGGGTAATTTTTCCCCTTTATTTTGT
TCATACATTCTTAAATTGCTTTGCCTCTCCTTTTGGAAAGCTAG
GTCCGCCGGCGTTGGACGAGCGACTTTAATGTCGTTCTCCCTTT
TTAAAGAGTAAATACATATTTAAAAAAGTGACTATGGCTATTGC
TAAACGTGATAAAAATCAGAGCCTATAACACTCTCTGAAATAAC
GCTATGCAGGAATTTCCAGTTAAGTTCTTCTTGGGGTGACTTCT
TTACTCGGTATGATATGTGTTTTATATGCACAGTACGAGTCCAT
TAGGGTAAATTAGTGGCCGAGAAACTTTTGCCGCCGAGCTTTTA
AGTATCCTTTTGCCACTTCTTATTTAGATAAAGACCTGGCAGTA
GTAGTCGTAGAAGATAAGATAGACAGAGAATGAATACTAATAAG
ATAGCACAAGACGAAGTCCAAGATAAGGTTTTGCAAAGAGCAGA
ACTAGCACATTCTGTATGGAACTTAAGGTTCAACCTCAGTAAAG
TTGCCAAACGGATTCGCATGGAAACAAAGGTATTTCCAGAGATA
AAGATAAATGACGCGCAATCACAGTTAGAGCGATCTAGGTGTAG
AATATTTAGCCCTGACCTGGAGGAAGAACATGTGCCCTTGATTC
AAGGCGGCGGTTTAAACGCGTGGCCGTGCCGTC
MS101229 (SEQ ID NO: 6):
GACGGCACGGCCACGCGTTTAAACCGCCTACGCCATCATTAAAG
ACCTGGTCAACTATAAAATAATACAATCAATACTTGCTTGAACG
CTTGATTTTACTGATATTCTATCCAAAAGCAAGTAGACCAGAAA
CTCTCAAGATGTTGCAAATACCGTTCGATGTTTTTGGTTTAGAT
TGTTTTAATGTTGATGCTTTTTTACTTATTTTTGGAAGCGTCTT
TTTAATTTAGTTTTATATTATAGGTATATGAATGTGTTTATGCC
AATAAGGGTTTTTTTGTACAGTTATGTGATTATAAACAGTCTTT
TGTCTAGTTTTTTTCACCAGTATCGGCCTCTATTTATAAAAAAC
GGAGCAGCTTTCGGTGTCAGTAATTCTGAAAAAATTTGTGTCAC
TCTGATTGTAAATGAATTAATTTAGCTAGATAGTTGCGAGCCCC
AACGAGAAGATTGTCAGACAAAGACAACATTCAACAACCTACAT
CCGTTACTATTCGTTAACTCGAGGTACTTGAAACTTTTCAGTTA
AGTCGCTCGTCCAACGCCGGCGGACCTAGCTTTCCAAAAGGAGA
GGCAAAGCAATTTAAGAATGTATGAACAAAATAAAGGGGAAAAA
TTACCCCCTCTACTTTACCAAACGAATACTACCAATAATATTTA
CAACTTTTCCTTATGATTTTTTCACTGAAGCGCTTCGCAATAGT
TGTGAGCGATATCAAAAGTAACGAAATGAACTTCGCGGCTCGTG
CTATATTCTTGTTGCTACCGTCCATATCTTTCCATAGATTTTCA
ATCTTTGATGTCTCCATGGTGGTACAGAGAACTTGTAAACAATT
CGGTCCCTACATGTGAGGAAATTCGCTGTGACACTTTTATCACT
GAACTCCAAATTTAAAAAATAGCATAAAATTCGTTATACAGCAA
ATCTATGTGTTGCAATTAAGAACTAAAAGATATAGAGTGCATAT
TTTCAAGAAGGATAGTAAGCTGGCAAATTACTTTCTAGGGGTGT
AATCAAAGATCAACAACTTTTCCCAGAAAGATCTGTAAACGTCA
CCGAAACCAACATGAGCTGGGTGAATAATGTAGTCTTGGATAGT
TTCAACAGATTCGAAGGTGACTTCAACAATATGAGTGTAACCTT
CTTCTTTGTTCTTTTGGGTGACGTCTTTACCCCAGTAGACATCC
TTCATAGCTGGAATAATGTTAACCAAGTTAACGTAAGTTTTGAA
GAATTCCTCTTTTTGGGCTTCGGTAATTTCGTCTTTGAACTTTA
AGACGATCAAGTGTTTAACAGCCATTTATATTGAATTTTCAAAA
ATTCTTACTTTTTTTTTGGATGGACGCAAAGAAGTTTAATAATC
ATATTACATGGCAATACCACCATATACATATCCATATCTAATCT
TACTTATATGTTGTGGAAATGTAAAGAGCCCCATTATCTTAGCC
TAAAAAAACCTTCTCTTTGGAACTTTCAGTAATACGCTTAACTG
CTCATTGCTATATTGAAGTACGGATTAGAAGCCGCCGAGCGGGC
GACAGCCCTCCGACGGAAGACTCTCCTCCGTGCGTCCTGGTCTT
CACCGGTCGCGTTCCTGAAACGCAGATGTGCCTCGCGCCGCACT
GCTCCGAACAATAAAGATTCTACAATACTAGCTTTTATGGTTAT
GAAGAGGAAAAATTGGCAGTAACCTGGCCCCACAAACCTTCAAA
TCAACGAATCAAATTAACAACCATAGGATAATAATGCGATTAGT
TTTTTAGCCTTATTTCTGGGGTAATTAATCAGCGAAGCGATGAT
TTTTGATCTATTAACAGATATATAAATGCAAAAGCTGCATAACC
ACTTTAACTAATACTTTCAACATTTTCGGTTTGTATTACTTCTT
ATTCAAATGTCATAAAAGTATCAACAAAAAATTGTTAATATACC
TCTATACTTTAACGTCAAGGAGAAAAAACTATAATGGCTGTTAA
ACACTTGATCGTCTTAAAGTTCAAAGACGAAATTACCGAAGCCC
AAAAAGAGGAATTCTTCAAAACTTACGTTAACTTGGTTAACATT
ATTCCAGCTATGAAGGATGTCTACTGGGGTAAAGACGTCACCCA
AAAGAACAAAGAAGAAGGTTACACTCATATTGTTGAAGTCACCT
TCGAATCTGTTGAAACTATCCAAGACTACATTATTCACCCAGCT
CATGTTGGTTTCGGTGACGTTTACAGATCTTTCTGGGAAAAGTT
GTTGATCTTTGATTACACCCCTAGAAAGTAAGTATACTTCTTTT
TTTTACTTTGTTCAGAACAACTTCTCATTTTTTTCTACTCATAA
CTTTAGCATCACAAAGTACACAATAATAACGAGTAGTAACACTT
TTATAGTTCATACATGCTTCAACTACTTAATAAATGATTGTATG
ATAATGTTTTCAATGTAAGAGATTTCGATTATCCACAAACTTTG
AAACACAGGGACACAATTCTTGATATGCTTTCAACCGCTGCGTT
TTGGATACCTATTCTTGACATAATATGACTACCATTTTGTTATT
GTACGTGGGGCAGTTGACGTCTTATCATATGTCAAAGTCATTTG
CGAAGTTCTTGGCAAGTTGCCAACTGACGAGATGCAGTAAAAAG
AGATTGCCGTCTTGAAACTTTTTGTCCTTTTTTTTTTCCGGGGA
CTCTACGAGAACCCTTTGTCCTACTGATCCCCGCGTGCTTGGCC
GGCCGTGCGAGTAAGCAACTCTGGCGCTGGCATGGCATAACCGG
CGACGGCAATGCGCAAGATGGGATGCTATGGGCAGAGAGCCGTA
CTTTACTGCTTATGGCACTACAGCAACAGATGGTTACCCCACTA
AGCCTGAAGCGAATCGCCATCAATTCTGCGCAGTGGCGAGGAGA
TAAAAGCGCGGAAGTCATTCATCAACTGGCGACGCTACTCAAAG
CAGGGTTAACGCTTTCTGAAGGGCTGGCTCTGCTGGCGGAACAG
CATCCCAGTAAGCAATGGCAAGCGTTGCTGCAATCGCTGGCGCA
CGATCTCGAACAGGGCATTGCTTTTTCCAATGCCTTATTACCCT
GGTCAGAGGTATTTCCGCCGCTCTATCAGGCGATGATCCGCACG
GGTGAACTGACCGGTAAGCTGGATGAATGCTGCTTTGAACTGGC
GCGTCAGCAAAAAGCCCAGCGTCAGTTGACCGACAAAGTGAAAT
CAGCGTTACGTTATCCCATCATCATTTTAGCGATGGCAATCATG
GTGGTTGTGGCAATGCTGCATTTTGTTCTGCCGGAGTTTGCCGC
TATCTATAAGACCTTCAACACCCCACTACCGGCACTAACGCAGG
GGATCATGACGCTGGCAGACTTTAGTGGCGAATGGAGCTGGCTG
CTGGTGTTGTTCGGCTTTCTGCTGGCGATAGCCAATAAGTTGCT
GAACGGCCGGCCAAGCACGCGGGGATCAGTAGGACAAAGGGTTC
TCGTAGAGTCCCCGGAAAAAAAAAAGGACAAAAAGTTTCAAGAC
GGCAATCTCTTTTTACTGCATCTCGTCAGTTGGCAACTTGCCAA
GAACTTCGCAAATGACTTTGACATATGATAAGACGTCAACTGCC
CCACGTACAATAACAAAATGGTAGTCATATTATGTCAAGAATAG
GTATCCAAAACGCAGCGGTTGAAAGCATATCAAGAATTGTGTCC
CTGTGTTTCAAAGTTTGTGGATAATCGAAATCTCTTACATTGAA
AACATTATCATACAATCATTTATTAAGTAGTTGAAGCATGTATG
AACTATAAAAGTGTTACTACTCGTTATTATTGTGTACTTTGTGA
TGCTAAAGTTATGAGTAGAAAAAAATGAGAAGTTGTTCTGAACA
AAGTAAAAAAAAGAAGTATACTTATTCAAAATGGGAGAATTGTT
GACGCAAAACTCTACGCATGATCTTGTTGGTGGCAGTTCTAGGC
AAAGAAGACAAAGGGACGACTCTAGTAACCTTAAACAATGGATT
CAACTTCTTTTGCAAACCCAAGTTGAAGGACAATCTCAATTGGT
TCAAGTCGATAGTAGTATCGTTAGAATCCTTCAAGACGAAGAAA
ATAACCAATTGTTCTGGACCACCACCTAATGGTGGAACACCGAT
AGCAGTGGTTTCGAAAACTCTGTCATCGACTTCGTTACAAACTC
TTTCAATCTCAATGGAAGAGATTTTGATACCACCGATGTTCATG
GTGTCATCAGCACGACCGTGAGCATGGTAGTAACCGTTGGAAGT
TAATTCAAAGATGTCACCGTGTCTTCTCAAAACTTCACCGTTCA
AAGTTGGCATACCTTTGAAGTAGACATCGTGGTGGTTACCGTTC
AATAAAGTCTTAGAAGCACCGAACATAACTGGACCCAAAGCCAA
TTCACCAATACCTGGCTTGTTCTTTGGCATTGGGTAACCGTTCT
TATCCAAAATGTACAAAGTACAACCCATACATTGGGAAGAAAAG
GAGGACAAGGATTGGGCTTGTAAGAAAGAACCAGCAGAGAAAGC
ACCACCGATTTCGGTACCACCACACATTTCGATAACAGGTTTAT
AGTTGGCTCTACCCATCAACCACAAGTATTCATCGACGTTAGAA
GCTTCACCAGAGGAAGAAAAGCAACGGATGGTAGACCAGTCATA
ACCGGAAACGCAGTTGGTGGATTTCCAAGATCTAACAATAGATG
GAACAACACCTAACATAGTAACCTTAGCGTCTTGGACGAACTTG
GCGAAACCAGAAACCAATGGGGAACCATTATACAAAGCGATAGA
AGCACCGTTCAATAAAGAGGCGTAAACCAACCATGGACCCATCA
TCCAACCTAAATTAGTTGGCCAAACAATGACGTCACCTTTACGA
ATATCCAAGTGAGACCAACCGTCGGCAGCAGCCTTCAATGGAGT
AGCTTGGGTCCATGGAATGGCCTTTGGTTCACCAGTGGTACCGG
AAGAGAATAAAATGTTGGTGTAGGCATCAACTGGTTGTTCACGA
GCGGTGAATTCACAGTTCTTGAATTCCTTAGCACGTTCCAAGAA
ATAATCCCAGGAAATGTCACCGTCACGCAATTCGGCACCGATGT
TGGAACCGGAACATGGAATGACAATAGCCATTGGAGACTTAGCT
TCAACGACTCTAGAATACAATGGAATTCTCTTCTTACCACGGAT
GATGTGGTCTTGAGTGAAGATGGCCTTAGCCTTAGACAATCTCA
ATCTAGTAGAGATTTCTGGAGCGGAGAAAGAATCAGCGATGGAA
ACGACGACGTAACCAGCCAAGACAATGGCTAAGTAGATGACGAC
AGCGTCAACGTGCATTGGCATATCGATGGCGATAGCACAACCTT
TTTCCAAACCCATTTCTTCCAAGGCATAACCAACCAACCAAACT
CTCTTTCTCAATTGGTCCAAAGTCAACTTGTTCAATGGCAAATC
GTCGTTACCCTCATCACGCCAAACGATCATAGTATCATTCAACT
TTTTGTTAGAGTTAACATTCAAGCAGTTCTTAGCAGAGTTCAAG
TAACCACCTGGCAACCATTCGGAACCACCTGGGTTGTTAATATC
GTCTCTACGTAAGATACATTCTGGATCTTTAGAAAAGGAGATCT
TCATTTCATCCATTAAAACAGTTCTCCAGTAAACTTCTGGGTTT
CTGACGGAGAACTCTTGGAAGTGAGAAAAAGAAGAAATTGGATC
CTTGTATTTAACACCCAAGAATTCCTTACCTCTTTTCTCCAACA
AAGCACCCAAGTTGGTAGACTTGACCTTTTCAGGGTCTGGAATC
CAAGCTGGTGGGGCTGGACCAAAGTCCTTGTAACAACCATAGAA
TAACATTTGGTGCAAGGAAAATGGCAAGTCTGGGGATAAGATAT
GGTTGGCAATGTTAATCCAAGTTTGTGGGGTAGCAGCACCGTAA
TTACAAACAATTTCAGCTAATCTACCATGCAAAGTTTCGGCGAC
CTCAGAGGTAATACCCAAAGCGATGAAATCGGAAGCAACAACAG
AATCCAAAGATTTGTAGTTCTTACCCATTATAGTTTTTTCTCCT
TGACGTTAAAGTATAGAGGTATATTAACAATTTTTTGTTGATAC
TTTTATGACATTTGIATAAGAAGTAATACAAACCGAAAATGTTG
AAAGTATTAGTTAAAGTGGTTATGCAGCTTTTGCATTTATATAT
CTGTTAATAGATCAAAAATCATCGCTTCGCTGATTAATTACCCC
AGAAATAAGGCTAAAAAACTAATCGCATTATTATCCTATGGTTG
TTAATTTGATTCGTTGATTTGAAGGTTTGTGGGGCCAGGTTACT
GCCAATTTTTCCTCTTCATAACCATAAAAGCTAGTATTGTAGAA
TCTTTATTGTTCGGAGCAGTGCGGCGCGAGGCACATCTGCGTTT
CAGGAACGCGACCGGTGAAGACCAGGACGCACGGAGGAGAGTCT
TCCGTCGGAGGGCTGTCGCCCGCTCGGCGGCTTCTAATCCGTAC
TTCAATATAGCAATGAGCAGTTAAGCGTATTACTGAAAGTTCCA
AAGAGAAGGTTTTTTTAGGCTAAGATAATGGGGCTCTTTACATT
TCCACAACATATAAGTAAGATTAGATATGGATATGTATATGGTG
GTATTGCCATGTAATATGATTATTAAACTTCTTTGCGTCCATCC
AAAAAAAAAGTAAGAATTTTTGAAAATTCAATATAAATGAACCA
CTTAAGAGCTGAAGGTCCAGCTTCCGTTTTGGCCATTGGTACCG
CTAACCCAGAAAACATCTTGTTGCAAGACGAATTTCCAGACTAC
TACTTCAGAGTCACTAAGTCCGAACACATGACCCAATTGAAGGA
AAAGTTCAGAAAGATTTGTGATAAGTCTATGATCAGAAAAAGAA
ACTGTTTCTTGAACGAAGAACACTTGAAACAAAACCCTAGATTA
GTTGAACATGAAATGCAAACTTTAGATGCCAGACAAGATATGTT
GGTCGTCGAAGTCCCAAAGTTGGGTAAGGACGCTTGTGCCAAGG
CTATCAAGGAATGGGGTCAACCAAAGTCTAAGATTACTCATTTG
ATCTTCACTTCCGCCTCTACCACCGATATGCCAGGTGCTGATTA
CCATTGTGCTAAGTTGTTGGGTTTATCCCCATCTGTTAAAAGAG
TTATGATGTACCAATTGGGTTGTTATGGTGGTGGTACTGTTTTG
AGAATTGCCAAAGACATCGCTGAAAACAATAAGGGTGCTAGAGT
TTTGGCTGTTTGTTGTGATATTATGGCTTGTTTGTTCAGAGGTC
CATCCGAGTCTGATTTAGAGTTGTTAGTTGGTCAAGCTATTTTC
GGTGACGGTGCTGCTGCTGTTATTGTTGGTGCTGAACCAGACGA
ATCTGTTGGTGAACGTCCAATCTTTGAATTGGTCTCTACCGGTC
AAACCATCTTGCCAAACTCTGAAGGTACCATTGGTGGTCACATC
AGAGAAGCTGGTTTGATCTTCGATTTGCATAAAGATGTTCCTAT
GTTGATTTCTAATAACATCGAAAAGTGCTTAATCGAAGCTTTCA
CTCCAATCGGTATCTCTGATTGGAATTCCATTTTCTGGATTACC
CATCCAGGTGGTAAGGCCATCTTGGATAAGGTTGAAGAAAAGTT
GCATTTAAAGTCTGATAAGTTCGTTGACTCTCGTCACGTTTTGT
CTGAACATGGTAACATGTCTTCTTCCACTGTTTTGTTTGTTATG
GATGAATTGAGAAAAAGATCCTTGGAAGAAGGTAAGTCTACTAC
TGGTGATGGTTTTGAATGGGGTGTCTTGTTCGGTTTTGGTCCAG
GTTTGACCGTTGAAAGAGTTGTCGTTAGATCCGTTCCAATCAAG
TACTAATTTGCCAGCTTACTATCCTTCTTGAAAATATGCACTCT
ATATCTTTTAGTTCTTAATTGCAACACATAGATTTGCTGTATAA
CGAATTTTATGCTATTTTTTAAATTTGGAGTTCAGTGATAAAAG
TGTCACAGCGAATTTCCTCACATGTAGGGACCGAATTGTTTACA
AGTTCTCTGTACCACCATGGAGACATCAAAGATTGAAAATCTAT
GGAAAGATATGGACGGTAGCAACAAGAATATAGCACGAGCCGCG
AAGTTCATTTCGTTACTTTTGATATCGCTCACAACTATTGCGAA
GCGCTTCAGTGAAAAAATCATAAGGAAAAGTTGTAAATATTATT
GGTAGTATTCGTTTGGTAAAGTAGAGGGGGTAATTTTTCCCCTT
TATTTTGTTCATACATTCTTAAATTGCTTTGCCTCTCCTTTTGG
AAAGCTAGGTCCGCCGGCGTTGGACGAGCGAAAATTCATTTAAT
ATTCAATGAAGTTATAAATTGATAGTTTAAATAAAGTCAGTCTT
TTTCCTCATGTTTAGAATTGTATTAATGTACGCCGTTTACGTTG
GAGTGTAAATGTGTCTATTCCAGAACGAAATCTAAATGAGCAGT
ACAGGCAGTTCAGATGGTACTGAAGCGGTACTAAAGATGCATGA
ATTGAACAGATGTGGTAGTTATGTATATGAGGAATATGAGTTGT
CACATTAAAAATATAATAGCTATGATCCCATTATTATATTCGTG
ACAGTTCGTAACGTTTTAATTGGCTTATGTTTTTGAGAAATGGG
TGAATTTTAAGATAATTGTTGGGATTCCATTATTGATAAAGGCT
ATAATATTAGGTATACAGAATATACTGGAAGTTCTCCTCGAGGA
TATAGGAATCCTCAAAATGGAATCTATATTTCTATTTACTAATA
TCACGATTATTCTTCATTCCGTTTTATATGTTTCATTATCCTAT
TACATTATCAATCCTTGCATTTCAGCTTCCTCTAACTTCGATGA
CAGCTGGCGGTTTAAACGCGTGGCCGTGCCGTC
Sequences of individual cannabinoid pathway
genes HCS> nucleic acid sequence
(SEQ ID NO: 7)
ATGGGTAAGAACTACAAATCTTTGGATTCTGTTGTTGCTTCCGA
TTTCATCGCTTTGGGTATTACCTCTGAGGTCGCCGAAACTTTGC
ATGGTAGATTAGCTGAAATTGTTTGTAATTACGGTGCTGCTACC
CCACAAACTTGGATTAACATTGCCAACCATATCTTATCCCCAGA
CTTGCCATTTTCCTTGCACCAAATGTTATTCTATGGTTGTTACA
AGGACTTTGGTCCAGCCCCACCAGCTTGGATTCCAGACCCTGAA
AAGGTCAAGTCTACCAACTTGGGTGCTTTGTTGGAGAAAAGAGG
TAAGGAATTCTTGGGTGTTAAATACAAGGATCCAATTTCTTCTT
TTTCTCACTTCCAAGAGTTCTCCGTCAGAAACCCAGAAGTTTAC
TGGAGAACTGTTTTAATGGATGAAATGAAGATCTCCTTTTCTAA
AGATCCAGAATGTATCTTACGTAGAGACGATATTAACAACCCAG
GTGGTTCCGAATGGTTGCCAGGTGGTTACTTGAACTCTGCTAAG
AACTGCTTGAATGTTAACTCTAACAAAAAGTTGAATGATACTAT
GATCGTTTGGCGTGATGAGGGTAACGACGATTTGCCATTGAACA
AGTTGACTTTGGACCAATTGAGAAAGAGAGTTTGGTTGGTTGGT
TATGCCTTGGAAGAAATGGGTTTGGAAAAAGGTTGTGCTATCGC
CATCGATATGCCAATGCACGTTGACGCTGTCGTCATCTACTTAG
CCATTGTCTTGGCTGGTTACGTCGTCGTTTCCATCGCTGATTCT
TTCTCCGCTCCAGAAATCTCTACTAGATTGAGATTGTCTAAGGC
TAAGGCCATCTTCACTCAAGACCACATCATCCGTGGTAAGAAGA
GAATTCCATTGTATTCTAGAGTCGTTGAAGCTAAGTCTCCAATG
GCTATTGTCATTCCATGTTCCGGTTCCAACATCGGTGCCGAATT
GCGTGACGGTGACATTTCCTGGGATTATTTCTTGGAACGTGCTA
AGGAATTCAAGAACTGTGAATTCACCGCTCGTGAACAACCAGTT
GATGCCTACACCAACATTTTATTCTCTTCCGGTACCACTGGTGA
ACCAAAGGCCATTCCATGGACCCAAGCTACTCCATTGAAGGCTG
CTGCCGACGGTTGGTCTCACTTGGATATTCGTAAAGGTGACGTC
ATTGTTTGGCCAACTAATTTAGGTTGGATGATGGGTCCATGGTT
GGTTTACGCCTCTTTATTGAACGGTGCTTCTATCGCTTTGTATA
ATGGTTCCCCATTGGTTTCTGGTTTCGCCAAGTTCGTCCAAGAC
GCTAAGGTTACTATGTTAGGTGTTGTTCCATCTATTGTTAGATC
TTGGAAATCCACCAACTGCGTTTCCGGTTATGACTGGTCTACCA
TCCGTTGCTTTTCTTCCTCTGGTGAAGCTTCTAACGTCGATGAA
TACTTGTGGTTGATGGGTAGAGCCAACTATAAACCTGTTATCGA
AATGTGTGGTGGTACCGAAATCGGTGGTGCTTTCTCTGCTGGTT
CTTTCTTACAAGCCCAATCCTTGTCCTCCTTTTCTTCCCAATGT
ATGGGTTGTACTTTGTACATTTTGGATAAGAACGGTTACCCAAT
GCCAAAGAACAAGCCAGGTATTGGTGAATTGGCTTTGGGTCCAG
TTATGTTCGGTGCTTCTAAGACTTTATTGAACGGTAACCACCAC
GATGTCTACTTCAAAGGTATGCCAACTTTGAACGGTGAAGTTTT
GAGAAGACACGGTGACATCTTTGAATTAACTTCCAACGGTTACT
ACCATGCTCACGGTCGTGCTGATGACACCATGAACATCGGTGGT
ATCAAAATCTCTTCCATTGAGATTGAAAGAGTTTGTAACGAAGT
CGATGACAGAGTTTTCGAAACCACTGCTATCGGTGTTCCACCAT
TAGGTGGTGGTCCAGAACAATTGGTTATTTTCTTCGTCTTGAAG
GATTCTAACGATACTACTATCGACTTGAACCAATTGAGATTGTC
CTTCAACTTGGGTTTGCAAAAGAAGTTGAATCCATTGTTTAAGG
TTACTAGAGTCGTCCCTTTGTCTTCTTTGCCTAGAACTGCCACC
AACAAGATCATGCGTAGAGTTTTGCGTCAACAATTCTCCCATTT
TGAATAA
TKS> nucleic acid sequence (SEQ ID NO: 8)
ATGAACCACTTAAGAGCTGAAGGTCCAGCTTCCGTTTTGGCCAT
TGGTACCGCTAACCCAGAAAACATCTTGTTGCAAGACGAATTTC
CAGACTACTACTTCAGAGTCACTAAGTCCGAACACATGACCCAA
TTGAAGGAAAAGTTCAGAAAGATTTGTGATAAGTCTATGATCAG
AAAAAGAAACTGTTTCTTGAACGAAGAACACTTGAAACAAAACC
CTAGATTAGTTGAACATGAAATGCAAACTTTAGATGCCAGACAA
GATATGTTGGTCGTCGAAGTCCCAAAGTTGGGTAAGGACGCTTG
TGCCAAGGCTATCAAGGAATGGGGTCAACCAAAGTCTAAGATTA
CTCATTTGATCTTCACTTCCGCCTCTACCACCGATATGCCAGGT
GCTGATTACCATTGTGCTAAGTTGTTGGGTTTATCCCCATCTGT
TAAAAGAGTTATGATGTACCAATTGGGTTGTTATGGTGGTGGTA
CTGTTTTGAGAATTGCCAAAGACATCGCTGAAAACAATAAGGGT
GCTAGAGTTTTGGCTGTTTGTTGTGATATTATGGCTTGTTTGTT
CAGAGGTCCATCCGAGTCTGATTTAGAGTTGTTAGTTGGTCAAG
CTATTTTCGGTGACGGTGCTGCTGCTGTTATTGTTGGTGCTGAA
CCAGACGAATCTGTTGGTGAACGTCCAATCTTTGAATTGGTCTC
TACCGGTCAAACCATCTTGCCAAACTCTGAAGGTACCATTGGTG
GTCACATCAGAGAAGCTGGTTTGATCTTCGATTTGCATAAAGAT
GTTCCTATGTTGATTTCTAATAACATCGAAAAGTGCTTAATCGA
AGCTTTCACTCCAATCGGTATCTCTGATTGGAATTCCATTTTCT
GGATTACCCATCCAGGTGGTAAGGCCATCTTGGATAAGGTTGAA
GAAAAGTTGCATTTAAAGTCTGATAAGTTCGTTGACTCTCGTCA
CGTTTTGTCTGAACATGGTAACATGTCTTCTTCCACTGTTTTGT
TTGTTATGGATGAATTGAGAAAAAGATCCTTGGAAGAAGGTAAG
TCTACTACTGGTGATGGTTTTGAATGGGGTGTCTTGTTCGGTTT
TGGTCCAGGTTTGACCGTTGAAAGAGTTGTCGTTAGATCCGTTC
CAATCAAGTACTAA
OAC> nucleic acid sequence (SEQ ID NO: 9)
ATGGCTGTTAAACACTTGATCGTCTTAAAGTTCAAAGACGAAAT
TACCGAAGCCCAAAAAGAGGAATTCTTCAAAACTTACGTTAACT
TGGTTAACATTATTCCAGCTATGAAGGATGTCTACTGGGGTAAA
GACGTCACCCAAAAGAACAAAGAAGAAGGTTACACTCATATTGT
TGAAGTCACCTTCGAATCTGTTGAAACTATCCAAGACTACATTA
TTCACCCAGCTCATGTTGGTTTCGGTGACGTTTACAGATCTTTC
TGGGAAAAGTTGTTGATCTTTGATTACACCCCTAGAAAGTAA
HCS amino acid sequence (SEQ ID NO: 10):
MGKNYKSLDSVVASDFIALGITSEVAETLHGRLAEIVCNYGAAT
PQTWINIANHILSPDLPFSLHQMLFYGCYKDFGPAPPAWIPDPE
KVKSTNLGALLEKRGKEFLGVKYKDPISSFSHFQEFSVRNPEVY
WRTVLMDEMKISFSKDPECILRRDDINNPGGSEWLPGGYLNSAK
NCLNVNSNKKLNDTMIVWRDEGNDDLPLNKLTLDQLRKRVWLVG
YALEEMGLEKGCAIAIDMPMHVDAVVIYLAIVLAGYVVVSIADS
FSAPEISTRLRLSKAKAIFTQDHIIRGKKRIPLYSRVVEAKSPM
AIVIPCSGSNIGAELRDGDISWDYFLERAKEFKNCEFTAREQPV
DAYTNILFSSGTTGEPKAIPWTQATPLKAAADGWSHLDIRKGDV
IVWPTNLGWMMGPWLVYASLLNGASIALYNGSPLVSGFAKFVQD
AKVTMLGVVPSIVRSWKSTNCVSGYDWSTIRCFSSSGEASNVDE
YLWLMGRANYKPVIEMCGGTEIGGAFSAGSFLQAQSLSSFSSQC
MGCTLYILDKNGYPMPKNKPGIGELALGPVMFGASKTLLNGNHH
DVYFKGMPTLNGEVLRRHGDIFELTSNGYYHAHGRADDTMNIGG
IKISSIEIERVCNEVDDRVFETTAIGVPPLGGGPEQLVIFFVLK
DSNDTTIDLNQLRLSFNLGLQKKLNPLFKVTRVVPLSSLPRTAT
NKIMRRVLRQFSHFE
TKS amino acid sequence (SEQ ID NO: 11):
MNHLRAEGPASVLAIGTANPENILLQDEFPDYYFRVIKSEHMTQ
LKEKFRKICDKSMIRKRNCFLNEEHLKQNPRLVEHEMQTLDARQ
DMLVVEVPKLGKDACAKAIKEWGQPKSKITHLIFTSASTTDMPG
ADYHCAKLLGLSPSVKRVMMYQLGCYGGGTVLRIAKDIAENNKG
ARVLAVCCDIMACLFRGPSESDLELLVGQAIFGDGAAAVIVGAE
PDESVGERPIFELVSTGQIILPNSEGTIGGHIREAGLIFDLHKD
VPMLISNNIEKCLIEAFTPIGISDWNSIFWITHPGGKAILDKVE
EKLHLKSDKFVDSRHVLSEHGNMSSSTVLFVMDELRKRSLEEGK
STTGDGFEWGVLFGFGPGLTVERVVVRSVPIKY
OAC amino acid sequence (SEQ ID NO: 12):
MAVKHLIVLKFKDEITEAQKEEFFKTYVNLVNIIPAMKDVYWGK
DVTQKNKEEGYTHIVEVTFESVETIQDYIIHPAHVGFGDVYRSF
WEKLLIFDYTPRK
pGAL1 (SEQ ID NO: 13):
TGGAACTTTCAGTAATACGCTTAACTGCTCATTGCTATATTGAA
GTACGGATTAGAAGCCGCCGAGCGGGCGACAGCCCTCCGACGGA
AGACTCTCCTCCGTGCGTCCTGGTCTTCACCGGTCGCGTTCCTG
AAACGCAGATGTGCCTCGCGCCGCACTGCTCCGAACAATAAAGA
TTCTACAATACTAGCTTTTATGGTTATGAAGAGGAAAAATTGGC
AGTAACCTGGCCCCACAAACCTTCAAATCAACGAATCAAATTAA
CAACCATAGGATAATAATGCGATTAGTTTTTTAGCCTTATTTCT
GGGGTAATTAATCAGCGAAGCGATGATTTTTGATCTATTAACAG
ATATATAAATGCAAAAGCTGCATAACCACTTTAACTAATACTTT
CAACATTTTCGGTTTGTATTACTTCTTATTCAAATGTCATAAAA
GTATCAACAAAAAATTGTTAATATACCTCTATACTTTAACGTCA
AGGAGAAAAAACTATA
pGAL10 (SEQ ID NO: 14):
CATCGCTTCGCTGATTAATTACCCCAGAAATAAGGCTAAAAAAC
TAATCGCATTATTATCCTATGGTTGTTAATTTGATTCGTTGATT
TGAAGGTTTGTGGGGCCAGGTTACTGCCAATTTTTCCTCTTCAT
AACCATAAAAGCTAGTATTGTAGAATCTTTATTGTTCGGAGCAG
TGCGGCGCGAGGCACATCTGCGTTTCAGGAACGCGACCGGTGAA
GACCAGGACGCACGGAGGAGAGTCTTCCGTCGGAGGGCTGTCGC
CCGCTCGGCGGCTTCTAATCCGTACTTCAATATAGCAATGAGCA
GTTAAGCGTATTACTGAAAGTTCCAAAGAGAAGGTTTTTTTAGG
CTAAGATAATGGGGCTCTTTACATTTCCACAACATATAAGTAAG
ATTAGATATGGATATGTATATGGTGGTATTGCCATGTAATATGA
TTATTAAACTTCTTTGCGTCCATCCAAAAAAAAAGTAAGAATTT
TTGAAAATTCAATATAA
pGAL2 (SEQ ID NO: 15):
GGCTTAAGTAGGTTGCAATTTCTTTTTCTATTAGTAGCTAAAAA
TGGGTCACGTGATCTATATTCGAAAGGGGCGGTTGCCTCAGGAA
GGCACCGGCGGTCTTTCGTCCGTGCGGAGATATCTGCGCCGTTC
AGGGGTCCATGTGCCTTGGACGATATTAAGGCAGAAGGCAGTAT
CGGGGCGGATCACTCCGAACCGAGATTAGTTAAGCCCTTCCCAT
CTCAAGATGGGGAGCAAATGGCATTATACTCCTGCTAGAAAGTT
AACTGTGCACATATTCTTAAATTATACAATGTTCTGGAGAGCTA
TTGTTTAAAAAACAAACATTTCGCAGGCTAAAATGTGGAGATAG
GATTAGTTTTGTAGACATATATAAACAATCAGTAATTGGATTGA
AAATTTGGTGTTGTGAATTGCTCTTCATTATGCACCTTATTCAA
TTATCATCAAGAATAGCAATAGTTAAGTAAACACAAGATTAACA
TAATAAAAAAAATAATTCTTTCATA
pGAL3 (SEQ ID NO: 16):
TTTTACTATTATCTTCTACGCTGACAGTAATATCAAACAGTGAC
ACATATTAAACACAGTGGTTTCTTTGCATAAACACCATCAGCCT
CAAGTCGTCAAGTAAAGATTTCGTGTTCATGCAGATAGATAACA
ATCTATATGTTGATAATTAGCGTTGCCTCATCAATGCGAGATCC
GTTTAACCGGACCCTAGTGCACTTACCCCACGTTCGGTCCACTG
TGTGCCGAACATGCTCCTTCACTATTTTAACATGTGGAATTCTT
GAAAGAATGAAATCGCCATGCCAAGCCATCACACGGTCTTTTAT
GCAATTGATTGACCGCCTGCAACACATAGGCAGTAAAATTTTTA
CTGAAACGTATATAATCATCATAAGCGACAAGTGAGGCAACACC
TTTGTTACCACATTGACAACCCCAGGTATTCATACTTCCTATTA
GCGGAATCAGGAGTGCAAAAAGAGAAAATAAAAGTAAAAAGGTA
GGGCAACACATAGT
pGAL7 (SEQ ID NO: 17):
GGACGGTAGCAACAAGAATATAGCACGAGCCGCGAAGTTCATTT
CGTTACTTTTGATATCGCTCACAACTATTGCGAAGCGCTTCAGT
GAAAAAATCATAAGGAAAAGTTGTAAATATTATTGGTAGTATTC
GTTTGGTAAAGTAGAGGGGGTAATTTTTCCCCTTTATTTTGTTC
ATACATTCTTAAATTGCTTTGCCTCTCCTTTTGGAAAGCTATAC
TTCGGAGCACTGTTGAGCGAAGGCTCATTAGATATATTTTCTGT
CATTTTCCTTAACCCAAAAATAAGGGAAAGGGTCCAAAAAGCGC
TCGGACAACTGTTGACCGTGATCCGAAGGACTGGCTATACAGTG
TTCACAAAATAGCCAAGCTGAAAATAATGTGTAGCTATGTTCAG
TTAGTTTGGCTAGCAAAGATATAAAAGCAGGTCGGAAATATTTA
TGGGCATTATTATGCAGAGCATCAACATGATAAAAAAAAACAGT
TGAATATTCCCTCAAAA
pGAL4 (SEQ ID NO: 18):
GCGACACAGAGATGACAGACGGTGGCGCAGGATCCGGTTTAAAC
GAGGATCCCTTAAGTTTAAACAACAACAGCAAGCAGGTGTGCAA
GACACTAGAGACTCCTAACATGATGTATGCCAATAAAACACAAG
AGATAAACAACATTGCATGGAGGCCCCAGAGGGGCGATTGGTTT
GGGTGCGTGAGCGGCAAGAAGTTTCAAAACGTCCGCGTCCTTTG
AGACAGCATTCGCCCAGTATTTTTTTTATTCTACAAACCTTCTA
TAATTTCAAAGTATTTACATAATTCTGTATCAGTTTAATCACCA
TAATATCGTTTTCTTTGTTTAGTGCAATTAATTTTTCCTATTGT
TACTTCGGGCCTTTTTCTGTTTTATGAGCTATTTTTTCCGTCAT
CCTTCCCCAGATTTTCAGCTTCATCTCCAGATTGTGTCTACGTA
ATGCACGCCATCATTTTAAGAGAGGACAGAGAAGCAAGCCTCCT
GAAAG
pMAL1 (SEQ ID NO: 19):
GATGATGGAC ACTAGTGTGT CGAGAATGTA TCAACTATAT
ATAGTCCTAA TGCCACACAA ATATGAAGTG GGGGAAGCCC
ATTCTTAATC CGGCTCAATT TTGGTGCGTG ATCGCGGCCT
ATGTTTGCTT CCAGAAAAAG CTTAGAATAA TATTTCTCAC
CTTTGATGGA ATGCTCGCGA GTGCTCGTTT TGATTACCCC
ATATGCATTG TTGCAGCATG CAAGCACTAT TGCAAGCCAC
GCATGGAAGA AATTTGCAAA CACCTATAGC CCCGCGTTGT
TGAGGAGGTG GACTTGGTGT AGGACCATAA AGCTGTGCAC
TACTATGGTG AGCTCTGTCG TCTGGTGACC TTCTATCTCA
GGCACATCCT CGTTTTTGTG CATGAGGTTC GAGTCACGCC
CACGGCCTAT TAATCCGCGA AATAAATGCG AAATCTAAAT
TATGACGCAA GGCTGAGAGA TTCTGACACG CCGCATTTGC
GGGGCAGTAA TTATCGGGCA GTTTTCCGGG GTTCGGGATG
GGGTTTGGAG AGAAAGTTCA ACACAGACCA AAACAGCTTG
GGACCACTTG GATGGAGGTC CCCGCAGAAG AGCTCTGGCG
CGTTGGACAA ACATTGACAA TCCACGGCAA AATTGTCTAC
AGTTCCGTGT ATGCGGATAG GGATATCTTC GGGAGTATCG
CAATAGGATA CAGGCACTGT GCAGATTACG CGACATGATA
GCTTTGTATG TTCTACAGAC TCTGCCGTAG CAGTCTAGAT
ATAATATCGG AGTTTTGTAGCGTCGTAAGG AAAACTTGGG
TTACACAGGT TTCTTGAGAG CCCTTTGACG TTGATTGCTC
TGGCTTCCAT CCAGGCCCTC ATGTGGTTCA GGTGCCTCCG
CAGTGGCTGG CAAGCGTGGGGGTCAATTAC GTCACTTCTA
TTCATGTACC CCAGACTCAA TTGTTGACAG CAATTTCAGC
GAGAATTAAA TTCCACAATC AATTCTCGCT GAAATAATTA
GGCCGTGATT TAATTCTCGCTGAAACAGAA TCCTGTCTGG
GGTACAGATA ACAATCAAGT AACTATTATG GACGTGCATA
GGAGGTGGAG TCCATGACGC AAAGGGAAAT ATTCATTTTA
TCCTCGCGAA GTTGGGATGTGTCAAAGCGT CGCGCTCGCT
ATAGTGATGA GAATGTCTTT AGTAAGCTTA AGCCATATAA
AGACCTTCCG CCTCCATATT TTTTTTTATC CCTCTTGACA
ATATTAATTC CTT
pMAL2 (SEQ ID NO: 20):
AAGGAATTAA TATTGTCAAG AGGGATAAAA AAAAATATGG
AGGCGGAAGG TCTTTATATG GCTTAAGCTT ACTAAAGACA
TTCTCATCAC TATAGCGAGC GCGACGCTTT GACACATCCC
AACTTCGCGA GGATAAAATG AATATTTCCC TTTGCGTCAT
GGACTCCACC TCCTATGCACGTCCATAATA GTTACTTGAT
TGTTATCTGT ACCCCAGACA GGATTCTGTT TCAGCGAGAA
TTAAATCACG GCCTAATTAT TTCAGCGAGA ATTGATTGTG
GAATTTAATT CTCGCTGAAATTGCTGTCAA CAATTGAGTC
TGGGGTACAT GAATAGAAGT GACGTAATTG ACCCCCACGC
TTGCCAGCCA CTGCGGAGGC ACCTGAACCA CATGAGGGCC
TGGATGGAAG CCAGAGCAATCAACGTCAAA GGGCTCTCAA
GAAACCTGTG TAACCCAAGT TTTCCTTACG ACGCTACAAA
ACTCCGATAT TATATCTAGA CTGCTACGGC AGAGTCTGTA
GAACATACAA AGCTATCATGTCGCGTAATC TGCACAGTGC
CTGTATCCTA TTGCGATACT CCCGAAGATA TCCCTATCCG
CATACACGGA ACTGTAGACA ATTTTGCCGT GGATTGTCAA
TGTTTGTCCA ACGCGCCAGAGCTCTTCTGC GGGGACCTCC
ATCCAAGTGG TCCCAAGCTG TTTTGGTCTG TGTTGAACTT
TCTCTCCAAA CCCCATCCCG AACCCCGGAA AACTGCCCGA
TAATTACTGC CCCGCAAATGCGGCGTGTCA GAATCTCTCA
GCCTTGCGTC ATAATTTAGA TTTCGCATTT ATTTCGCGGA
TTAATAGGCC GTGGGCGTGA CTCGAACCTC ATGCACAAAA
ACGAGGATGT GCCTGAGATAGAAGGTCACC AGACGACAGA
GCTCACCATA GTAGTGCACA GCTTTATGGT CCTACACCAA
GTCCACCTCC TCAACAACGC GGGGCTATAG GTGTTTGCAA
ATTTCTTCCA TGCGTGGCTTGCAATAGTGC TTGCATGCTG
CAACAATGCA TATGGGGTAA TCAAAACGAG CACTCGCGAG
CATTCCATCA AAGGTGAGAA ATATTATTCT AAGCTTTTTC
TGGAAGCAAA CATAGGCCGCGATCACGCAC CAAAATTGAG
CCGGATTAAG AATGGGCTTC CCCCACTTCA TATTTGTGTG
GCATTAGGAC TATATATAGT TGATACATTC TCGACACACT
AGTGTCCATC ATC
pMAL11 (SEQ ID NO: 21):
GCGCCTCAAG AAAATGATGC TGCAAGAAGA ATTGAGGAAG
GAACTATTCA
TCTTACGTTGTTTGTATCAT CCCACGATCC AAATCATGTT
ACCTACGTTA GGTACGCTAG GAACTAAAAA AAGAAAAGAA
AAGTATGCGT TATCACTCTT CGAGCCAATT CTTAATTGTG
TGGGGTCCGC GAAAATTTCC GGATAAATCC TGTAAACTTT
AACTTAAACC CCGTGTTTAG CGAAATTTTC AACGAAGCGC
GCAATAAGGA GAAATATTAT CTAAAAGCGA GAGTTTAAGC
GAGTTGCAAG AATCTCTACG GTACAGATGC AACTTACTAT
AGCCAAGGTC TATTCGTATT ACTATGGCAG CGAAAGGAGC
TTTAAGGTTT TAATTACCCC ATAGCCATAG ATTCTACTCG
GTCTATCTAT CATGTAACAC TCCGTTGATG CGTACTAGAA
AATGACAACG TACCGGGCTT GAGGGACATA CAGAGACAAT
TACAGTAATC AAGAGTGTAC CCAACTTTAA CGAACTCAGT
AAAAAATAAG GAATGTCGAC ATCTTAATTT TTTATATAAA
GCGGTTTGGT ATTGATTGTT TGAAGAATTT TCGGGTTGGT
GTTTCTTTCT GATGCTACAT AGAAGAACAT CAAACAACTA
AAAAAATAGT ATAAT
pMAL12 (SEQ ID NO: 22):
ATTATACTAT TTTTTTAGTT GTTTGATGTT CTTCTATGTA
GCATCAGAAA GAAACACCAA CCCGAAAATT CTTCAAACAA
TCAATACCAA ACCGCTTTAT ATAAAAAATT AAGATGTCGA
CATTCCTTAT TTTTTACTGA GTTCGTTAAA GTTGGGTACA
CTCTTGATTA CTGTAATTGT CTCTGTATGT CCCTCAAGCC
CGGTACGTTG TCATTTTCTA GTACGCATCA ACGGAGTGTT
ACATGATAGA TAGACCGAGT AGAATCTATG GCTATGGGGT
AATTAAAACC TTAAAGCTCC TTTCGCTGCC ATAGTAATAC
GAATAGACCT TGGCTATAGT AAGTTGCATC TGTACCGTAG
AGATTCTTGC AACTCGCTTA AACTCTCGCT TTTAGATAAT
ATTTCTCCTT ATTGCGCGCT TCGTTGAAAA TTTCGCTAAA
CACGGGGTTT AAGTTAAAGT TTACAGGATT TATCCGGAAA
TTTTCGCGGA CCCCACACAA TTAAGAATTG GCTCGAAGAG
TGATAACGCA TACTTTTCTT TTCTTTTTTT AGTTCCTAGC
GTACCTAACG TAGGTAACAT GATTTGGATC GTGGGATGAT
ACAAACAACG TAAGATGAAT AGTTCCTTCC TCAATTCTTC
TTGCAGCATC ATTTTCTTGA GGCGCTCTGG GCAAGGTATA
AAAAGTTCCA TTAATACGTC TCTAAAAAAT TAAATCATCC
ATCTCTTAAG CAGTTTTTTT GATAATCTCA AATGTACATC
AGTCAAGCGT AACTAAATTA CATAA
pMAL31 (SEQ ID NO: 23):
TTATGTATTT TAGTTACGCT TGACTGATGT ACATTTGAGA
TTATCAAAAA AACTGCTTAA GAGATAGATG GTTTAATTTT
TTAGAGACGT ATTAATGGAA CTTTTTATAC CTTGCCCAGA
GCGCCTCAAG AAAATGATGC TGAAAGAAGA ATTGAGGAAG
GAACTACTCA TCTTACGTTG TTTGTATCAT CCCACGATCC
AAATCATGTT ACCTACGTTA GGTACGCTAG GAACTGAAAA
AAGAAAAGAA AAGTATGCGT TATCACTCTT CGAGCCAATT
CTTAATTGTG TGGGGTCCGC GAAAACTTCC GGATAAATCC
TGTAAACTTA AACTTAAACC CCGTGTTTAG CGAAATTTTC
AACGAAGCGC GCAATAAGGA GAAATATTAT ATAAAAGCGA
GAGTTTAAGC GAGGTTGCAA GAATCTCTAC GGTACAGATG
CAACTTACTA TAGCCAAGGT CTATTCGTAT TGGTATCCAA
GCAGTGAAGC TACTCAGGGG AAAACATATT TTCAGAGATC
AAAGTTATGT CAGTCTCTTT TTCATGTGTA ACTTAACGTT
TGTGCAGGTA TCATACCGGC CTCCACATAA TTTTTGTGGG
GAAGACGTTG TTGTAGCAGT CTCCTTATAC TCTCCAACAG
GTGTTTAAAG ACTTCTTCAG GCCTCATAGT CTACATCTGG
AGACAACATT AGATAGAAGT TTCCACAGAG GCAGCTTTCA
ATATACTTTC GGCTGTGTAC ATTTCATCCT GAGTGAGCGC
ATATTGCATA AGTACTCAGT ATATAAAGAG ACACAATATA
CTCCATACTT GTTGTGAGTG GTTTTAGCGT ATTCAGTATA
ACAATAAGAA TTACATCCAA GACTATTAAT TAACT
pMAL32 (SEQ ID NO: 24):
AGTTAATTAA TAGTCTTGGA TGTAATTCTT ATTGTTATAC
TGAATACGCT AAAACCACTC ACAACAAGTA TGGAGTATAT
TGTGTCTCTT TATATACTGA GTACTTATGC AATATGCGCT
CACTCAGGAT GAAATGTACA CAGCCGAAAG TATATTGAAA
GCTGCCTCTG TGGAAACTTC TATCTAATGT TGTCTCCAGA
TGTAGACTAT GAGGCCTGAA GAAGTCTTTA AACACCTGTT
GGAGAGTATA AGGAGACTGC TACAACAACG TCTTCCCCAC
AAAAATTATG TGGAGGCCGG TATGATACCT GCACAAACGT
TAAGTTACAC ATGAAAAAGA GACTGACATA ACTTTGATCT
CTGAAAATAT GTTTTCCCCT GAGTAGCTTC ACTGCTTGGA
TACCAATACG AATAGACCTT GGCTATAGTA AGTTGCATCT
GTACCGTAGA GATTCTTGCA ACCTCGCTTA AACTCTCGCT
TTTATATAAT ATTTCTCCTT ATTGCGCGCT TCGTTGAAAA
TTTCGCTAAA CACGGGGTTT AAGTTTAAGT TTACAGGATT
TATCCGGAAG TTTTCGCGGA CCCCACACAA TTAAGAATTG
GCTCGAAGAG TGATAACGCA TACTTTTCTT TTCTTTTTTC
AGTTCCTAGC GTACCTAACG TAGGTAACAT GATTTGGATC
GTGGGATGAT ACAAACAACG TAAGATGAGT AGTTCCTTCC
TCAATTCTTC TTTCAGCATC ATTTTCTTGA GGCGCTCTGG
GCAAGGTATA AAAAGTTCCA TTAATACGTC TCTAAAAAAT
TAAACCATCT ATCTCTTAAG CAGTTTTTTT GATAATCTCA
AATGTACATC AGTCAAGCGT AACTAAAATA CATAA
pMAL13 (SEQ ID NO: 25):
AGTACAGGAACGATTGTCTTGATAATATGTGAAAAGTGCACACG
AAATTAGAGGGTGTCCTTTACAAGTATTCTTAGAAACACATTCA
AGAGCACAAAAGTCGATGCTTTAAGGGTCAAGGTGGTGGAAAAC
TTGACTGGAATTCTTGACGAAAAAACAAGAAAAACGTGATTCGA
GCAATCATAAACATACAGCCCCGTTCCAACCGGATCTTGAGGTT
TCCCATTTTAGATGGAAATAAGCAGAGCAAAATAAAAATCTTGA
ACAAGTAATAGTGGTGACTGCAGGTTACGTTGGCATATAAAGTC
CGGGTGACCTGGGTTTCCTGCACCACCAGCCCCCATATGCTAGC
ACAATGGGTTTTCTTTATCCCCGGTCATAATTACTCATTTTGCT
ATATTCTTCATAACTTAAGTACGCAGATAGAGAAAATTAATAAT
CTCGATATATATTAAAGTAAATGAAAAGTAGAAAATTTAGCCAG
AACTCTTTTTTGCTTCGAGT
pMAL22 (SEQ ID NO: 26):
GTAGCTTCACTGCTTGGATACCAATACGAATAGACCTTGGCTAT
AGTAAGTTGCATCTGTACCGTAGAGATTCTTGCAACCTCGCTTA
AACTCTCGCTTTTATATAATATTTCTCCTTATTGCGCGCTTCGT
TGAAAATTTCGCTAAACACGGGGTTTAAGTTTAAGTTTACAGGA
TTTATCCGGAAGTTTTCGCGGACCCCACACAATTAAGAATTGGC
TCGAAGAGTGATAACGCATACTTTTCTTTTCTTTTTTCAGTTCC
TAGCGTACCTAACGTAGGTAACATGATTTGGATCGTGGGATGAT
ACAAACAACGTAAGATGAGTAGTTCCTTCCTCAATTCTTCTTTC
AGCATCATTTTCTTGAGGCGCTCTGGGCAAGGTATAAAAAGTTC
CATTAATACGTCTCTAAAAAATTAAACCATCTATCTCTTAAGCA
GTTTTTTTGATAATCTCAAATGTACATCAGTCAAGCGTAACTAA
AATACATAA
pMAL33 (SEQ ID NO: 27):
AGCTCAGTTGTCAAGATTTAGTCATTAAGAAGGGCCGCAGCAGC
TTTTTGTATAATAGAGCGTCTTTTTTGTTTGTGAAAAAAATTTT
ATGGTGAGATATTGTTCGATTCTACGAAGTCATTTTACTAGTTT
ATGGACTCTGATATAAGACAGAGTTGACAAGGAAATGGTGCCGT
GATTGTTTCCGTGTACAGCTTTTGAGAACTTCCTTGAAAACCAA
TCATCTAGCACTTTCATTTCTGGGGAAAAACCTGGAACCAAATC
TTGAAAAATAAATTCCCCAGAAGTTTTCCTTATTCCGTGTTCTA
ATCTTCTCGTTCACTTTGCAGTGACATTCCACGGCCATGCGCAA
TTTACCCCGCCCCCGGATTTTATTGTCCGTACCGCCATTTTTCA
ATAGATTAAAAAGGAACAAAAAATCATTTCAGAAGGTTTCTTTC
TCGGGAAAACACTAGAGTGTAAATATTGAATATCAAACATCGAA
CGAGAGCATCTTGAAGATATTTATGTTCTAAAT
pCAT8 (SEQ ID NO: 28):
GTGTTTATTCGCGATATGAGTTGTGATATCAGAGACAGAGAGAG
TTTATGTGCGTAACAGGAACGGAGAAAACCAGAGTAATTGAGTA
TTATAAGCAATAAATCATAAAAAGACATTCTTTCTCGTGCAATT
TTTTGGTATTCGGGATAATCTTCTACTTGAAACTTCTTTTTTTC
GGTGTTTAATTTGCCTATTGGTAAATATTTTTGCCGCCGAGGTT
CTCAGTGATTATATTCGTATTAAGCGATAACCGAGACATGCATG
GAGCGGCGGGGCTGATATTTTGTGGGGTACGAAAGCATGATTGG
TCAGTGACACTCAAAAAAAGAAAACAGCCGTAAAATAGTAGATT
TTGTTAAACTCCCCTTTAAACCTGTGATATTGTAAAAAGACGAA
GAATTTAATAATTTAATAATTCATTACGGTATTTATTTCTTCAT
AAACAGTTACAACACCCTAAAGAGAATTTACAAGTTGAGTAAAA
GACAAGACACAAAATT
pHXT2 (SEQ ID NO: 29):
CGCAGCTTCACTTTTAAGTTTCTTTTTCTCCTCACGGCGCAACC
GCTAACTTAAGCTAATCCTTATGAATCCGGAGAAAAGCGGGGTC
TTTTAACTCAATAAAATTTTCCGAAATCCTTTTTCCTACGCGTT
TTCTTCGGGAACTAGATAGGTGGCTCTTCCACCTGTTTTTCCAT
CATTTTAGTTTTTCGCAAGCCATGCGTGCCTTTTCGTTTTTGCG
ATGGCGAAGCAGGGCTGGAAAAATTAACGGTACGCCGCCTAACG
ATAGTAATAGGCCACGCAACTGGCGTGGACGACAACAATAAGTC
GCCCATTTTTTATGTTTTCAAAACCTAGCAACCCCCACCAAACT
TGTCATCGTTCCCGGATTCACAAATGATATAAAAAGCGATTACA
ATTCTACATTCTAACCAGATTTGAGATTTCCTCTTTCTCAATTC
CTCTTATATTAGATTATAAGAACAACAAATTAAATTACAAAAAG
ACTTATAAAGCAACATA
pHXT4 (SEQ ID NO: 30):
CGTCTCTTTCTGTGGAGAAGAAGATATTTCCCCGAGCAGTTTTT
TTTCCATGGGGCCCCATATTCCCCCGCCTGCAGGAAAACTTGGG
GAAAGAGGAAAAACACTTCGGATAAAAACGGTCAAGAAGCTCTT
CGACGATTTAGTGCCACCTTCATGAAAAATTCCAGAGTTTTTTC
CAGCTGCTTTGATTTTACAGTCCATTATTCGGCGTCTAACGATT
CTGATTAAGAAACAACGGAGGAAAACTCAAATTCTAATATAATA
TTTTTAAGTTTATGAAGGTGGGGTGGTAAGAAAAGCAACTAAAA
TAATCTACAAGTCAATTAGTGGTGAAAAGCTTCAACACTGGGGA
ATGAATAATATGTCATCTAGAAAAAATTTTATATAAATACTCAG
TGTTTTATTCATTATTCTCGATTCATTCACTTCAATTCCTCTTC
ATGAGTAATAGAAACCATCAAGAAAAGATATATTCAAAGCCTCT
TATCAAGGTTTGGTTTTGAAACACTTTTACAATAAAATCTGCCA
AAA
pMTH1 (SEQ ID NO: 31):
TCCGAAATTATTCCCTAGAACAAGCGGGAAAAAGGTCCGGGGAA
ATGGAGTCCGTGCGAGTTTTGTTAGGATGACTGCCCCACACATT
TCCTCATCTTATAATTTTGTGGAAAAATTCATCGTGAGAGAAAA
TACGAGTCCATTTCTCCAGTGAAACTACCGTAGACATGGAATAT
CTGCCATTCTACCCCTTATTCAAGTGCCTTTTTTTTTTTTTTTC
ATCCCACATTTTATTGCTGCCTCAATCTCCATTAAGAAAAAAAA
TTTATATAACCAAATGACATTTTTCCTTTCTTCTCAAACTTTGT
AATGCGCCTGTAACTGCTTCTTTTTTTATTAAAAAACAGCATGG
AGTTTTTTAATAACTTAAGGAAACATACAAAAAGATTTGTTCAT
TTCACTCCAAGTATTTTTTAACGTATATTGAAAGTTCTCAATAG
CGAAACCACAAGCAGCAATACAAAGAGAATTTTATTCGAACGCA
TAGAGTACACACACTCAAAGGA
pSUC2 (SEQ ID NO: 32):
CATTATGAGGGCTTCCATTATTCCCCGCATTTTTATTACTCTGA
ACAGGAATAAAAAGAAAAAACCCAGTTTAGGAAATTATCCGGGG
GCGAAGAAATACGCGTAGCGTTAATCGACCCCACGTCCAGGGTT
TTTCCATGGAGGTTTCTGGAAAAACTGACGAGGAATGTGATTAT
AAATCCCTTTATGTGATGTCTAAGACTTTTAAGGTACGCCCGAT
GTTTGCCTATTACCATCATAGAGACGTTTCTTTTCGAGGAATGC
TTAAACGACTTTGTTTGACAAAAATGTTGCCTAAGGGCTCTATA
GTAAACCATTTGGAAGAAAGATTTGACGACTTTTTTTTTTTGGA
TTTCGATCCTATAATCCTTCCTCCTGAAAAGAAACATATAAATA
GATATGTATTATTCTTCAAAACATTCTCTTGTTCTTGTGCTTTT
TTTTTACCATATATCTTACTTTTTTTTTTCTCTCAGAGAAACAA
GCAAAACAAAAAGCTTTTCTTTTCACTAACGTATATG

1. A host cell comprising a heterologous genetic pathway that produces a heterologous product and that is regulated by an exogenous agent, wherein the host cell does not produce a precursor required to make the product.

2. The host cell of claim 1, wherein the exogenous agent comprises a regulator of gene expression.

3. The host cell of claim 2, wherein the exogenous agent decreases production of the heterologous product, or wherein the exogenous agent is glucose and expression of one or more enzymes encoded by the heterologous genetic pathway are under control of a glucose repressed promoter.

4. (canceled)

5. The host cell of claim 2, wherein the exogenous agent increases production of the heterologous product, or wherein the exogenous agent is galactose and expression of one or more enzymes encoded by the heterologous genetic pathway are under control of a GAL promoter.

6. (canceled)

7. The host cell of claim 1, wherein the heterologous genetic pathway comprises a galactose-responsive promoter, a maltose-responsive promoter, or a combination of both, or the heterologous product is a cannabinoid or cannabinoid precursor, wherein the cannabinoid or cannabinoid precursor is CBDA, CBD, CBGA, or CBG.

8. (canceled)

9. (canceled)

10. The host cell of claim 7, wherein the genetic pathway encodes at least two enzymes selected from the group consisting of hexanoyl-CoA synthase (HCS), tetraketide synthase (TKS) and olivetolic acid cyclase (OAC).

11. The host cell of claim 1, wherein the precursor required to make the product is hexanoate, olivetol, or olivetolic acid.

12. The host cell of claim 1, wherein the heterologous genetic pathway comprises a nucleic acid construct comprising at least 3 protein coding regions.

13. The host cell of claim 1, wherein the host cell is a yeast cell or yeast strain, or the host cell is S. cerevisiae.

14. (canceled)

15. A mixture comprising the host cell of claim 1 and a culture media.

16. The mixture of claim 15, wherein the culture media comprises an exogenous agent that decreases production of the heterologous product, or wherein the culture media comprises glucose, maltose, or lysine.

17. (canceled)

18. The mixture of claim 15, wherein the culture media comprises (i) an exogenous agent that increases production of the heterologous product, and (ii) a precursor required to make the heterologous product, or the culture media comprises galactose and hexanoate.

19. (canceled)

20. (canceled)

21. A method for decreasing the expression of a heterologous product, comprising culturing the host cell of claim 1 in a media comprising an exogenous agent, wherein the exogenous agent decreases the expression of the heterologous product, or the media comprises glucose, maltose, or lysine, or wherein culturing the host cell in the media comprising the exogenous agent results in less than 0.001 mg/L of heterologous product.

22. (canceled)

23. (canceled)

24. A method for increasing the expression of a heterologous product, comprising (i) culturing the host cell of claim 1 in a media comprising the exogenous agent, wherein the exogenous agent increases expression of the heterologous product, or (ii) culturing the host cell of claim 1 in a media comprising galactose.

25. (canceled)

26. The method of claim 24, further comprising (i) culturing the host cell with the precursor required to make the heterologous product, or (ii) culturing the host cell with hexanoate.

27. (canceled)

28. A host cell comprising a heterologous genetic pathway that produces a cannabinoid and is regulated by an exogenous agent, wherein the host cell does not comprise hexanoate at a level to make the cannabinoid in an amount over 10 mg/L.

29. The host cell of claim 28, wherein (i) the exogenous agent downregulates expression of the heterologous genetic pathway, or
(ii) the exogenous agent is glucose and expression of one or more enzymes encoded by the heterologous genetic pathway are under control of a glucose repressed promoter; or
(iii) the exogenous agent upregulates expression of the heterologous genetic pathway, or
(iv) the exogenous agent is galactose and expression of one or more enzymes encoded by the heterologous genetic pathway are under control of a GAL promoter; or
(v) wherein the genetic pathway encodes at least two enzymes selected from the group consisting of hexanoyl-CoA synthase (HCS), tetraketide synthase (TKS) and olivetolic acid cyclase (OAC); or
(vi) the host cell is a yeast cell or yeast strain, or the host cell is S. cerevisiae.

30. (canceled)

31. (canceled)

32. (canceled)

33. (canceled)

34. (canceled)

35. (canceled)

36. A method for decreasing expression of a cannabinoid, comprising (i) culturing the host cell of claim 28 in a media comprising the exogenous agent, wherein the exogenous agent decreases the expression of the cannabinoid or a precursor thereof; or (ii) culturing the host cell of claim 28 in a media comprising glucose, maltose, or lysine; wherein culturing the host cell in the media comprising the exogenous agent results in less than 0.001 mg/L of cannabinoid or a precursor thereof.

37. (canceled)

38. (canceled)

39. A method for increasing expression of a cannabinoid, comprising (i) culturing the host cell of claim 28 in a media comprising the exogenous agent, wherein the exogenous agent increases the expression of the cannabinoid or a precursor thereof; or (ii) culturing the host cell of claim 28 in a media comprising galactose.

40. (canceled)

41. The method of claim 39, further comprising culturing the host cell in a media comprising hexanoate.

42. The method of claim 36, wherein the cannabinoid is CBDA, CBD, CBGA, or CBG.

43. The method of claim 36, wherein the host cell is a yeast cell or yeast strain, or the host cell is S. cerevisiae.

44. (canceled)


SEQUENCE LISTING (32 sequences)

SEQ ID NO: 1 - DNA, 6742 bp, Artificial Sequence
  source: /note=“Description of Artificial Sequence Synthetic polynucleotide”
gacggcacggccacgcgtttaaaccgcctacgccatcattaaagacctggtcaactataaaataatacaatcaatacttgcttgaacgcttgattttactgatattctatccaaaagcaagtagaccagaaactctcaagatgttgcaaataccgttcgatgtttttggtttagattgttttaatgttgatgcttttttacttatttttggaagcgtctttttaatttagttttatattataggtatatgaatgtgtttatgccaataagggtttttttgtacagttatgtgattataaacagtcttttgtctagtttttttcaccagtatcggcctctatttataaaaaacggagcagctttcggtgtcagtaattctgaaaaaatttgtgtcactctgattgtaaatgaattaatttagctagatagttgcgagccccaacgagaagattgtcagacaaagacaacattcaacaacctacatccgttactattcgttaactcgaggtacttgaaacttttcagttaagtcgctcgtccaacgccggcggacctgcgagtaagcaactctggcgctggcatggcataaccggcgacggcaatgcgcaagatgggatgctatgggcagagagccgtactttactgcttatggcactacagcaacagatggttaccccactaagcctgaagcgaatcgccatcaattctgcgcagtggcgaggagataaaagcgcggaagtcattcatcaactggcgacgctactcaaagcagggttaacgctttctgaagggctggctctgctggcggaacagcatcccagtaagcaatggcaagcgttgctgcaatcgctggcgcacgatctcgaacagggcattgctttttccaatgccttattaccctggtcagaggtatttccgccgctctatcaggcgatgatccgcacgggtgaactgaccggtaagctggatgaatgctgctttgaactggcgcgtcagcaaaaagcccagcgtcagttgaccgacaaagtgaaatcagcgttacgttatcccatcatcattttagcgatggcaatcatggtggttgtggcaatgctgcattttgttctgccggagtttgccgctatctataagaccttcaacaccccactaccggcactaacgcaggggatcatgacgctggcagactttagtggcgaatggagctggctgctggtgttgttcggctttctgctggcgatagccaataagttgctgaacggccggccaagcacgcggggatcagtaggacaaagggttctcgtagagtccccggaaaaaaaaaaggacaaaaagtttcaagacggcaatctctttttactgcatctcgtcagttggcaacttgccaagaacttcgcaaatgactttgacatatgataagacgtcaactgccccacgtacaataacaaaatggtagtcatattatgtcaagaataggtatccaaaacgcagcggttgaaagcatatcaagaattgtgtccctgtgtttcaaagtttgtggataatcgaaatctcttacattgaaaacattatcatacaatcatttattaagtagttgaagcatgtatgaactataaaagtgttactactcgttattattgtgtactttgtgatgctaaagttatgagtagaaaaaaatgagaagttgttctgaacaaagtaaaaaaaagaagtatacttattcaaaatgggagaattgttgacgcaaaactctacgcatgatcttgttggtggcagttctaggcaaagaagacaaagggacgactctagtaaccttaaacaatggattcaacttcttttgcaaacccaagttgaaggacaatctcaattggttcaagtcgatagtagtatcgttagaatccttcaagacgaagaaaataaccaattgttctggaccaccacctaatggtggaacaccgatagcagtggtttcgaaaactctgtcatcgacttcgttacaaactctttcaatctcaatggaagagattttgataccaccgatgttcatggtgtcatcagcacgaccgtgagcatggtagtaaccgttggaagttaattcaaagatgtcaccgtgtcttctcaaaacttcaccgttcaaagttggcatacctttgaagtagacatcgtggtggttaccgttcaataaagtcttagaagcaccgaacataactggacccaaagccaattcaccaatacctggcttgttctttggcattgggtaaccgttcttatccaaaatgtacaaagtacaacccatacattgggaagaaaaggaggacaaggattgggcttgtaagaaagaaccagcagagaaagcaccaccgatttcggtaccaccacacatttcgataacaggtttatagttggctctacccatcaaccacaagtattcatcgacgttagaagcttcaccagaggaagaaaagcaacggatggtagaccagtcataaccggaaacgcagttggtggatttccaagatctaacaatagatggaacaacacctaacatagtaaccttagcgtcttggacgaacttggcgaaaccagaaaccaatggggaaccattatacaaagcgatagaagcaccgttcaataaagaggcgtaaaccaaccatggacccatcatccaacctaaattagttggccaaacaatgacgtcacctttacgaatatccaagtgagaccaaccgtcggcagcagccttcaatggagtagcttgggtccatggaatggcctttggttcaccagtggtaccggaagagaataaaatgttggtgtaggcatcaactggttgttcacgagcggtgaattcacagttcttgaattccttagcacgttccaagaaataatcccaggaaatgtcaccgtcacgcaattcggcaccgatgttggaaccggaacatggaatgacaatagccattggagacttagcttcaacgactctagaatacaatggaattctcttcttaccacggatgatgtggtcttgagtgaagatggccttagccttagacaatctcaatctagtagagatttctggagcggagaaagaatcagcgatggaaacgacgacgtaaccagccaagacaatggctaagtagatgacgacagcgtcaacgtgcattggcatatcgatggcgatagcacaacctttttccaaacccatttcttccaaggcataaccaaccaaccaaactctctttctcaattggtccaaagtcaacttgttcaatggcaaatcgtcgttaccctcatcacgccaaacgatcatagtatcattcaactttttgttagagttaacattcaagcagttcttagcagagttcaagtaaccacctggcaaccattcggaaccacctgggttgttaatatcgtctctacgtaagatacattctggatctttagaaaaggagatcttcatttcatccattaaaacagttctccagtaaacttctgggtttctgacggagaactcttggaagtgagaaaaagaagaaattggatccttgtatttaacacccaagaattccttacctcttttctccaacaaagcacccaagttggtagacttgaccttttcagggtctggaatccaagctggtggggctggaccaaagtccttgtaacaaccatagaataacatttggtgcaaggaaaatggcaagtctggggataagatatggttggcaatgttaatccaagtttgtggggtagcagcaccgtaattacaaacaatttcagctaatctaccatgcaaagtttcggcgacctcagaggtaatacccaaagcgatgaaatcggaagcaacaacagaatccaaagatttgtagttcttacccattatagttttttctccttgacgttaaagtatagaggtatattaacaattttttgttgatacttttatgacatttgaataagaagtaatacaaaccgaaaatgttgaaagtattagttaaagtggttatgcagcttttgcatttatatatctgttaatagatcaaaaatcatcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataaatgaaccacttaagagctgaaggtccagcttccgttttggccattggtaccgctaacccagaaaacatcttgttgcaagacgaatttccagactactacttcagagtcactaagtccgaacacatgacccaattgaaggaaaagttcagaaagatttgtgataagtctatgatcagaaaaagaaactgtttcttgaacgaagaacacttgaaacaaaaccctagattagttgaacatgaaatgcaaactttagatgccagacaagatatgttggtcgtcgaagtcccaaagttgggtaaggacgcttgtgccaaggctatcaaggaatggggtcaaccaaagtctaagattactcatttgatcttcacttccgcctctaccaccgatatgccaggtgctgattaccattgtgctaagttgttgggtttatccccatctgttaaaagagttatgatgtaccaattgggttgttatggtggtggtactgttttgagaattgccaaagacatcgctgaaaacaataagggtgctagagttttggctgtttgttgtgatattatggcttgtttgttcagaggtccatccgagtctgatttagagttgttagttggtcaagctattttcggtgacggtgctgctgctgttattgttggtgctgaaccagacgaatctgttggtgaacgtccaatctttgaattggtctctaccggtcaaaccatcttgccaaactctgaaggtaccattggtggtcacatcagagaagctggtttgatcttcgatttgcataaagatgttcctatgttgatttctaataacatcgaaaagtgcttaatcgaagctttcactccaatcggtatctctgattggaattccattttctggattacccatccaggtggtaaggccatcttggataaggttgaagaaaagttgcatttaaagtctgataagttcgttgactctcgtcacgttttgtctgaacatggtaacatgtcttcttccactgttttgtttgttatggatgaattgagaaaaagatccttggaagaaggtaagtctactactggtgatggttttgaatggggtgtcttgttcggttttggtccaggtttgaccgttgaaagagttgtcgttagatccgttccaatcaagtactaatttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaagattgaaaatctatggaaagatatggacggtagcaacaagaatatagcacgagccgcgaagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctaggtccgccggcgttggacgagcgaaaattcatttaatattcaatgaagttataaattgatagtttaaataaagtcagtctttttcctcatgtttagaattgtattaatgtacgccgtttacgttggagtgtaaatgtgtctattccagaacgaaatctaaatgagcagtacaggcagttcagatggtactgaagcggtactaaagatgcatgaattgaacagatgtggtagttatgtatatgaggaatatgagttgtcacattaaaaatataatagctatgatcccattattatattcgtgacagttcgtaacgttttaattggcttatgtttttgagaaatgggtgaattttaagataattgttgggattccattattgataaaggctataatattaggtatacagaatatactggaagttctcctcgaggatataggaatcctcaaaatggaatctatatttctatttactaatatcacgattattcttcattccgttttatatgtttcattatcctattacattatcaatccttgcatttcagcttcctctaacttcgatgacagctggcggtttaaacgcgtggccgtgccgtc

SEQ ID NO: 2 - DNA, 5782 bp, Artificial Sequence
  source: /note=“Description of Artificial Sequence Synthetic polynucleotide”
gacggcacggccacgcgtttaaaccgccagagtatgtcaactggcgcagtagatacatgtttttctcttccacgtcgaattttgttatatacatagcataatcgagttgtatgcaccctttttgtttatctcgttagtaactcggggtaggaataagacatccacaaaggtgacagaacaaaatcatcctagccttgttcataatctacctctatatagccgctaaaaaattagtagtattttgactctttaagagcacatttattatcaggctgcttttacatacttcttttgtttaaaacatttaaagacgatcactgcccttccaaaggacaaatatatatacacaaacactaggccaaaagttcacttataataatttagtggtaattatgttgggtaaagaaattgccaatagtctttttttttccgtattgtaaggtgagactgaggtagcggcacaaaaaaacgacacataataggatactgagtaaagcagtattaaaataaaaagatatattttacctcgaacgctacaaataaagcagaaaagaacaaaatcgtgagccgctcgtccaacgccggcggacctagctttccaaaaggagaggcaaagcaatttaagaatgtatgaacaaaataaaggggaaaaattaccccctctactttaccaaacgaatactaccaataatatttacaacttttccttatgattttttcactgaagcgcttcgcaatagttgtgagcgatatcaaaagtaacgaaatgaacttcgcggctcgtgctatattcttgttgctaccgtccatatctttccatagattttcaatctttgatgtctccatggtggtacagagaacttgtaaacaattcggtccctacatgtgaggaaattcgctgtgacacttttatcactgaactccaaatttaaaaaatagcataaaattcgttatacagcaaatctatgtgttgcaattaagaactaaaagatatagagtgcatattttcaagaaggatagtaagctggcaaattagtacttgattggaacggatctaacgacaactctttcaacggtcaaacctggaccaaaaccgaacaagacaccccattcaaaaccatcaccagtagtagacttaccttcttccaaggatctttttctcaattcatccataacaaacaaaacagtggaagaagacatgttaccatgttcagacaaaacgtgacgagagtcaacgaacttatcagactttaaatgcaacttttcttcaaccttatccaagatggccttaccacctggatgggtaatccagaaaatggaattccaatcagagataccgattggagtgaaagcttcgattaagcacttttcgatgttattagaaatcaacataggaacatctttatgcaaatcgaagatcaaaccagcttctctgatgtgaccaccaatggtaccttcagagtttggcaagatggtttgaccggtagagaccaattcaaagattggacgttcaccaacagattcgtctggttcagcaccaacaataacagcagcagcaccgtcaccgaaaatagcttgaccaactaacaactctaaatcagactcggatggacctctgaacaaacaagccataatatcacaacaaacagccaaaactctagcacccttattgttttcagcgatgtctttggcaattctcaaaacagtaccaccaccataacaacccaattggtacatcataactcttttaacagatggggataaacccaacaacttagcacaatggtaatcagcacctggcatatcggtggtagaggcggaagtgaagatcaaatgagtaatcttagactttggttgaccccattccttgatagccttggcacaagcgtccttacccaactttgggacttcgacgaccaacatatcttgtctggcatctaaagtttgcatttcatgttcaactaatctagggttttgtttcaagtgttcttcgttcaagaaacagtttctttttctgatcatagacttatcacaaatctttctgaacttttccttcaattgggtcatgtgttcggacttagtgactctgaagtagtagtctggaaattcgtcttgcaacaagatgttttctgggttagcggtaccaatggccaaaacggaagctggaccttcagctcttaagtggttcatttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactataatgaaccacttaagagctgaaggtccagcttccgttttggccattggtaccgctaacccagaaaacatcttgttgcaagacgaatttccagactactacttcagagtcactaagtccgaacacatgacccaattgaaggaaaagttcagaaagatttgtgataagtctatgatcagaaaaagaaactgtttcttgaacgaagaacacttgaaacaaaaccctagattagttgaacatgaaatgcaaactttagatgccagacaagatatgttggtcgtcgaagtcccaaagttgggtaaggacgcttgtgccaaggctatcaaggaatggggtcaaccaaagtctaagattactcatttgatcttcacttccgcctctaccaccgatatgccaggtgctgattaccattgtgctaagttgttgggtttatccccatctgttaaaagagttatgatgtaccaattgggttgttatggtggtggtactgttttgagaattgccaaagacatcgctgaaaacaataagggtgctagagttttggctgtttgttgtgatattatggcttgtttgttcagaggtccatccgagtctgatttagagttgttagttggtcaagctattttcggtgacggtgctgctgctgttattgttggtgctgaaccagacgaatctgttggtgaacgtccaatctttgaattggtctctaccggtcaaaccatcttgccaaactctgaaggtaccattggtggtcacatcagagaagctggtttgatcttcgatttgcataaagatgttcctatgttgatttctaataacatcgaaaagtgcttaatcgaagctttcactccaatcggtatctctgattggaattccattttctggattacccatccaggtggtaaggccatcttggataaggttgaagaaaagttgcatttaaagtctgataagttcgttgactctcgtcacgttttgtctgaacatggtaacatgtcttcttccactgttttgtttgttatggatgaattgagaaaaagatccttggaagaaggtaagtctactactggtgatggttttgaatggggtgtcttgttcggttttggtccaggtttgaccgttgaaagagttgtcgttagatccgttccaatcaagtactaagtatacttcttttttttactttgttcagaacaacttctcatttttttctactcataactttagcatcacaaagtacacaataataacgagtagtaacacttttatagttcatacatgcttcaactacttaataaatgattgtatgataatgttttcaatgtaagagatttcgattatccacaaactttgaaacacagggacacaattcttgatatgctttcaaccgctgcgttttggatacctattcttgacataatatgactaccattttgttattgtacgtggggcagttgacgtcttatcatatgtcaaagtcatttgcgaagttcttggcaagttgccaactgacgagatgcagtaaaaagagattgccgtcttgaaactttttgtccttttttttttccggggactctacgagaaccctttgtcctactgatccccgcgtgcttggccggccgtgatcatctacccatgccgaaattcgggccgttggccggattgcgcgttgtcttctccggtatcgaaatcgccggaccgtttgccgggcaaatgttcgcagaatggggcgcggaagttatctggatcgagaacgtcgcctgggccgacaccattcgcgttcaaccgaactacccgcaactctcccgccgcaatttgcacgcgctgtcgttaaatattttcaaagatgaaggccgcgaagcgtttctgaaattaatggaaaccaccgatatcttcatcgaagccagtaaaggtccggcctttgcccgtcgtggcattaccgatgaagtactgtggcagcacaacccgaaactggttatcgctcacctgtccggttttggtcagtacggcaccgaggagtacaccaatcttccggcctataacactatcgcccaggcctttagtggttacctgattcagaacggtgatgttgaccagccaatgcctgccttcccgtataccgccgattacttttctggcctgaccgccaccacggcggcgctggcagcactgcataaagtgcgtgaaaccggtaaaggcgaaagtatcgacatcgccatgtatgaagtgatgctgcgtatgggccagtacttcatgatggattacttcaacggcggcgaaatgtgcccgcgcatgagcaaaggtaaagatccctactacgccgaggtccgccggcgttggacgagcgactttaatgtcgttctccctttttaaagagtaaatacatatttaaaaaagtgactatggctattgctaaacgtgataaaaatcagagcctataacactctctgaaataacgctatgcaggaatttccagttaagttcttcttggggtgacttctttactcggtatgatatgtgttttatatgcacagtacgagtccattagggtaaattagtggccgagaaacttttgccgccgagcttttaagtatccttttgccacttcttatttagataaagacctggcagtagtagtcgtagaagataagatagacagagaatgaatactaataagatagcacaagacgaagtccaagataaggttttgcaaagagcagaactagcacattctgtatggaacttaaggttcaacctcagtaaagttgccaaacggattcgcatggaaacaaaggtatttccagagataaagataaatgacgcgcaatcacagttagagcgatctaggtgtagaatatttagccctgacctggaggaagaacatgtgcccttgattcaaggcggcggtttaaacgcgtggccgtgccgtc

SEQ ID NO: 3 - DNA, 5737 bp, Artificial Sequence
  source: /note=“Description of Artificial Sequence Synthetic polynucleotide”
gacggcacggccacgcgtttaaaccgcctacgccatcattaaagacctggtcaactataaaataatacaatcaatacttgcttgaacgcttgattttactgatattctatccaaaagcaagtagaccagaaactctcaagatgttgcaaataccgttcgatgtttttggtttagattgttttaatgttgatgcttttttacttatttttggaagcgtctttttaatttagttttatattataggtatatgaatgtgtttatgccaataagggtttttttgtacagttatgtgattataaacagtcttttgtctagtttttttcaccagtatcggcctctatttataaaaaacggagcagctttcggtgtcagtaattctgaaaaaatttgtgtcactctgattgtaaatgaattaatttagctagatagttgcgagccccaacgagaagattgtcagacaaagacaacattcaacaacctacatccgttactattcgttaactcgaggtacttgaaacttttcagttaagtcgctcgtccaacgccggcggacctgcgagtaagcaactctggcgctggcatggcataaccggcgacggcaatgcgcaagatgggatgctatgggcagagagccgtactttactgcttatggcactacagcaacagatggttaccccactaagcctgaagcgaatcgccatcaattctgcgcagtggcgaggagataaaagcgcggaagtcattcatcaactggcgacgctactcaaagcagggttaacgctttctgaagggctggctctgctggcggaacagcatcccagtaagcaatggcaagcgttgctgcaatcgctggcgcacgatctcgaacagggcattgctttttccaatgccttattaccctggtcagaggtatttccgccgctctatcaggcgatgatccgcacgggtgaactgaccggtaagctggatgaatgctgctttgaactggcgcgtcagcaaaaagcccagcgtcagttgaccgacaaagtgaaatcagcgttacgttatcccatcatcattttagcgatggcaatcatggtggttgtggcaatgctgcattttgttctgccggagtttgccgctatctataagaccttcaacaccccactaccggcactaacgcaggggatcatgacgctggcagactttagtggcgaatggagctggctgctggtgttgttcggctttctgctggcgatagccaataagttgctgaacggccggccaagcacgcggggatcagtaggacaaagggttctcgtagagtccccggaaaaaaaaaaggacaaaaagtttcaagacggcaatctctttttactgcatctcgtcagttggcaacttgccaagaacttcgcaaatgactttgacatatgataagacgtcaactgccccacgtacaataacaaaatggtagtcatattatgtcaagaataggtatccaaaacgcagcggttgaaagcatatcaagaattgtgtccctgtgtttcaaagtttgtggataatcgaaatctcttacattgaaaacattatcatacaatcatttattaagtagttgaagcatgtatgaactataaaagtgttactactcgttattattgtgtactttgtgatgctaaagttatgagtagaaaaaaatgagaagttgttctgaacaaagtaaaaaaaagaagtatacttagtacttgattggaacggatctaacgacaactctttcaacggtcaaacctggaccaaaaccgaacaagacaccccattcaaaaccatcaccagtagtagacttaccttcttccaaggatctttttctcaattcatccataacaaacaaaacagtggaagaagacatgttaccatgttcagacaaaacgtgacgagagtcaacgaacttatcagactttaaatgcaacttttcttcaaccttatccaagatggccttaccacctggatgggtaatccagaaaatggaattccaatcagagataccgattggagtgaaagcttcgattaagcacttttcgatgttattagaaatcaacataggaacatctttatgcaaatcgaagatcaaaccagcttctctgatgtgaccaccaatggtaccttcagagtttggcaagatggtttgaccggtagagaccaattcaaagattggacgttcaccaacagattcgtctggttcagcaccaacaataacagcagcagcaccgtcaccgaaaatagcttgaccaactaacaactctaaatcagactcggatggacctctgaacaaacaagccataatatcacaacaaacagccaaaactctagcacccttattgttttcagcgatgtctttggcaattctcaaaacagtaccaccaccataacaacccaattggtacatcataactcttttaacagatggggataaacccaacaacttagcacaatggtaatcagcacctggcatatcggtggtagaggcggaagtgaagatcaaatgagtaatcttagactttggttgaccccattccttgatagccttggcacaagcgtccttacccaactttgggacttcgacgaccaacatatcttgtctggcatctaaagtttgcatttcatgttcaactaatctagggttttgtttcaagtgttcttcgttcaagaaacagtttctttttctgatcatagacttatcacaaatctttctgaacttttccttcaattgggtcatgtgttcggacttagtgactctgaagtagtagtctggaaattcgtcttgcaacaagatgttttctgggttagcggtaccaatggccaaaacggaagctggaccttcagctcttaagtggttcattatagttttttctccttgacgttaaagtatagaggtatattaacaattttttgttgatacttttatgacatttgaataagaagtaatacaaaccgaaaatgttgaaagtattagttaaagtggttatgcagcttttgcatttatatatctgttaatagatcaaaaatcatcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataaatgaaccacttaagagctgaaggtccagcttccgttttggccattggtaccgctaacccagaaaacatcttgttgcaagacgaatttccagactactacttcagagtcactaagtccgaacacatgacccaattgaaggaaaagttcagaaagatttgtgataagtctatgatcagaaaaagaaactgtttcttgaacgaagaacacttgaaacaaaaccctagattagttgaacatgaaatgcaaactttagatgccagacaagatatgttggtcgtcgaagtcccaaagttgggtaaggacgcttgtgccaaggctatcaaggaatggggtcaaccaaagtctaagattactcatttgatcttcacttccgcctctaccaccgatatgccaggtgctgattaccattgtgctaagttgttgggtttatccccatctgttaaaagagttatgatgtaccaattgggttgttatggtggtggtactgttttgagaattgccaaagacatcgctgaaaacaataagggtgctagagttttggctgtttgttgtgatattatggcttgtttgttcagaggtccatccgagtctgatttagagttgttagttggtcaagctattttcggtgacggtgctgctgctgttattgttggtgctgaaccagacgaatctgttggtgaacgtccaatctttgaattggtctctaccggtcaaaccatcttgccaaactctgaaggtaccattggtggtcacatcagagaagctggtttgatcttcgatttgcataaagatgttcctatgttgatttctaataacatcgaaaagtgcttaatcgaagctttcactccaatcggtatctctgattggaattccattttctggattacccatccaggtggtaaggccatcttggataaggttgaagaaaagttgcatttaaagtctgataagttcgttgactctcgtcacgttttgtctgaacatggtaacatgtcttcttccactgttttgtttgttatggatgaattgagaaaaagatccttggaagaaggtaagtctactactggtgatggttttgaatggggtgtcttgttcggttttggtccaggtttgaccgttgaaagagttgtcgttagatccgttccaatcaagtactaatttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaagattgaaaatctatggaaagatatggacggtagcaacaagaatatagcacgagccgcgaagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctaggtccgccggcgttggacgagcgaaaattcatttaatattcaatgaagttataaattgatagtttaaataaagtcagtctttttcctcatgtttagaattgtattaatgtacgccgtttacgttggagtgtaaatgtgtctattccagaacgaaatctaaatgagcagtacaggcagttcagatggtactgaagcggtactaaagatgcatgaattgaacagatgtggtagttatgtatatgaggaatatgagttgtcacattaaaaatataatagctatgatcccattattatattcgtgacagttcgtaacgttttaattggcttatgtttttgagaaatgggtgaattttaagataattgttgggattccattattgataaaggctataatattaggtatacagaatatactggaagttctcctcgaggatataggaatcctcaaaatggaatctatatttctatttactaatatcacgattattcttcattccgttttatatgtttcattatcctattacattatcaatccttgcatttcagcttcctctaacttcgatgacagctggcggtttaaacgcgtggccgtgccgtc

SEQ ID NO: 4 - DNA, 6787 bp, Artificial Sequence
  source: /note=“Description of Artificial Sequence Synthetic polynucleotide”
gacggcacggccacgcgtttaaaccgccagagtatgtcaactggcgcagtagatacatgtttttctcttccacgtcgaattttgttatatacatagcataatcgagttgtatgcaccctttttgtttatctcgttagtaactcggggtaggaataagacatccacaaaggtgacagaacaaaatcatcctagccttgttcataatctacctctatatagccgctaaaaaattagtagtattttgactctttaagagcacatttattatcaggctgcttttacatacttcttttgtttaaaacatttaaagacgatcactgcccttccaaaggacaaatatatatacacaaacactaggccaaaagttcacttataataatttagtggtaattatgttgggtaaagaaattgccaatagtctttttttttccgtattgtaaggtgagactgaggtagcggcacaaaaaaacgacacataataggatactgagtaaagcagtattaaaataaaaagatatattttacctcgaacgctacaaataaagcagaaaagaacaaaatcgtgagccgctcgtccaacgccggcggacctagctttccaaaaggagaggcaaagcaatttaagaatgtatgaacaaaataaaggggaaaaattaccccctctactttaccaaacgaatactaccaataatatttacaacttttccttatgattttttcactgaagcgcttcgcaatagttgtgagcgatatcaaaagtaacgaaatgaacttcgcggctcgtgctatattcttgttgctaccgtccatatctttccatagattttcaatctttgatgtctccatggtggtacagagaacttgtaaacaattcggtccctacatgtgaggaaattcgctgtgacacttttatcactgaactccaaatttaaaaaatagcataaaattcgttatacagcaaatctatgtgttgcaattaagaactaaaagatatagagtgcatattttcaagaaggatagtaagctggcaaattattcaaaatgggagaattgttgacgcaaaactctacgcatgatcttgttggtggcagttctaggcaaagaagacaaagggacgactctagtaaccttaaacaatggattcaacttcttttgcaaacccaagttgaaggacaatctcaattggttcaagtcgatagtagtatcgttagaatccttcaagacgaagaaaataaccaattgttctggaccaccacctaatggtggaacaccgatagcagtggtttcgaaaactctgtcatcgacttcgttacaaactctttcaatctcaatggaagagattttgataccaccgatgttcatggtgtcatcagcacgaccgtgagcatggtagtaaccgttggaagttaattcaaagatgtcaccgtgtcttctcaaaacttcaccgttcaaagttggcatacctttgaagtagacatcgtggtggttaccgttcaataaagtcttagaagcaccgaacataactggacccaaagccaattcaccaatacctggcttgttctttggcattgggtaaccgttcttatccaaaatgtacaaagtacaacccatacattgggaagaaaaggaggacaaggattgggcttgtaagaaagaaccagcagagaaagcaccaccgatttcggtaccaccacacatttcgataacaggtttatagttggctctacccatcaaccacaagtattcatcgacgttagaagcttcaccagaggaagaaaagcaacggatggtagaccagtcataaccggaaacgcagttggtggatttccaagatctaacaatagatggaacaacacctaacatagtaaccttagcgtcttggacgaacttggcgaaaccagaaaccaatggggaaccattatacaaagcgatagaagcaccgttcaataaagaggcgtaaaccaaccatggacccatcatccaacctaaattagttggccaaacaatgacgtcacctttacgaatatccaagtgagaccaaccgtcggcagcagccttcaatggagtagcttgggtccatggaatggcctttggttcaccagtggtaccggaagagaataaaatgttggtgtaggcatcaactggttgttcacgagcggtgaattcacagttcttgaattccttagcacgttccaagaaataatcccaggaaatgtcaccgtcacgcaattcggcaccgatgttggaaccggaacatggaatgacaatagccattggagacttagcttcaacgactctagaatacaatggaattctcttcttaccacggatgatgtggtcttgagtgaagatggccttagccttagacaatctcaatctagtagagatttctggagcggagaaagaatcagcgatggaaacgacgacgtaaccagccaagacaatggctaagtagatgacgacagcgtcaacgtgcattggcatatcgatggcgatagcacaacctttttccaaacccatttcttccaaggcataaccaaccaaccaaactctctttctcaattggtccaaagtcaacttgttcaatggcaaatcgtcgttaccctcatcacgccaaacgatcatagtatcattcaactttttgttagagttaacattcaagcagttcttagcagagttcaagtaaccacctggcaaccattcggaaccacctgggttgttaatatcgtctctacgtaagatacattctggatctttagaaaaggagatcttcatttcatccattaaaacagttctccagtaaacttctgggtttctgacggagaactcttggaagtgagaaaaagaagaaattggatccttgtatttaacacccaagaattccttacctcttttctccaacaaagcacccaagttggtagacttgaccttttcagggtctggaatccaagctggtggggctggaccaaagtccttgtaacaaccatagaataacatttggtgcaaggaaaatggcaagtctggggataagatatggttggcaatgttaatccaagtttgtggggtagcagcaccgtaattacaaacaatttcagctaatctaccatgcaaagtttcggcgacctcagaggtaatacccaaagcgatgaaatcggaagcaacaacagaatccaaagatttgtagttcttacccatttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactataatgaaccacttaagagctgaaggtccagcttccgttttggccattggtaccgctaacccagaaaacatcttgttgcaagacgaatttccagactactacttcagagtcactaagtccgaacacatgacccaattgaaggaaaagttcagaaagatttgtgataagtctatgatcagaaaaagaaactgtttcttgaacgaagaacacttgaaacaaaaccctagattagttgaacatgaaatgcaaactttagatgccagacaagatatgttggtcgtcgaagtcccaaagttgggtaaggacgcttgtgccaaggctatcaaggaatggggtcaaccaaagtctaagattactcatttgatcttcacttccgcctctaccaccgatatgccaggtgctgattaccattgtgctaagttgttgggtttatccccatctgttaaaagagttatgatgtaccaattgggttgttatggtggtggtactgttttgagaattgccaaagacatcgctgaaaacaataagggtgctagagttttggctgtttgttgtgatattatggcttgtttgttcagaggtccatccgagtctgatttagagttgttagttggtcaagctattttcggtgacggtgctgctgctgttattgttggtgctgaaccagacgaatctgttggtgaacgtccaatctttgaattggtctctaccggtcaaaccatcttgccaaactctgaaggtaccattggtggtcacatcagagaagctggtttgatcttcgatttgcataaagatgttcctatgttgatttctaataacatcgaaaagtgcttaatcgaagctttcactccaatcggtatctctgattggaattccattttctggattacccatccaggtggtaaggccatcttggataaggttgaagaaaagttgcatttaaagtctgataagttcgttgactctcgtcacgttttgtctgaacatggtaacatgtcttcttccactgttttgtttgttatggatgaattgagaaaaagatccttggaagaaggtaagtctactactggtgatggttttgaatggggtgtcttgttcggttttggtccaggtttgaccgttgaaagagttgtcgttagatccgttccaatcaagtactaagtatacttcttttttttactttgttcagaacaacttctcatttttttctactcataactttagcatcacaaagtacacaataataacgagtagtaacacttttatagttcatacatgcttcaactacttaataaatgattgtatgataatgttttcaatgtaagagatttcgattatccacaaactttgaaacacagggacacaattcttgatatgctttcaaccgctgcgttttggatacctattcttgacataatatgactaccattttgttattgtacgtggggcagttgacgtcttatcatatgtcaaagtcatttgcgaagttcttggcaagttgccaactgacgagatgcagtaaaaagagattgccgtcttgaaactttttgtccttttttttttccggggactctacgagaaccctttgtcctactgatccccgcgtgcttggccggccgtgatcatctacccatgccgaaattcgggccgttggccggattgcgcgttgtcttctccggtatcgaaatcgccggaccgtttgccgggcaaatgttcgcagaatggggcgcggaagttatctggatcgagaacgtcgcctgggccgacaccattcgcgttcaaccgaactacccgcaactctcccgccgcaatttgcacgcgctgtcgttaaatattttcaaagatgaaggccgcgaagcgtttctgaaattaatggaaaccaccgatatcttcatcgaagccagtaaaggtccggcctttgcccgtcgtggcattaccgatgaagtactgtggcagcacaacccgaaactggttatcgctcacctgtccggttttggtcagtacggcaccgaggagtacaccaatcttccggcctataacactatcgcccaggcctttagtggttacctgattcagaacggtgatgttgaccagccaatgcctgccttcccgtataccgccgattacttttctggcctgaccgccaccacggcggcgctggcagcactgcataaagtgcgtgaaaccggtaaaggcgaaagtatcgacatcgccatgtatgaagtgatgctgcgtatgggccagtacttcatgatggattacttcaacggcggcgaaatgtgcccgcgcatgagcaaaggtaaagatccctactacgccgaggtccgccggcgttggacgagcgactttaatgtcgttctccctttttaaagagtaaatacatatttaaaaaagtgactatggctattgctaaacgtgataaaaatcagagcctataacactctctgaaataacgctatgcaggaatttccagttaagttcttcttggggtgacttctttactcggtatgatatgtgttttatatgcacagtacgagtccattagggtaaattagtggccgagaaacttttgccgccgagcttttaagtatccttttgccacttcttatttagataaagacctggcagtagtagtcgtagaagataagatagacagagaatgaatactaataagatagcacaagacgaagtccaagataaggttttgcaaagagcagaactagcacattctgtatggaacttaaggttcaacctcagtaaagttgccaaacggattcgcatggaaacaaaggtatttccagagataaagataaatgacgcgcaatcacagttagagcgatctaggtgtagaatatttagccctgacctggaggaagaacatgtgcccttgattcaaggcggcggtttaaacgcgtggccgtgccgtc

SEQ ID NO: 5 - DNA, 8966 bp, Artificial Sequence
  source: /note=“Description of Artificial Sequence Synthetic polynucleotide”
gacggcacggccacgcgtttaaaccgccagagtatgtcaactggcgcagtagatacatgtttttctcttccacgtcgaattttgttatatacatagcataatcgagttgtatgcaccctttttgtttatctcgttagtaactcggggtaggaataagacatccacaaaggtgacagaacaaaatcatcctagccttgttcataatctacctctatatagccgctaaaaaattagtagtattttgactctttaagagcacatttattatcaggctgcttttacatacttcttttgtttaaaacatttaaagacgatcactgcccttccaaaggacaaatatatatacacaaacactaggccaaaagttcacttataataatttagtggtaattatgttgggtaaagaaattgccaatagtctttttttttccgtattgtaaggtgagactgaggtagcggcacaaaaaaacgacacataataggatactgagtaaagcagtattaaaataaaaagatatattttacctcgaacgctacaaataaagcagaaaagaacaaaatcgtgagccgctcgtccaacgccggcggacctagctttccaaaaggagaggcaaagcaatttaagaatgtatgaacaaaataaaggggaaaaattaccccctctactttaccaaacgaatactaccaataatatttacaacttttccttatgattttttcactgaagcgcttcgcaatagttgtgagcgatatcaaaagtaacgaaatgaacttcgcggctcgtgctatattcttgttgctaccgtccatatctttccatagattttcaatctttgatgtctccatggtggtacagagaacttgtaaacaattcggtccctacatgtgaggaaattcgctgtgacacttttatcactgaactccaaatttaaaaaatagcataaaattcgttatacagcaaatctatgtgttgcaattaagaactaaaagatatagagtgcatattttcaagaaggatagtaagctggcaaattattcaaaatgggagaattgttgacgcaaaactctacgcatgatcttgttggtggcagttctaggcaaagaagacaaagggacgactctagtaaccttaaacaatggattcaacttcttttgcaaacccaagttgaaggacaatctcaattggttcaagtcgatagtagtatcgttagaatccttcaagacgaagaaaataaccaattgttctggaccaccacctaatggtggaacaccgatagcagtggtttcgaaaactctgtcatcgacttcgttacaaactctttcaatctcaatggaagagattttgataccaccgatgttcatggtgtcatcagcacgaccgtgagcatggtagtaaccgttggaagttaattcaaagatgtcaccgtgtcttctcaaaacttcaccgttcaaagttggcatacctttgaagtagacatcgtggtggttaccgttcaataaagtcttagaagcaccgaacataactggacccaaagccaattcaccaatacctggcttgttctttggcattgggtaaccgttcttatccaaaatgtacaaagtacaacccatacattgggaagaaaaggaggacaaggattgggcttgtaagaaagaaccagcagagaaagcaccaccgatttcggtaccaccacacatttcgataacaggtttatagttggctctacccatcaaccacaagtattcatcgacgttagaagcttcaccagaggaagaaaagcaacggatggtagaccagtcataaccggaaacgcagttggtggatttccaagatctaacaatagatggaacaacacctaacatagtaaccttagcgtcttggacgaacttggcgaaaccagaaaccaatggggaaccattatacaaagcgatagaagcaccgttcaataaagaggcgtaaaccaaccatggacccatcatccaacctaaattagttggccaaacaatgacgtcacctttacgaatatccaagtgagaccaaccgtcggcagcagccttcaatggagtagcttgggtccatggaatggcctttggttcaccagtggtaccggaagagaataaaatgttggtgtaggcatcaactggttgttcacgagcggtgaattcacagttcttgaattccttagcacgttccaagaaataatcccaggaaatgtcaccgtcacgcaattcggcaccgatgttggaaccggaacatggaatgacaatagccattggagacttagcttcaacgactctagaatacaatggaattctcttcttaccacggatgatgtggtcttgagtgaagatggccttagccttagacaatctcaatctagtagagatttctggagcggagaaagaatcagcgatggaaacgacgacgtaaccagccaagacaatggctaagtagatgacgacagcgtcaacgtgcattggcatatcgatggcgatagcacaacctttttccaaacccatttcttccaaggcataaccaaccaaccaaactctctttctcaattggtccaaagtcaacttgttcaatggcaaatcgtcgttaccctcatcacgccaaacgatcatagtatcattcaactttttgttagagttaacattcaagcagttcttagcagagttcaagtaaccacctggcaaccattcggaaccacctgggttgttaatatcgtctctacgtaagatacattctggatctttagaaaaggagatcttcatttcatccattaaaacagttctccagtaaacttctgggtttctgacggagaactcttggaagtgagaaaaagaagaaattggatccttgtatttaacacccaagaattccttacctcttttctccaacaaagcacccaagttggtagacttgaccttttcagggtctggaatccaagctggtggggctggaccaaagtccttgtaacaaccatagaataacatttggtgcaaggaaaatggcaagtctggggataagatatggttggcaatgttaatccaagtttgtggggtagcagcaccgtaattacaaacaatttcagctaatctaccatgcaaagtttcggcgacctcagaggtaatacccaaagcgatgaaatcggaagcaacaacagaatccaaagatttgtagttcttacccatttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactataatgaaccacttaagagctgaaggtccagcttccgttttggccattggtaccgctaacccagaaaacatcttgttgcaagacgaatttccagactactacttcagagtcactaagtccgaacacatgacccaattgaaggaaaagttcagaaagatttgtgataagtctatgatcagaaaaagaaactgtttcttgaacgaagaacacttgaaacaaaaccctagattagttgaacatgaaatgcaaactttagatgccagacaagatatgttggtcgtcgaagtcccaaagttgggtaaggacgcttgtgccaaggctatcaaggaatggggtcaaccaaagtctaagattactcatttgatcttcacttccgcctctaccaccgatatgccaggtgctgattaccattgtgctaagttgttgggtttatccccatctgttaaaagagttatgatgtaccaattgggttgttatggtggtggtactgttttgagaattgccaaagacatcgctgaaaacaataagggtgctagagttttggctgtttgttgtgatattatggcttgtttgttcagaggtccatccgagtctgatttagagttgttagttggtcaagctattttcggtgacggtgctgctgctgttattgttggtgctgaaccagacgaatctgttggtgaacgtccaatctttgaattggtctctaccggtcaaaccatcttgccaaactctgaaggtaccattggtggtcacatcagagaagctggtttgatcttcgatttgcataaagatgttcctatgttgatttctaataacatcgaaaagtgcttaatcgaagctttcactccaatcggtatctctgattggaattccattttctggattacccatccaggtggtaaggccatcttggataaggttgaagaaaagttgcatttaaagtctgataagttcgttgactctcgtcacgttttgtctgaacatggtaacatgtcttcttccactgttttgtttgttatggatgaattgagaaaaagatccttggaagaaggtaagtctactactggtgatggttttgaatggggtgtcttgttcggttttggtccaggtttgaccgttgaaagagttgtcgttagatccgttccaatcaagtactaagtatacttcttttttttactttgttcagaacaacttctcatttttttctactcataactttagcatcacaaagtacacaataataacgagtagtaacacttttatagttcatacatgcttcaactacttaataaatgattgtatgataatgttttcaatgtaagagatttcgattatccacaaactttgaaacacagggacacaattcttgatatgctttcaaccgctgcgttttggatacctattcttgacataatatgactaccattttgttattgtacgtggggcagttgacgtcttatcatatgtcaaagtcatttgcgaagttcttggcaagttgccaactgacgagatgcagtaaaaagagattgccgtcttgaaactttttgtccttttttttttccggggactctacgagaaccctttgtcctactgatccccgcgtgcttggccggccgtgatcatctacccatgccgaaattcgggccgttggccggattgcgcgttgtcttctccggtatcgaaatcgccggaccgtttgccgggcaaatgttcgcagaatggggcgcggaagttatctggatcgagaacgtcgcctgggccgacaccattcgcgttcaaccgaactacccgcaactctcccgccgcaatttgcacgcgctgtcgttaaatattttcaaagatgaaggccgcgaagcgtttctgaaattaatggaaaccaccgatatcttcatcgaagccagtaaaggtccggcctttgcccgtcgtggcattaccgatgaagtactgtggcagcacaacccgaaactggttatcgctcacctgtccggttttggtcagtacggcaccgaggagtacaccaatcttccggcctataacactatcgcccaggcctttagtggttacctgattcagaacggtgatgttgaccagccaatgcctgccttcccgtataccgccgattacttttctggcctgaccgccaccacggcggcgctggcagcactgcataaagtgcgtgaaaccggtaaaggcgaaagtatcgacatcgccatgtatgaagtgatgctgcgtatgggccagtacttcatgatggattacttcaacggcggcgaaatgtgcccgcgcatgagcaaaggtaaagatccctactacgccgacggccggccaagcacgcggggatcagtaggacaaagggttctcgtagagtccccggaaaaaaaaaaggacaaaaagtttcaagacggcaatctctttttactgcatctcgtcagttggcaacttgccaagaacttcgcaaatgactttgacatatgataagacgtcaactgccccacgtacaataacaaaatggtagtcatattatgtcaagaataggtatccaaaacgcagcggttgaaagcatatcaagaattgtgtccctgtgtttcaaagtttgtggataatcgaaatctcttacattgaaaacattatcatacaatcatttattaagtagttgaagcatgtatgaactataaaagtgttactactcgttattattgtgtactttgtgatgctaaagttatgagtagaaaaaaatgagaagttgttctgaacaaagtaaaaaaaagaagtatacttactttctaggggtgtaatcaaagatcaacaacttttcccagaaagatctgtaaacgtcaccgaaaccaacatgagctgggtgaataatgtagtcttggatagtttcaacagattcgaaggtgacttcaacaatatgagtgtaaccttcttctttgttcttttgggtgacgtctttaccccagtagacatccttcatagctggaataatgttaaccaagttaacgtaagttttgaagaattcctctttttgggcttcggtaatttcgtctttgaactttaagacgatcaagtgtttaacagccattatagttttttctccttgacgttaaagtatagaggtatattaacaattttttgttgatacttttatgacatttgaataagaagtaatacaaaccgaaaatgttgaaagtattagttaaagtggttatgcagcttttgcatttatatatctgttaatagatcaaaaatcatcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataaatggctgttaaacacttgatcgtcttaaagttcaaagacgaaattaccgaagcccaaaaagaggaattcttcaaaacttacgttaacttggttaacattattccagctatgaaggatgtctactggggtaaagacgtcacccaaaagaacaaagaagaaggttacactcatattgttgaagtcaccttcgaatctgttgaaactatccaagactacattattcacccagctcatgttggtttcggtgacgtttacagatctttctgggaaaagttgttgatctttgattacacccctagaaagtaatttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaagattgaaaatctatggaaagatatggacggtagcaacaagaatatagcacgagccgcgaagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctaggtccgccggcgttggacgagcgactttaatgtcgttctccctttttaaagagtaaatacatatttaaaaaagtgactatggctattgctaaacgtgataaaaatcagagcctataacactctctgaaataacgctatgcaggaatttccagttaagttcttcttggggtgacttctttactcggtatgatatgtgttttatatgcacagtacgagtccattagggtaaattagtggccgagaaacttttgccgccgagcttttaagtatccttttgccacttcttatttagataaagacctggcagtagtagtcgtagaagataagatagacagagaatgaatactaataagatagcacaagacgaagtccaagataaggttttgcaaagagcagaactagcacattctgtatggaacttaaggttcaacctcagtaaagttgccaaacggattcgcatggaaacaaaggtatttccagagataaagataaatgacgcgcaatcacagttagagcgatctaggtgtagaatatttagccctgacctggaggaagaacatgtgcccttgattcaaggcggcggtttaaacgcgtggccgtgccgtc

SEQ ID NO: 6 - DNA, 8921 bp, Artificial Sequence
  source: /note=“Description of Artificial Sequence Synthetic polynucleotide”
gacggcacggccacgcgtttaaaccgcctacgccatcattaaagacctggtcaactataaaataatacaatcaatacttgcttgaacgcttgattttactgatattctatccaaaagcaagtagaccagaaactctcaagatgttgcaaataccgttcgatgtttttggtttagattgttttaatgttgatgcttttttacttatttttggaagcgtctttttaatttagttttatattataggtatatgaatgtgtttatgccaataagggtttttttgtacagttatgtgattataaacagtcttttgtctagtttttttcaccagtatcggcctctatttataaaaaacggagcagctttcggtgtcagtaattctgaaaaaatttgtgtcactctgattgtaaatgaattaatttagctagatagttgcgagccccaacgagaagattgtcagacaaagacaacattcaacaacctacatccgttactattcgttaactcgaggtacttgaaacttttcagttaagtcgctcgtccaacgccggcggacctagctttccaaaaggagaggcaaagcaatttaagaatgtatgaacaaaataaaggggaaaaattaccccctctactttaccaaacgaatactaccaataatatttacaacttttccttatgattttttcactgaagcgcttcgcaatagttgtgagcgatatcaaaagtaacgaaatgaacttcgcggctcgtgctatattcttgttgctaccgtccatatctttccatagattttcaatctttgatgtctccatggtggtacagagaacttgtaaacaattcggtccctacatgtgaggaaattcgctgtgacacttttatcactgaactccaaatttaaaaaatagcataaaattcgttatacagcaaatctatgtgttgcaattaagaactaaaagatatagagtgcatattttcaagaaggatagtaagctggcaaattactttctaggggtgtaatcaaagatcaacaacttttcccagaaagatctgtaaacgtcaccgaaaccaacatgagctgggtgaataatgtagtcttggatagtttcaacagattcgaaggtgacttcaacaatatgagtgtaaccttcttctttgttcttttgggtgacgtctttaccccagtagacatccttcatagctggaataatgttaaccaagttaacgtaagttttgaagaattcctctttttgggcttcggtaatttcgtctttgaactttaagacgatcaagtgtttaacagccatttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactataatggctgttaaacacttgatcgtcttaaagttcaaagacgaaattaccgaagcccaaaaagaggaattcttcaaaacttacgttaacttggttaacattattccagctatgaaggatgtctactggggtaaagacgtcacccaaaagaacaaagaagaaggttacactcatattgttgaagtcaccttcgaatctgttgaaactatccaagactacattattcacccagctcatgttggtttcggtgacgtttacagatctttctgggaaaagttgttgatctttgattacacccctagaaagtaagtatacttcttttttttactttgttcagaacaacttctcatttttttctactcataactttagcatcacaaagtacacaataataacgagtagtaacacttttatagttcatacatgcttcaactacttaataaatgattgtatgataatgttttcaatgtaagagatttcgattatccacaaactttgaaacacagggacacaattcttgatatgctttcaaccgctgcgttttggatacctattcttgacataatatgactaccattttgttattgtacgtggggcagttgacgtcttatcatatgtcaaagtcatttgcgaagttcttggcaagttgccaactgacgagatgcagtaaaaagagattgccgtcttgaaactttttgtccttttttttttccggggactctacgagaaccctttgtcctactgatccccgcgtgcttggccggccgtgcgagtaagcaactctggcgctggcatggcataaccggcgacggcaatgcgcaagatgggatgctatgggcagagagccgtactttactgcttatggcactacagcaacagatggttaccccactaagcctgaagcgaatcgccatcaattctgcgcagtggcgaggagataaaagcgcggaagtcattcatcaactggcgacgctactcaaagcagggttaacgctttctgaagggctggctctgctggcggaacagcatcccagtaagcaatggcaagcgttgctgcaatcgctggcgcacgatctcgaacagggcattgctttttccaatgccttattaccctggtcagaggtatttccgccgctctatcaggcgatgatccgcacgggtgaactgaccggtaagctggatgaatgctgctttgaactggcgcgtcagcaaaaagcccagcgtcagttgaccgacaaagtgaaatcagcgttacgttatcccatcatcattttagcgatggcaatcatggtggttgtggcaatgctgcattttgttctgccggagtttgccgctatctataagaccttcaacaccccactaccggcactaacgcaggggatcatgacgctggcagactttagtggcgaatggagctggctgctggtgttgttcggctttctgctggcgatagccaataagttgctgaacggccggccaagcacgcggggatcagtaggacaaagggttctcgtagagtccccggaaaaaaaaaaggacaaaaagtttcaagacggcaatctctttttactgcatctcgtcagttggcaacttgccaagaacttcgcaaatgactttgacatatgataagacgtcaactgccccacgtacaataacaaaatggtagtcatattatgtcaagaataggtatccaaaacgcagcggttgaaagcatatcaagaattgtgtccctgtgtttcaaagtttgtggataatcgaaatctcttacattgaaaacattatcatacaatcatttattaagtagttgaagcatgtatgaactataaaagtgttactactcgttattattgtgtactttgtgatgctaaagttatgagtagaaaaaaatgagaagttgttctgaacaaagtaaaaaaaagaagtatacttattcaaaatgggagaattgttgacgcaaaactctacgcatgatcttgttggtggcagttctaggcaaagaagacaaagggacgactctagtaaccttaaacaatggattcaacttcttttgcaaacccaagttgaaggacaatctcaattggttcaagtcgatagtagtatcgttagaatccttcaagacgaagaaaataaccaattgttctggaccaccacctaatggtggaacaccgatagcagtggtttcgaaaactctgtcatcgacttcgttacaaactctttcaatctcaatggaagagattttgataccaccgatgttcatggtgtcatcagcacgaccgtgagcatggtagtaaccgttggaagttaattcaaagatgtcaccgtgtcttctcaaaacttcaccgttcaaagttggcatacctttgaagtagacatcgtggtggttaccgttcaataaagtcttagaagcaccgaacataactggacccaaagccaattcaccaatacctggcttgttctttggcattgggtaaccgttcttatccaaaatgtacaaagtacaacccatacattgggaagaaaaggaggacaaggattgggcttgtaagaaagaaccagcagagaaagcaccaccgatttcggtaccaccacacatttcgataacaggtttatagttggctctacccatcaaccacaagtattcatcgacgttagaagcttcaccagaggaagaaaagcaacggatggtagaccagtcataaccggaaacgcagttggtggatttccaagatctaacaatagatggaacaacacctaacatagtaaccttagcgtcttggacgaacttggcgaaaccagaaaccaatggggaaccattatacaaagcgatagaagcaccgttcaataaagaggcgtaaaccaaccatggacccatcatccaacctaaattagttggccaaacaatgacgtcacctttacgaatatccaagtgagaccaaccgtcggcagcagccttcaatggagtagcttgggtccatggaatggcctttggttcaccagtggtaccggaagagaataaaatgttggtgtaggcatcaactggttgttcacgagcggtgaattcacagttcttgaattccttagcacgttccaagaaataatcccaggaaatgtcaccgtcacgcaattcggcaccgatgttggaaccggaacatggaatgacaatagccattggagacttagcttcaacgactctagaatacaatggaattctcttcttaccacggatgatgtggtcttgagtgaagatggccttagccttagacaatctcaatctagtagagatttctggagcggagaaagaatcagcgatggaaacgacgacgtaaccagccaagacaatggctaagtagatgacgacagcgtcaacgtgcattggcatatcgatggcgatagcacaacctttttccaaacccatttcttccaaggcataaccaaccaaccaaactctctttctcaattggtccaaagtcaacttgttcaatggcaaatcgtcgttaccctcatcacgccaaacgatcatagtatcattcaactttttgttagagttaacattcaagcagttcttagcagagttcaagtaaccacctggcaaccattcggaaccacctgggttgttaatatcgtctctacgtaagatacattctggatctttagaaaaggagatcttcatttcatccattaaaacagttctccagtaaacttctgggtttctgacggagaactcttggaagtgagaaaaagaagaaattggatccttgtatttaacacccaagaattccttacctcttttctccaacaaagcacccaagttggtagacttgaccttttcagggtctggaatccaagctggtggggctggaccaaagtccttgtaacaaccatagaataacatttggtgcaaggaaaatggcaagtctggggataagatatggttggcaatgttaatccaagtttgtggggtagcagcaccgtaattacaaacaatttcagctaatctaccatgcaaagtttcggcgacctcagaggtaatacccaaagcgatgaaatcggaagcaacaacagaatccaaagatttgtagttcttacccattatagttttttctccttgacgttaaagtatagaggtatattaacaattttttgttgatacttttatgacatttgaataagaagtaatacaaaccgaaaatgttgaaagtattagttaaagtggttatgcagcttttgcatttatatatctgttaatagatcaaaaatcatcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataaatgaaccacttaagagctgaaggtccagcttccgttttggccattggtaccgctaacccagaaaacatcttgttgcaagacgaatttccagactactacttcagagtcactaagtccgaacacatgacccaattgaaggaaaagttcagaaagatttgtgataagtctatgatcagaaaaagaaactgtttcttgaacgaagaacacttgaaacaaaaccctagattagttgaacatgaaatgcaaactttagatgccagacaagatatgttggtcgtcgaagtcccaaagttgggtaaggacgcttgtgccaaggctatcaaggaatggggtcaaccaaagtctaagattactcatttgatcttcacttccgcctctaccaccgatatgccaggtgctgattaccattgtgctaagttgttgggtttatccccatctgttaaaagagttatgatgtaccaattgggttgttatggtggtggtactgttttgagaattgccaaagacatcgctgaaaacaataagggtgctagagttttggctgtttgttgtgatattatggcttgtttgttcagaggtccatccgagtctgatttagagttgttagttggtcaagctattttcggtgacggtgctgctgctgttattgttggtgctgaaccagacgaatctgttggtgaacgtccaatctttgaattggtctctaccggtcaaaccatcttgccaaactctgaaggtaccattggtggtcacatcagagaagctggtttgatcttcgatttgcataaagatgttcctatgttgatttctaataacatcgaaaagtgcttaatcgaagctttcactccaatcggtatctctgattggaattccattttctggattacccatccaggtggtaaggccatcttggataaggttgaagaaaagttgcatttaaagtctgataagttcgttgactctcgtcacgttttgtctgaacatggtaacatgtcttcttccactgttttgtttgttatggatgaattgagaaaaagatccttggaagaaggtaagtctactactggtgatggttttgaatggggtgtcttgttcggttttggtccaggtttgaccgttgaaagagttgtcgttagatccgttccaatcaagtactaatttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaagattgaaaatctatggaaagatatggacggtagcaacaagaatatagcacgagccgcgaagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctaggtccgccggcgttggacgagcgaaaattcatttaatattcaatgaagttataaattgatagtttaaataaagtcagtctttttcctcatgtttagaattgtattaatgtacgccgtttacgttggagtgtaaatgtgtctattccagaacgaaatctaaatgagcagtacaggcagttcagatggtactgaagcggtactaaagatgcatgaattgaacagatgtggtagttatgtatatgaggaatatgagttgtcacattaaaaatataatagctatgatcccattattatattcgtgacagttcgtaacgttttaattggcttatgtttttgagaaatgggtgaattttaagataattgttgggattccattattgataaaggctataatattaggtatacagaatatactggaagttctcctcgaggatataggaatcctcaaaatggaatctatatttctatttactaatatcacgattattcttcattccgttttatatgtttcattatcctattacattatcaatccttgcatttcagcttcctctaacttcgatgacagctggcggtttaaacgcgtggccgtgccgtc

SEQ ID NO: 7 - DNA, 2163 bp, Artificial Sequence
  source: /note=“Description of Artificial Sequence Synthetic polynucleotide”
atgggtaagaactacaaatctttggattctgttgttgcttccgatttcatcgctttgggtattacctctgaggtcgccgaaactttgcatggtagattagctgaaattgtttgtaattacggtgctgctaccccacaaacttggattaacattgccaaccatatcttatccccagacttgccattttccttgcaccaaatgttattctatggttgttacaaggactttggtccagccccaccagcttggattccagaccctgaaaaggtcaagtctaccaacttgggtgctttgttggagaaaagaggtaaggaattcttgggtgttaaatacaaggatccaatttcttctttttctcacttccaagagttctccgtcagaaacccagaagtttactggagaactgttttaatggatgaaatgaagatctccttttctaaagatccagaatgtatcttacgtagagacgatattaacaacccaggtggttccgaatggttgccaggtggttacttgaactctgctaagaactgcttgaatgttaactctaacaaaaagttgaatgatactatgatcgtttggcgtgatgagggtaacgacgatttgccattgaacaagttgactttggaccaattgagaaagagagtttggttggttggttatgccttggaagaaatgggtttggaaaaaggttgtgctatcgccatcgatatgccaatgcacgttgacgctgtcgtcatctacttagccattgtcttggctggttacgtcgtcgtttccatcgctgattctttctccgctccagaaatctctactagattgagattgtctaaggctaaggccatcttcactcaagaccacatcatccgtggtaagaagagaattccattgtattctagagtcgttgaagctaagtctccaatggctattgtcattccatgttccggttccaacatcggtgccgaattgcgtgacggtgacatttcctgggattatttcttggaacgtgctaaggaattcaagaactgtgaattcaccgctcgtgaacaaccagttgatgcctacaccaacattttattctcttccggtaccactggtgaaccaaaggccattccatggacccaagctactccattgaaggctgctgccgacggttggtctcacttggatattcgtaaaggtgacgtcattgtttggccaactaatttaggttggatgatgggtccatggttggtttacgcctctttattgaacggtgcttctatcgctttgtataatggttccccattggtttctggtttcgccaagttcgtccaagacgctaaggttactatgttaggtgttgttccatctattgttagatcttggaaatccaccaactgcgtttccggttatgactggtctaccatccgttgcttttcttcctctggtgaagcttctaacgtcgatgaatacttgtggttgatgggtagagccaactataaacctgttatcgaaatgtgtggtggtaccgaaatcggtggtgctttctctgctggttctttcttacaagcccaatccttgtcctccttttcttcccaatgtatgggttgtactttgtacattttggataagaacggttacccaatgccaaagaacaagccaggtattggtgaattggctttgggtccagttatgttcggtgcttctaagactttattgaacggtaaccaccacgatgtctacttcaaaggtatgccaactttgaacggtgaagttttgagaagacacggtgacatctttgaattaacttccaacggttactaccatgctcacggtcgtgctgatgacaccatgaacatcggtggtatcaaaatctcttccattgagattgaaagagtttgtaacgaagtcgatgacagagttttcgaaaccactgctatcggtgttccaccattaggtggtggtccagaacaattggttattttcttcgtcttgaaggattctaacgatactactatcgacttgaaccaattgagattgtccttcaacttgggtttgcaaaagaagttgaatccattgtttaaggttactagagtcgtccctttgtcttctttgcctagaactgccaccaacaagatcatgcgtagagttttgcgtcaacaattctcccattttgaataa

SEQ ID NO: 8 - DNA, 1158 bp, Artificial Sequence
  source: /note=“Description of Artificial Sequence Synthetic polynucleotide”
atgaaccacttaagagctgaaggtccagcttccgttttggccattggtaccgctaacccagaaaacatcttgttgcaagacgaatttccagactactacttcagagtcactaagtccgaacacatgacccaattgaaggaaaagttcagaaagatttgtgataagtctatgatcagaaaaagaaactgtttcttgaacgaagaacacttgaaacaaaaccctagattagttgaacatgaaatgcaaactttagatgccagacaagatatgttggtcgtcgaagtcccaaagttgggtaaggacgcttgtgccaaggctatcaaggaatggggtcaaccaaagtctaagattactcatttgatcttcacttccgcctctaccaccgatatgccaggtgctgattaccattgtgctaagttgttgggtttatccccatctgttaaaagagttatgatgtaccaattgggttgttatggtggtggtactgttttgagaattgccaaagacatcgctgaaaacaataagggtgctagagttttggctgtttgttgtgatattatggcttgtttgttcagaggtccatccgagtctgatttagagttgttagttggtcaagctattttcggtgacggtgctgctgctgttattgttggtgctgaaccagacgaatctgttggtgaacgtccaatctttgaattggtctctaccggtcaaaccatcttgccaaactctgaaggtaccattggtggtcacatcagagaagctggtttgatcttcgatttgcataaagatgttcctatgttgatttctaataacatcgaaaagtgcttaatcgaagctttcactccaatcggtatctctgattggaattccattttctggattacccatccaggtggtaaggccatcttggataaggttgaagaaaagttgcatttaaagtctgataagttcgttgactctcgtcacgttttgtctgaacatggtaacatgtcttcttccactgttttgtttgttatggatgaattgagaaaaagatccttggaagaaggtaagtctactactggtgatggttttgaatggggtgtcttgttcggttttggtccaggtttgaccgttgaaagagttgtcgttagatccgttccaatcaagtactaa

SEQ ID NO: 9 - DNA, 306 bp, Artificial Sequence
  source: /note=“Description of Artificial Sequence Synthetic polynucleotide”
atggctgttaaacacttgatcgtcttaaagttcaaagacgaaattaccgaagcccaaaaagaggaattcttcaaaacttacgttaacttggttaacattattccagctatgaaggatgtctactggggtaaagacgtcacccaaaagaacaaagaagaaggttacactcatattgttgaagtcaccttcgaatctgttgaaactatccaagactacattattcacccagctcatgttggtttcggtgacgtttacagatctttctgggaaaagttgttgatctttgattacacccctagaaagtaa

SEQ ID NO: 10 - protein, 719 aa, Artificial Sequence
  source: /note=“Description of Artificial Sequence Synthetic polypeptide”
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQFSHFE

SEQ ID NO: 11 - protein, 385 aa, Artificial Sequence
  source: /note=“Description of Artificial Sequence Synthetic polypeptide”
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 12 - protein, 101 aa, Artificial Sequence
  source: /note=“Description of Artificial Sequence Synthetic polypeptide”
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 13 - DNA, 500 bp, Artificial Sequence
  source: /note=“Description of Artificial Sequence Synthetic polynucleotide”
tggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactata

SEQ ID NO: 14 - DNA, 501 bp, Artificial Sequence
  source: /note=“Description of Artificial Sequence Synthetic polynucleotide”
catcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataa

SEQ ID NO: 15 - DNA, 509 bp, Artificial Sequence
  source: /note=“Description of Artificial Sequence Synthetic polynucleotide”
ggcttaagtaggttgcaatttctttttctattagtagctaaaaatgggtcacgtgatctatattcgaaaggggcggttgcctcaggaaggcaccggcggtctttcgtccgtgcggagatatctgcgccgttcaggggtccatgtgccttggacgatattaaggcagaaggcagtatcggggcggatcactccgaaccgagattagttaagcccttcccatctcaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacataataaaaaaaataattctttcata

SEQ ID NO: 16 - DNA, 498 bp, Artificial Sequence
  source: /note=“Description of Artificial Sequence Synthetic polynucleotide”
ttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattcttgaaagaatgaaatcgccatgccaagccatcacacggtcttttatgcaattgattgaccgcctgcaacacataggcagtaaaatttttactgaaacgtatataatcatcataagcgacaagtgaggcaacacctttgttaccacattgacaaccccaggtattcatacttcctattagcggaatcaggagtgcaaaaagagaaaataaaagtaaaaaggtagggcaacacatagt

SEQ ID NO: 17 - DNA, 501 bp, Artificial Sequence
  source: /note=“Description of Artificial Sequence Synthetic polynucleotide”
ggacggtagcaacaagaatatagcacgagccgcgaagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaa

SEQ ID NO: 18 - DNA, 489 bp, Artificial Sequence
  source: /note=“Description of Artificial Sequence Synthetic polynucleotide”
gcgacacagagatgacagacggtggcgcaggatccggtttaaacgaggatcccttaagtttaaacaacaacagcaagcaggtgtgcaagacactagagactcctaacatgatgtatgccaataaaacacaagagataaacaacattgcatggaggccccagaggggcgattggtttgggtgcgtgagcggcaagaagtttcaaaacgtccgcgtcctttgagacagcattcgcccagtattttttttattctacaaaccttctataatttcaaagtatttacataattctgtatcagtttaatcaccataatatcgttttctttgtttagtgcaattaatttttcctattgttacttcgggcctttttctgttttatgagctattttttccgtcatccttccccagattttcagcttcatctccagattgtgtctacgtaatgcacgccatcattttaagagaggacagagaagcaagcctcctgaaag

SEQ ID NO: 19 - DNA, 1253 bp, Artificial Sequence
  source: /note=“Description of Artificial Sequence Synthetic polynucleotide”
gatgatggacactagtgtgtcgagaatgtatcaactatatatagtcctaatgccacacaaatatgaagtgggggaagcccattcttaatccggctcaattttggtgcgtgatcgcggcctatgtttgcttccagaaaaagcttagaataatatttctcacctttgatggaatgctcgcgagtgctcgttttgattaccccatatgcattgttgcagcatgcaagcactattgcaagccacgcatggaagaaatttgcaaacacctatagccccgcgttgttgaggaggtggacttggtgtaggaccataaagctgtgcactactatggtgagctctgtcgtctggtgaccttctatctcaggcacatcctcgtttttgtgcatgaggttcgagtcacgcccacggcctattaatccgcgaaataaatgcgaaatctaaattatgacgcaaggctgagagattctgacacgccgcatttgcggggcagtaattatcgggcagttttccggggttcgggatggggtttggagagaaagttcaacacagaccaaaacagcttgggaccacttggatggaggtccccgcagaagagctctggcgcgttggacaaacattgacaatccacggcaaaattgtctacagttccgtgtatgcggatagggatatcttcgggagtatcgcaataggatacaggcactgtgcagattacgcgacatgatagctttgtatgttctacagactctgccgtagcagtctagatataatatcggagttttgtagcgtcgtaaggaaaacttgggttacacaggtttcttgagagccctttgacgttgattgctctggcttccatccaggccctcatgtggttcaggtgcctccgcagtggctggcaagcgtgggggtcaattacgtcacttctattcatgtaccccagactcaattgttgacagcaatttcagcgagaattaaattccacaatcaattctcgctgaaataattaggccgtgatttaattctcgctgaaacagaatcctgtctggggtacagataacaatcaagtaactattatggacgtgcataggaggtggagtccatgacgcaaagggaaatattcattttatcctcgcgaagttgggatgtgtcaaagcgtcgcgctcgctatagtgatgagaatgtctttagtaagcttaagccatataaagaccttccgcctccatatttttttttatccctcttgacaatattaattcctt

SEQ ID NO: 20 - DNA, 1253 bp, Artificial Sequence
  source: /note=“Description of Artificial Sequence Synthetic polynucleotide”
aaggaattaatattgtcaagagggataaaaaaaaatatggaggcggaaggtctttatatggcttaagcttactaaagacattctcatcactatagcgagcgcgacgctttgacacatcccaacttcgcgaggataaaatgaatatttccctttgcgtcatggactccacctcctatgcacgtccataatagttacttgattgttatctgtaccccagacaggattctgtttcagcgagaattaaatcacggcctaattatttcagcgagaattgattgtggaatttaattctcgctgaaattgctgtcaacaattgagtctggggtacatgaatagaagtgacgtaattgacccccacgcttgccagccactgcggaggcacctgaaccacatgagggcctggatggaagccagagcaatcaacgtcaaagggctctcaagaaacctgtgtaacccaagttttccttacgacgctacaaaactccgatattatatctagactgctacggcagagtctgtagaacatacaaagctatcatgtcgcgtaatctgcacagtgcctgtatcctattgcgatactcccgaagatatccctatccgcatacacggaactgtagacaattttgccgtggattgtcaatgtttgtccaacgcgccagagctcttctgcggggacctccatccaagtggtcccaagctgttttggtctgtgttgaactttctctccaaaccccatcccgaaccccggaaaactgcccgataattactgccccgcaaatgcggcgtgtcagaatctctcagccttgcgtcataatttagatttcgcatttatttcgcggattaataggccgtgggcgtgactcgaacctcatgcacaaaaacgaggatgtgcctgagatagaaggtcaccagacgacagagctcaccatagtagtgcacagctttatggtcctacaccaagtccacctcctcaacaacgcggggctataggtgtttgcaaatttcttccatgcgtggcttgcaatagtgcttgcatgctgcaacaatgcatatggggtaatcaaaacgagcactcgcgagcattccatcaaaggtgagaaatattattctaagctttttctggaagcaaacataggccgcgatcacgcaccaaaattgagccggattaagaatgggcttcccccacttcatatttgtgtggcattaggactatatatagttgatacattctcgacacactagtgtccatcatc

SEQ ID NO: 21 - DNA, 665 bp, Artificial Sequence
  source: /note=“Description of Artificial Sequence Synthetic polynucleotide”
gcgcctcaagaaaatgatgctgcaagaagaattgaggaaggaactattcatcttacgttgtttgtatcatcccacgatccaaatcatgttacctacgttaggtacgctaggaactaaaaaaagaaaagaaaagtatgcgttatcactcttcgagccaattcttaattgtgtggggtccgcgaaaatttccggataaatcctgtaaactttaacttaaaccccgtgtttagcgaaattttcaacgaagcgcgcaataaggagaaatattatctaaaagcgagagtttaagcgagttgcaagaatctctacggtacagatgcaacttactatagccaaggtctattcgtattactatggcagcgaaaggagctttaaggttttaattaccccatagccatagattctactcggtctatctatcatgtaacactccgttgatgcgtactagaaaatgacaacgtaccgggcttgagggacatacagagacaattacagtaatcaagagtgtacccaactttaacgaactcagtaaaaaataaggaatgtcgacatcttaattttttatataaagcggtttggtattgattgtttgaagaattttcgggttggtgtttctttctgatgctacatagaagaacatcaaacaactaaaaaaatagtataat

SEQ ID NO: 22 - DNA, 785 bp, Artificial Sequence
  source: /note=“Description of Artificial Sequence Synthetic polynucleotide”
attatactatttttttagttgtttgatgttcttctatgtagcatcagaaagaaacaccaacccgaaaattcttcaaacaatcaataccaaaccgctttatataaaaaattaagatgtcgacattccttattttttactgagttcgttaaagttgggtacactcttgattactgtaattgtctctgtatgtccctcaagcccggtacgttgtcattttctagtacgcatcaacggagtgttacatgatagatagaccgagtagaatctatggctatggggtaattaaaaccttaaagctcctttcgctgccatagtaatacgaatagaccttggctatagtaagttgcatctgtaccgtagagattcttgcaactcgcttaaactctcgcttttagataatatttctccttattgcgcgcttcgttgaaaatttcgctaaacacggggtttaagttaaagtttacaggatttatccggaaattttcgcggaccccacacaattaagaattggctcgaagagtgataacgcatacttttcttttctttttttagttcctagcgtacctaacgtaggtaacatgatttggatcgtgggatgatacaaacaacgtaagatgaatagttccttcctcaattcttcttgcagcatcattttcttgaggcgctctgggcaaggtataaaaagttccattaatacgtctctaaaaaattaaatcatccatctcttaagcagtttttttgataatctcaaatgtacatcagtcaagcgtaactaaattacataa

SEQ ID NO: 23 - DNA, 875 bp, Artificial Sequence
  source: /note=“Description of Artificial Sequence Synthetic polynucleotide”
ttatgtattttagttacgcttgactgatgtacatttgagattatcaaaaaaactgcttaagagatagatggtttaattttttagagacgtattaatggaactttttataccttgcccagagcgcctcaagaaaatgatgctgaaagaagaattgaggaaggaactactcatcttacgttgtttgtatcatcccacgatccaaatcatgttacctacgttaggtacgctaggaactgaaaaaagaaaagaaaagtatgcgttatcactcttcgagccaattcttaattgtgtggggtccgcgaaaacttccggataaatcctgtaaacttaaacttaaaccccgtgtttagcgaaattttcaacgaagcgcgcaataaggagaaatattatataaaagcgagagtttaagcgaggttgcaagaatctctacggtacagatgcaacttactatagccaaggtctattcgtattggtatccaagcagtgaagctactcaggggaaaacatattttcagagatcaaagttatgtcagtctctttttcatgtgtaacttaacgtttgtgcaggtatcataccggcctccacataatttttgtggggaagacgttgttgtagcagtctccttatactctccaacaggtgtttaaagacttcttcaggcctcatagtctacatctggagacaacattagatagaagtttccacagaggcagctttcaatatactttcggctgtgtacatttcatcctgagtgagcgcatattgcataagtactcagtatataaagagacacaatatactccatacttgttgtgagtggttttagcgtattcagtataacaataagaattacatccaagactattaattaact

SEQ ID NO: 24 - DNA, 875 bp, Artificial Sequence
  source: /note=“Description of Artificial Sequence Synthetic polynucleotide”
agttaattaatagtcttggatgtaattcttattgttatactgaatacgctaaaaccactcacaacaagtatggagtatattgtgtctctttatatactgagtacttatgcaatatgcgctcactcaggatgaaatgtacacagccgaaagtatattgaaagctgcctctgtggaaacttctatctaatgttgtctccagatgtagactatgaggcctgaagaagtctttaaacacctgttggagagtataaggagactgctacaacaacgtcttccccacaaaaattatgtggaggccggtatgatacctgcacaaacgttaagttacacatgaaaaagagactgacataactttgatctctgaaaatatgttttcccctgagtagcttcactgcttggataccaatacgaatagaccttggctatagtaagttgcatctgtaccgtagagattcttgcaacctcgcttaaactctcgcttttatataatatttctccttattgcgcgcttcgttgaaaatttcgctaaacacggggtttaagtttaagtttacaggatttatccggaagttttcgcggaccccacacaattaagaattggctcgaagagtgataacgcatacttttcttttcttttttcagttcctagcgtacctaacgtaggtaacatgatttggatcgtgggatgatacaaacaacgtaagatgagtagttccttcctcaattcttctttcagcatcattttcttgaggcgctctgggcaaggtataaaaagttccattaatacgtctctaaaaaattaaaccatctatctcttaagcagtttttttgataatctcaaatgtacatcagtcaagcgtaactaaaatacataa

SEQ ID NO: 25 - DNA, 504 bp, Artificial Sequence
  source: /note=“Description of Artificial Sequence Synthetic polynucleotide”
agtacaggaacgattgtcttgataatatgtgaaaagtgcacacgaaattagagggtgtcctttacaagtattcttagaaacacattcaagagcacaaaagtcgatgctttaagggtcaaggtggtggaaaacttgactggaattcttgacgaaaaaacaagaaaaacgtgattcgagcaatcataaacatacagccccgttccaaccggatcttgaggtttcccattttagatggaaataagcagagcaaaataaaaatcttgaacaagtaatagtggtgactgcaggttacgttggcatataaagtccgggtgacctgggtttcctgcaccaccagcccccatatgctagcacaatgggttttctttatccccggtcataattactcattttgctatattcttcataacttaagtacgcagatagagaaaattaataatctcgatatatattaaagtaaatgaaaagtagaaaatttagccagaactcttttttgcttcgagt

SEQ ID NO: 26 - DNA, 493 bp, Artificial Sequence
  source: /note=“Description of Artificial Sequence Synthetic polynucleotide”
gtagcttcactgcttggataccaatacgaatagaccttggctatagtaagttgcatctgtaccgtagagattcttgcaacctcgcttaaactctcgcttttatataatatttctccttattgcgcgcttcgttgaaaatttcgctaaacacggggtttaagtttaagtttacaggatttatccggaagttttcgcggaccccacacaattaagaattggctcgaagagtgataacgcatacttttcttttcttttttcagttcctagcgtacctaacgtaggtaacatgatttggatcgtgggatgatacaaacaacgtaagatgagtagttccttcctcaattcttctttcagcatcattttcttgaggcgctctgggcaaggtataaaaagttccattaatacgtctctaaaaaattaaaccatctatctcttaagcagtttttttgataatctcaaatgtacatcagtcaagcgtaactaaaatacataa

SEQ ID NO: 27 - DNA, 517 bp, Artificial Sequence
  source: /note=“Description of Artificial Sequence Synthetic polynucleotide”
agctcagttgtcaagatttagtcattaagaagggccgcagcagctttttgtataatagagcgtcttttttgtttgtgaaaaaaattttatggtgagatattgttcgattctacgaagtcattttactagtttatggactctgatataagacagagttgacaaggaaatggtgccgtgattgtttccgtgtacagcttttgagaacttccttgaaaaccaatcatctagcactttcatttctggggaaaaacctggaaccaaatcttgaaaaataaattccccagaagttttccttattccgtgttctaatcttctcgttcactttgcagtgacattccacggccatgcgcaatttaccccgcccccggattttattgtccgtaccgccatttttcaatagattaaaaaggaacaaaaaatcatttcagaaggtttctttctcgggaaaacactagagtgtaaatattgaatatcaaacatcgaacgagagcatcttgaagatatttatgttctaaat

SEQ ID NO: 28 - DNA, 500 bp, Artificial Sequence
  source: /note=“Description of Artificial Sequence Synthetic polynucleotide”
gtgtttattcgcgatatgagttgtgatatcagagacagagagagtttatgtgcgtaacaggaacggagaaaaccagagtaattgagtattataagcaataaatcataaaaagacattctttctcgtgcaattttttggtattcgggataatcttctacttgaaacttctttttttcggtgtttaatttgcctattggtaaatatttttgccgccgaggttctcagtgattatattcgtattaagcgataaccgagacatgcatggagcggcggggctgatattttgtggggtacgaaagcatgattggtcagtgacactcaaaaaaagaaaacagccgtaaaatagtagattttgttaaactcccctttaaacctgtgatattgtaaaaagacgaagaatttaataatttaataattcattacggtatttatttcttcataaacagttacaacaccctaaagagaatttacaagttgagtaaaagacaagacacaaaatt

SEQ ID NO: 29 - DNA, 501 bp, Artificial Sequence
  source: /note=“Description of Artificial Sequence Synthetic polynucleotide”
cgcagcttcacttttaagtttctttttctcctcacggcgcaaccgctaacttaagctaatccttatgaatccggagaaaagcggggtcttttaactcaataaaattttccgaaatcctttttcctacgcgttttcttcgggaactagataggtggctcttccacctgtttttccatcattttagtttttcgcaagccatgcgtgccttttcgtttttgcgatggcgaagcagggctggaaaaattaacggtacgccgcctaacgatagtaataggccacgcaactggcgtggacgacaacaataagtcgcccattttttatgttttcaaaacctagcaacccccaccaaacttgtcatcgttcccggattcacaaatgatataaaaagcgattacaattctacattctaaccagatttgagatttcctctttctcaattcctcttatattagattataagaacaacaaattaaattacaaaaagacttataaagcaacata

SEQ ID NO: 30 - DNA, 531 bp, Artificial Sequence
  source: /note=“Description of Artificial Sequence Synthetic polynucleotide”
cgtctctttctgtggagaagaagatatttccccgagcagttttttttccatggggccccatattcccccgcctgcaggaaaacttggggaaagaggaaaaacacttcggataaaaacggtcaagaagctcttcgacgatttagtgccaccttcatgaaaaattccagagttttttccagctgctttgattttacagtccattattcggcgtctaacgattctgattaagaaacaacggaggaaaactcaaattctaatataatatttttaagtttatgaaggtggggtggtaagaaaagcaactaaaataatctacaagtcaattagtggtgaaaagcttcaacactggggaatgaataatatgtcatctagaaaaaattttatataaatactcagtgttttattcattattctcgattcattcacttcaattcctcttcatgagtaatagaaaccatcaagaaaagatatattcaaagcctcttatcaaggtttggttttgaaacacttttacaataaaatctgccaaaa

SEQ ID NO: 31 - DNA, 506 bp, Artificial Sequence
  source: /note=“Description of Artificial Sequence Synthetic polynucleotide”
tccgaaattattccctagaacaagcgggaaaaaggtccggggaaatggagtccgtgcgagttttgttaggatgactgccccacacatttcctcatcttataattttgtggaaaaattcatcgtgagagaaaatacgagtccatttctccagtgaaactaccgtagacatggaatatctgccattctaccccttattcaagtgcctttttttttttttttcatcccacattttattgctgcctcaatctccattaagaaaaaaaatttatataaccaaatgacatttttcctttcttctcaaactttgtaatgcgcctgtaactgcttctttttttattaaaaaacagcatggagttttttaataacttaaggaaacatacaaaaagatttgttcatttcactccaagtattttttaacgtatattgaaagttctcaatagcgaaaccacaagcagcaatacaaagagaattttattcgaacgcatagagtacacacactcaaagga

SEQ ID NO: 32 - DNA, 521 bp, Artificial Sequence
  source: /note=“Description of Artificial Sequence Synthetic polynucleotide”
cattatgagggcttccattattccccgcatttttattactctgaacaggaataaaaagaaaaaacccagtttaggaaattatccgggggcgaagaaatacgcgtagcgttaatcgaccccacgtccagggtttttccatggaggtttctggaaaaactgacgaggaatgtgattataaatccctttatgtgatgtctaagacttttaaggtacgcccgatgtttgcctattaccatcatagagacgtttcttttcgaggaatgcttaaacgactttgtttgacaaaaatgttgcctaagggctctatagtaaaccatttggaagaaagatttgacgactttttttttttggatttcgatcctataatccttcctcctgaaaagaaacatataaatagatatgtattattcttcaaaacattctcttgttcttgtgctttttttttaccatatatcttacttttttttttctctcagagaaacaagcaaaacaaaaagcttttcttttcactaacgtatatg